(19) United States
(12) Patent Application Publication
(10) Pub. No.: US 2025/0206820 A1
(43) Pub. Date: Jun. 26, 2025

(54) ILT3 AND CD3 BINDING AGENTS AND METHODS OF USE THEREOF

(71) Applicant: NGM Biopharmaceuticals, Inc., South San Francisco, CA (US)

(72) Inventors: Keith AKAMA, San Francisco, CA (US); Rujin CHENG, Burlingame, CA (US); Anjushree R. IYER, San Mateo, CA (US); Vicky Yi-Bing LIN, Cupertino, CA (US); Lee B. RIVERA, Daly City, CA (US); Julie M. RODA, Pacifica, CA (US); Jie TANG, Palo Alto, CA (US); Hong YANG, San Mateo, CA (US)

(21) Appl. No.: 18/852,326

(22) PCT Filed: Mar. 28, 2023
(86) PCT No.: PCT/US2023/065027

§ 371 Date: Sep. 27, 2024

Related U.S. Application Data
(60) Provisional application No. 63/325,101, filed on Mar. 29, 2022.
(60) Provisional application No. 63/386,634, filed on Dec. 8, 2022.

(51) Int. Cl. C07K 16/28; A61K 39/00; A61P 35/00

(52) U.S. Cl. CPC C07K 16/2809; A61P 35/00; C07K 16/2803; A61K 2039/505; C07K 2317/24; C07K 2317/31; C07K 2317/522; C07K 2317/524; C07K 2317/526; C07K 2317/55; C07K 2317/565; C07K 2317/622

(57) ABSTRACT

The present disclosure relates to ILT3×CD3 binding agents, compositions comprising thereof, and methods of use thereof. The present disclosure also relates to polynucleotides and vectors encoding such ILT3×CD 3 binding agents.

### CROSS-REFERENCE TO RELATED APPLICATIONS

This application claims priority to U.S. Provisional Application No. 63/325,101 filed Mar. 29, 2022 and U.S. Provisional Application No. 63/386,634 filed Dec. 8, 2022, the content of each of which is incorporated by reference in its entirety herein.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “13370-172-228_SEQLISTING.xml”, was created on Mar. 24, 2023, and is 189,173 bytes in size.

### 1. FIELD

The present disclosure relates to ILT3×CD3 binding agents that bind immunoglobulin-like transcript 3 (ILT3) and CD3, compositions comprising thereof, and methods of use thereof. The present disclosure also relates to polynucleotides and vectors encoding such ILT3×CD3 binding agents.

### 2. BACKGROUND

The basis for immunotherapy is the manipulation and/or modulation of the immune system, including both innate immune responses and adaptive immune responses. The general aim of immunotherapy is to treat diseases by controlling the immune response to a “foreign agent,” for example a pathogen or a tumor cell. However, in some instances, immunotherapy is used to treat autoimmune diseases, which may arise from an abnormal immune response against proteins, molecules, and/or tissues normally present in the body. Immunotherapy may include methods to induce or enhance specific immune responses or to inhibit or reduce specific immune responses.

The concept of cancer immunosurveillance is based on the theory that the immune system can recognize tumor cells, mount an immune response, and suppress the development and/or growth of a tumor. However, it is clear that many cancerous cells have developed mechanisms and/or hijacked normal inhibitory mechanisms to evade the immune system, which can allow for uninhibited growth of tumors. Cancer/tumor immunotherapy (immuno-oncology) focuses on the development of new and novel agents that can activate and/or boost the immune system to achieve a more effective attack against cancer/tumor cells resulting in increased killing of cancer/tumor cells and/or inhibition of cancer/tumor growth. There remains a need in the art for more effective molecules for treating various diseases or disorders.

### 3. SUMMARY

In one aspect, the present disclosure provides a binding agent comprising a first binding region that binds to human ILT3 and a second binding region that binds to human CD3, wherein the CD3 binding region comprises an anti-CD3 scFv.

In certain embodiments, the first binding region comprises an anti-ILT3 Fab. In certain embodiments, the binding affinity of the first binding region for human ILT3 is higher than the binding affinity of the second binding region for human CD3. In certain embodiments, the binding affinity of the first binding region for human ILT3 is between about 10 folds and about 100 folds higher than the binding affinity of the second binding region for human CD3. In certain embodiments, the binding agent further comprises a Fc region.

In certain embodiments, the binding agent comprises (i) a first polypeptide comprising the anti-CD3 scFv, a first CH2 domain, and a first CH3 domain; (ii) a second polypeptide comprising a VH domain of the first binding region, a CH1 domain, a second CH2 domain, and a second CH3 domain; and (iii) a third polypeptide comprising a VL domain of the first binding region and a CL domain, wherein the VH domain of the first binding region, the CH1 domain, the VL domain of the first binding region, and the CL domain form the anti-ILT3 Fab, and the first CH2 domain, the second CH2 domain, the first CH3 domain, and the second CH3 domain form the Fc region.

In certain embodiments, the first polypeptide comprises one or more amino acid mutations that form an engineered cavity, and the second polypeptide comprising one or more amino acid mutations that form an engineered protuberance, and wherein the first polypeptide dimerizes with the second polypeptide via positioning of the protuberance into the cavity.

In certain embodiments, the second binding region comprises a VH domain comprising a HCDR1, a HCDR2, and a HCDR3 of the amino acid sequence set forth in SEQ ID NO: 149; and a VL domain comprising a LCDR1, a LCDR2, and a LCDR3 of the amino acid sequence set forth in SEQ ID NO:150.

In certain embodiments, in the second binding region, the VH domain of the second binding region comprises the HCDR1 comprising the amino acid sequence of SEQ ID NO:152, the HCDR2 comprising the amino acid sequence of SEQ ID NO: 153, and the HCDR3 comprising the amino acid sequence of SEQ ID NO:154; and the VL domain of the second binding region comprises the LCDR1 comprising the amino acid sequence of SEQ ID NO: 155, the LCDR2 comprising the amino acid sequence of SEQ ID NO: 156, and the LCDR3 comprising the amino acid sequence of SEQ ID NO: 157.

In certain embodiments, the first binding region comprises a VH domain comprising a HCDR1, a HCDR2, and a HCDR3 of the amino acid sequence set forth in SEQ ID NO:17, and a VL domain comprising a LCDR1, a LCDR2, and a LCDR3 of the amino acid sequence set forth in SEQ ID NO:18.

In certain embodiments, in the first binding region, (a) the VH domain of the first binding region comprises the HCDR1 comprising the amino acid sequence of SEQ ID NO:1, the HCDR2 comprising the amino acid sequence of SEQ ID NO:2, and the HCDR3 comprising the amino acid sequence of SEQ ID NO:3; and the VL domain of the first binding region comprises the LCDR1 comprising the amino acid sequence of SEQ ID NO:4, the LCDR2 comprising the amino acid sequence of SEQ ID NO:5, and the LCDR3 comprising the amino acid sequence of SEQ ID NO: 6; (b) the VH domain of the first binding region comprises the HCDR1 comprising the amino acid sequence of SEQ ID NO:7, the HCDR2 comprising the amino acid sequence of SEQ ID NO:8, and the HCDR3 comprising the amino acid sequence of SEQ ID NO:3; and the VL domain of the first binding region comprises the LCDR1 comprising the amino acid sequence of SEQ ID NO:4, the LCDR2 comprising the amino acid sequence of SEQ ID NO:5, and the LCDR3 comprising the amino acid sequence of SEQ ID NO:6; (c) the VH domain of the first binding region comprises the HCDR1 comprising the amino acid sequence of SEQ ID NO:1, the HCDR2 comprising the amino acid sequence of SEQ ID NO:9, and the HCDR3 comprising the amino acid sequence of SEQ ID NO: 3; and the VL domain of the first binding region comprises the LCDR1 comprising the amino acid sequence of SEQ ID NO:4, the LCDR2 comprising the amino acid sequence of SEQ ID NO:5, and the LCDR3 comprising the amino acid sequence of SEQ ID NO:6; (d) the VH domain of the first binding region comprises the HCDR1 comprising the amino acid sequence of SEQ ID NO: 10, the HCDR2 comprising the amino acid sequence of SEQ ID NO:2, and the HCDR3 comprising the amino acid sequence of SEQ ID NO:3; and the VL domain of the first binding region comprises the LCDR1 comprising the amino acid sequence of SEQ ID NO:4, the LCDR2 comprising the amino acid sequence of SEQ ID NO:5, and the LCDR3 comprising the amino acid sequence of SEQ ID NO: 6; or (e) the VH domain of the first binding region comprises the HCDR1 comprising the amino acid sequence of SEQ ID NO:11, the HCDR2 comprising the amino acid sequence of SEQ ID NO: 12, and the HCDR3 comprising the amino acid sequence of SEQ ID NO: 13; and the VL domain of the first binding region comprises the LCDR1 comprising the amino acid sequence of SEQ ID NO:14, the LCDR2 comprising the amino acid sequence of SEQ ID NO:15, and the LCDR3 comprising the amino acid sequence of SEQ ID NO: 16.

In certain embodiments, (i) the first binding region comprises the VH domain having at least 90% sequence identity to the amino acid sequence of SEQ ID NO:17, and the VL domain having at least 90% sequence identity to the amino acid sequence of SEQ ID NO:18; and the second binding region comprises the VH domain having at least 95% sequence identity to the amino acid sequence of SEQ ID NO:149, and the VL domain having at least 90% sequence identity to the amino acid sequence of SEQ ID NO:150; or (ii) the first binding region comprises the VH domain comprising the amino acid sequence of SEQ ID NO: 17, and the VL domain comprising the amino acid sequence of SEQ ID NO: 18; and the second binding region comprises the VH domain comprising the amino acid sequence of SEQ ID NO: 149, and the VL domain comprising the amino acid sequence of SEQ ID NO:150.

In another aspect, the present disclosure provides a binding agent comprising a first binding region that binds to human ILT3 and a second binding region that binds to human CD3, wherein the second binding region comprises a VH domain comprising a HCDR1, a HCDR2, and a HCDR3 of the amino acid sequence set forth in SEQ ID NO: 149, and a VL domain comprising a LCDR1, a LCDR2, and a LCDR3 of the amino acid sequence set forth in SEQ ID NO:150.

In certain embodiments, the VH domain of the second binding region comprises the HCDR1 comprising the amino acid sequence of SEQ ID NO: 152, the HCDR2 comprising the amino acid sequence of SEQ ID NO:153, and the HCDR3 comprising the amino acid sequence of SEQ ID NO:154; and the VL domain of the second binding region comprises the LCDR1 comprising the amino acid sequence of SEQ ID NO: 155; the LCDR2 comprising the amino acid sequence of SEQ ID NO: 156; and the LCDR3 comprising the amino acid sequence of SEQ ID NO: 157.

In certain embodiments, the first binding region comprises a VH domain comprising a HCDR1, a HCDR2, and a HCDR3 of the amino acid sequence set forth in SEQ ID NO:17, and a VL domain comprising a LCDR1, a LCDR2, and a LCDR3 of the amino acid sequence set forth in SEQ ID NO:18

In certain embodiments, in the first binding region, (a) the VH domain of the first binding region comprises the HCDR1 comprising the amino acid sequence of SEQ ID NO:1, the HCDR2 comprising the amino acid sequence of SEQ ID NO:2, and the HCDR3 comprising the amino acid sequence of SEQ ID NO:3; and the VL domain of the first binding region comprises the LCDR1 comprising the amino acid sequence of SEQ ID NO:4, the LCDR2 comprising the amino acid sequence of SEQ ID NO:5, and the LCDR3 comprising the amino acid sequence of SEQ ID NO: 6; (b) the VH domain of the first binding region comprises the HCDR1 comprising the amino acid sequence of SEQ ID NO:7, the HCDR2 comprising the amino acid sequence of SEQ ID NO:8, and the HCDR3 comprising the amino acid sequence of SEQ ID NO:3; and the VL domain of the first binding region comprises the LCDR1 comprising the amino acid sequence of SEQ ID NO:4, the LCDR2 comprising the amino acid sequence of SEQ ID NO:5, and the LCDR3 comprising the amino acid sequence of SEQ ID NO:6; (c) the VH domain of the first binding region comprises the HCDR1 comprising the amino acid sequence of SEQ ID NO:1, the HCDR2 comprising the amino acid sequence of SEQ ID NO:9, and the HCDR3 comprising the amino acid sequence of SEQ ID NO: 3; and the VL domain of the first binding region comprises the LCDR1 comprising the amino acid sequence of SEQ ID NO:4, the LCDR2 comprising the amino acid sequence of SEQ ID NO:5, and the LCDR3 comprising the amino acid sequence of SEQ ID NO:6; (d) the VH domain of the first binding region comprises the HCDR1 comprising the amino acid sequence of SEQ ID NO:10, the HCDR2 comprising the amino acid sequence of SEQ ID NO:2, and the HCDR3 comprising the amino acid sequence of SEQ ID NO:3; and the VL domain of the first binding region comprises the LCDR1 comprising the amino acid sequence of SEQ ID NO:4, the LCDR2 comprising the amino acid sequence of SEQ ID NO:5, and the LCDR3 comprising the amino acid sequence of SEQ ID NO: 6; or (e) the VH domain of the first binding region comprises the HCDR1 comprising the amino acid sequence of SEQ ID NO: 11, the HCDR2 comprising the amino acid sequence of SEQ ID NO: 12, and the HCDR3 comprising the amino acid sequence of SEQ ID NO: 13; and the VL domain of the first binding region comprises the LCDR1 comprising the amino acid sequence of SEQ ID NO:14, the LCDR2 comprising the amino acid sequence of SEQ ID NO: 15, and the LCDR3 comprising the amino acid sequence of SEQ ID NO: 16.

In certain embodiments, (i) the first binding region comprises the VH domain having at least 90% sequence identity to the amino acid sequence of SEQ ID NO:17, and the VL domain having at least 90% sequence identity to the amino acid sequence of SEQ ID NO:18; and the second binding region comprises the VH domain having at least 95% sequence identity to the amino acid sequence of SEQ ID NO:149, and the VL domain having at least 90% sequence identity to the amino acid sequence of SEQ ID NO:150; or (ii) the first binding region comprises the VH domain comprising the amino acid sequence of SEQ ID NO: 17, and the VL domain comprising the amino acid sequence of SEQ ID NO: 18; and the second binding region comprises the VH domain comprising the amino acid sequence of SEQ ID NO: 149, and the VL domain comprising the amino acid sequence of SEQ ID NO:150.

In certain embodiments, the first binding region comprises an anti-ILT3 Fab. In certain embodiments, the second binding region comprises an anti-CD3 scFv. In certain embodiments, the binding agent further comprises a Fc region.

In certain embodiments, the binding agent comprises: (i) a first polypeptide comprising the anti-CD3 scFv, a first CH2 domain, and a first CH3 domain; (ii) a second polypeptide comprising the VH domain of the first binding region, a CH1 domain, a second CH2 domain and a second CH3 domain; and (iii) a third polypeptide comprising the VL domain of the first binding region and a CL domain, wherein the VH domain of the first binding region, the CH1 domain, the VL domain of the first binding region, and the CL domain form the anti-ILT3 Fab, and the first CH2 domain, the second CH2 domain, the first CH3 domain, and the second CH3 domain form the Fc region.

In certain embodiments, the first polypeptide comprising one or more amino acid mutations that form an engineered cavity, and the second polypeptide comprising one or more amino acid mutations that form an engineered protuberance, and wherein the first polypeptide dimerizes with the second polypeptide via positioning of the protuberance into the cavity.

In certain embodiments, (i) the first polypeptide comprises the amino acid sequence of SEQ ID NO:147, the second polypeptide comprises the amino acid sequence of SEQ ID NO:19, and the third polypeptide comprises the amino acid sequence of SEQ ID NO:20, or (ii) the first polypeptide comprises an amino acid sequence having at least 90% sequence identity of the amino acid sequence of SEQ ID NO: 147, the second polypeptide comprises an amino acid sequence having at least 90% sequence identity of the amino acid sequence of SEQ ID NO: 19, and the third polypeptide comprises an amino acid sequence having at least 90% sequence identity of the amino acid sequence of SEQ ID NO:20.

In certain embodiments, the first binding region comprises two identical anti-ILT3 Fabs, and the second binding region comprises an anti-CD3 scFv.

In certain embodiments, the binding agent comprises: (i) a first polypeptide comprising the ant-CD3 scFv, a first CH2 domain, and a first CH3 domain; (ii) a second polypeptide comprising a first VH domain, a second VH domain, a first CH1 domain, a second CH1 domain, a second CH2 domain, and a second CH3 domain, wherein each of the first and second VH domains comprises the VH domain of the first binding region; (iii) a third polypeptide comprising a first VL domain and a first CL domain, wherein the first VL domain comprises the VL domain of the first binding region; and (iv) a fourth polypeptide comprising a second VL domain and a second CL domain, wherein the second VL domain comprises the VL domain of the first binding region, wherein the first VH domain and the first CH1 domain of the second polypeptide and the first VL domain and the first CL domain of the third polypeptide form a first Fab region, the second VH domain and the second CH1 domain of the second polypeptide and the second VL domain and the second CL domain of the fourth polypeptide form a second Fab region, and the first CH2 domain, the second CH2 domain, the first CH3 domain, and the second CH3 domain form the Fc region.

In certain embodiments, the first polypeptide comprising one or more amino acid mutations that form an engineered cavity, and the second polypeptide comprising one or more amino acid mutations that form an engineered protuberance, and wherein the first polypeptide dimerizes with the second polypeptide via positioning of the protuberance into the cavity.

In certain embodiments, (i) the first polypeptide comprises the amino acid sequence of SEQ ID NO:147, the second polypeptide comprises the amino acid sequence of SEQ ID NO:169, the third polypeptide comprises the amino acid sequence of SEQ ID NO:20, and the fourth polypeptide comprises the amino acid sequence of SEQ ID NO:20; or (ii) the first polypeptide comprises an amino acid sequence having at least 90% sequence identity of the amino acid sequence of SEQ ID NO:147, the second polypeptide comprises an amino acid sequence having at least 90% sequence identity of the amino acid sequence of SEQ ID NO: 169, the third polypeptide comprises an amino acid sequence having at least 90% sequence identity of the amino acid sequence of SEQ ID NO:20, and the fourth polypeptide comprises an amino acid sequence having at least 90% sequence identity of the amino acid sequence of SEQ ID NO:20.

In certain embodiments, the anti-CD3 scFv comprises the amino acid sequence of SEQ ID NO: 151. In certain embodiments, the binding agent is a humanized antibody.

In another aspect, the present disclosure provides a binding agent comprises: (i) a first polypeptide comprising an scFv that binds to human CD3, a first CH2 domain, and a first CH3 domain; (ii) a second polypeptide comprising a VH domain that binds to human ILT3, a CH1 domain, a second CH2 domain and a second CH3 domain; and (iii) a third polypeptide comprising a VL domain that binds to human ILT3, and a CL domain, wherein the scFv that binds to human CD3 comprises a VH domain comprising a HCDR1, a HCDR2, and a HCDR3 of the amino acid sequence set forth in SEQ ID NO: 149, and a VL domain comprising a LCDR1, a LCDR2, and a LCDR3 of the amino acid sequence set forth in SEQ ID NO:150; and wherein the VH domain that binds to human ILT3 comprises a HCDR1, a HCDR2, and a HCDR3 of the amino acid sequence set forth in SEQ ID NO:17, and the VL domain that binds to human ILT3 comprises a LCDR1, a LCDR2, and a LCDR3 of the amino acid sequence set forth in SEQ ID NO:18.

In certain embodiments, (a) the HCDR1 of the scFv comprises the amino acid sequence of SEQ ID NO: 152, the HCDR2 of the scFv comprises the amino acid sequence of SEQ ID NO: 153, the HCDR3 of the scFv comprises the amino acid sequence of SEQ ID NO: 154, the LCDR1 of the scFv comprises the amino acid sequence of SEQ ID NO: 155, the LCDR2 of the scFv comprises the amino acid sequence of SEQ ID NO:156, and the LCDR3 of the scFv comprises the amino acid sequence of SEQ ID NO:157; and (b) in the VH domain that binds to human ILT3 and the VL domain that binds to human ILT3 (i) the HCDR1 comprises the amino acid sequence of SEQ ID NO:1; the HCDR2 comprises the amino acid sequence of SEQ ID NO:2; the HCDR3 comprises the amino acid sequence of SEQ ID NO:3; the LCDR1 comprises the amino acid sequence of SEQ ID NO:4; the LCDR2 comprises the amino acid sequence of SEQ ID NO:5; and the LCDR3 comprises the amino acid sequence of SEQ ID NO:6; (ii) the HCDR1 comprises the amino acid sequence of SEQ ID NO:7; the HCDR2 comprises the amino acid sequence of SEQ ID NO: 8; the HCDR3 comprises the amino acid sequence of SEQ ID NO:3; the LCDR1 comprises the amino acid sequence of SEQ ID NO:4; the LCDR2 comprises the amino acid sequence of SEQ ID NO: 5; and the LCDR3 comprises the amino acid sequence of SEQ ID NO:6; (iii) the HCDR1 comprises the amino acid sequence of SEQ ID NO:1; the HCDR2 comprises the amino acid sequence of SEQ ID NO:9; the HCDR3 comprises the amino acid sequence of SEQ ID NO:3; the LCDR1 comprises the amino acid sequence of SEQ ID NO:4; the LCDR2 comprises the amino acid sequence of SEQ ID NO:5; and the LCDR3 comprises the amino acid sequence of SEQ ID NO:6; (iv) the HCDR1 comprises the amino acid sequence of SEQ ID NO:10; the HCDR2 comprises the amino acid sequence of SEQ ID NO:2; the HCDR3 comprises the amino acid sequence of SEQ ID NO: 3; the LCDR1 comprises the amino acid sequence of SEQ ID NO:4; the LCDR2 comprises the amino acid sequence of SEQ ID NO:5; and the LCDR3 comprises the amino acid sequence of SEQ ID NO: 6; or (v) the HCDR1 comprises the amino acid sequence of SEQ ID NO:11; the HCDR2 comprises the amino acid sequence of SEQ ID NO:12; the HCDR3 comprises the amino acid sequence of SEQ ID NO:13; the LCDR1 comprises the amino acid sequence of SEQ ID NO:14; the LCDR2 comprises the amino acid sequence of SEQ ID NO:15; and the LCDR3 comprises the amino acid sequence of SEQ ID NO:16.

In certain embodiments, the VH domain of the scFv that binds to human CD3 comprises the amino acid sequence of SEQ ID NO:149, and the VL domain of the scFv that binds to human CD3 comprises the amino acid sequence of SEQ ID NO:150; and the VH domain that binds to human ILT3 comprises the amino acid sequence of SEQ ID NO:17, and the VL domain that binds to human ILT3 comprises the amino acid sequence of SEQ ID NO:18. In certain embodiments, the scFv comprises the amino acid sequence of SEQ ID NO:151.

In another aspect, the present disclosure provides an isolated polynucleotide encoding the binding agent disclosed herein.

In another aspect, the present disclosure provides a vector comprising the polynucleotide disclosed herein.

In another aspect, the present disclosure provides an isolated cell comprising the polynucleotide or the vector of disclosed herein.

In another aspect, the present disclosure provides an isolated cell producing the binding agent disclosed herein.

In another aspect, the present disclosure provides a pharmaceutical composition comprising the binding agent disclosed herein, the isolated polynucleotide disclosed herein, the vector disclosed herein, or the isolated cell disclosed herein, and a pharmaceutically acceptable excipient.

In another aspect, the present disclosure provides a method of directing a T cell to a cancer or tumor cell expressing ILT3, comprising contacting the T cell with an effective amount of the binding agent disclosed herein or the pharmaceutical composition disclosed herein.

In certain embodiments, the T cell induces the killing of the cancer cell or tumor cell expressing ILT3. In certain embodiments, the cancer or tumor cell is a hematological cancer or tumor cell. In certain embodiments, the hematological cancer or tumor cell is selected from the group consisting of an acute myeloid leukemia (AML) cell, a M4/M5 AML cell, a chronic myelomonocytic leukemia (CMML) cell, a B-cell acute lymphoblastic leukemia (B-ALL) cell, a chronic lymphocytic leukemia (CLL) cell, a diffuse large B-cell lymphoma (DLBCL) cell, a mantle cell lymphoma (MCL) cell, a multiple myeloma (MM) cell, a myelodysplastic syndrome (MDS) cell, a Hodgkin lymphoma cell, a lymphoplasmacytic lymphoma (LPL) cell, a follicular lymphoma cell, a Burkitt lymphoma cell, a blastic plasmacytoid dendritic cell neoplasm (BPDCN) cell, a marginal zone lymphoma cell, or a mucosa-associated lymphoid tissue (MALT) lymphoma cell. In certain embodiments, the T cell fails to induce killing of a normal hematopoietic stem cell (HSC).

In another aspect, the present disclosure provides a method of activating a T cell, comprising contacting the T cell with an effective amount of the binding agent disclosed herein or the pharmaceutical composition disclosed herein, wherein the second binding region binds the T cell.

In certain embodiments, the T cell is a naïve T cell. In certain embodiments, the T cell is polyclonally expanded from a population of PBMCs.

In another aspect, the present disclosure provides a method of killing or inhibiting the proliferation of a cancer or tumor cell expressing ILT3, comprising contacting the cancer or tumor cell with the binding agent disclosed herein or the pharmaceutical composition disclosed herein.

In certain embodiments, the binding agent activates a T cell. In certain embodiments, the activated T cell induces the killing of the cancer or tumor cell.

In certain embodiments, the cancer or tumor cell comprises a hematological cancer or tumor cell. In certain embodiments, the hematological cancer or tumor cell is selected from the group consisting of an acute myeloid leukemia (AML) cell, a M4/M5 AML cell, a chronic myelomonocytic leukemia (CMML) cell, a B-cell acute lymphoblastic leukemia (B-ALL) cell, a chronic lymphocytic leukemia (CLL) cell, a diffuse large B-cell lymphoma (DLBCL) cell, a mantle cell lymphoma (MCL) cell, a multiple myeloma (MM) cell, a myelodysplastic syndrome (MDS) cell, a Hodgkin lymphoma cell, a lymphoplasmacytic lymphoma (LPL) cell, a follicular lymphoma cell, a Burkitt lymphoma cell, a blastic plasmacytoid dendritic cell neoplasm (BPDCN) cell, a marginal zone lymphoma cell, or a mucosa-associated lymphoid tissue (MALT) lymphoma cell.

In another aspect, the present disclosure provides a method of treating a cancer or a tumor expressing ILT3 in a subject, comprising administering an effective amount of the binding agent disclosed herein or the pharmaceutical composition disclosed herein to the subject.

In certain embodiments, the cancer or tumor comprises a hematological cancer or tumor. In certain embodiments, the hematological cancer or tumor is selected from the group consisting of acute myeloid leukemia (AML), a M4/M5 AML chronic myelomonocytic leukemia (CMML), B-cell acute lymphoblastic leukemia (B-ALL), chronic lymphocytic leukemia (CLL), diffuse large B-cell lymphoma (DLBCL), mantle cell lymphoma (MCL), multiple myeloma (MM), myelodysplastic syndrome (MDS), Hodgkin lymphoma, lymphoplasmacytic lymphoma (LPL), follicular lymphoma, Burkitt lymphoma, blastic plasmacytoid dendritic cell neoplasm (BPDCN), marginal zone lymphoma, or mucosa-associated lymphoid tissue (MALT) lymphoma.

### 4. BRIEF DESCRIPTION OF THE DRAWINGS

FIG. 1 shows an exemplary ILT3×CD3 bispecific antibody (ABX1446) disclosed herein. The left arm (second binding region of the antibody) represents an anti-CD3 scFv provided herein and the right arm (first binding region of the antibody) represents an anti-ILT3 Fab provided herein.

FIG. 2 shows the results of a T-cell dependent cellular cytotoxicity (TDCC) assay with different ILT3×CD3 bispecific antibodies (hz45G10-2B2, 16C5-2B2 and 12A12-2B2). Anti-KLH was used as a negative control.

FIG. 3 shows the results of a T-cell dependent cellular cytotoxicity (TDCC) assay with different ILT3×CD3 bispecific antibodies (hz45G10-1G4, 16C5-1G4 and 12A12-1G4). Anti-KLH was used as a negative control.

FIG. 4 shows the results of a TNFα cytokine production assay with different ILT3×CD3 bispecific antibodies (hz45G10-2B2, 3A3-2B2 and 12A12-2B2). Anti-KLH was used as a negative control.

FIG. 5 shows the results of a T-cell dependent cellular cytotoxicity (TDCC) assay in AML cells with ILT3×CD3 bispecific antibodies. Anti-KLH was used as a negative control.

FIG. 6 shows the results of a TNFα cytokine production assay with ILT3×CD3 bispecific antibodies. Anti-KLH was used as a negative control.

FIG. 7 shows various formats of the exemplary ILT3×CD3 bispecific antibody provided herein.

FIG. 8 shows T-cell dependent cellular cytotoxicity (TDCC) activity of various formats (FIG. 7) of ILT3×CD3 bispecific antibodies in MOLM13 cells, when expanded T cells were used as effectors. Anti-KLH represents the negative control and Vibecotamab (CD123×CD3 bispecific) represents the positive control.

FIG. 9 shows TNFα cytokine release when a whole blood sample was added to plates that were pre-coated with ILT3×CD3 bispecific antibody in various formats (FIG. 7) at the indicated concentrations (for each treatment group, the bars from left to right represent 50 μg/ml, 10 μg/ml, 1 μg/ml, and 0.1 μg/ml respectively).

FIG. 10 shows TNFα cytokine release when a whole blood sample was added to culture medium containing soluble ILT3×CD3 bispecific antibody in various formats (FIG. 7) at the indicated concentrations (for each treatment group, the bars from left to right represent 100 μg/ml, 10 μg/ml, 1 μg/ml, and 0.1 μg/ml respectively).

FIG. 11 shows that ILT3×CD3 bispecific antibody (ABX1446 and ABX1520) induced potent apoptosis of ILT3 positive (ILT3+) AML cells (MOLM13) when expanded T cells were used as effectors. Vibecotamab, a CD123×CD3 bispecific, was used as a positive control. Anti-KLH was used as a negative control.

FIG. 12 shows that ILT3×CD3 bispecific antibody (ABX1446 and ABX1520) induced potent apoptosis of ILT3 positive (ILT3+) AML cells (MOLM13) when naive T cells were used as effectors. Vibecotamab, a CD123×CD3 bispecific, was used as a positive control. Anti-KLH was used as a negative control.

FIG. 13 shows that ILT3×CD3 bispecific antibody (ABX1446 and ABX1520) induced apoptosis of OCI-AML-2 cells with low ILT3 expression. Vibecotamab, a CD123×CD3 bispecific, was used as a positive control. Anti-KLH was used as a negative control.

FIG. 14 shows that ILT3×CD3 bispecific antibody (ABX1446 and ABX1520) induced apoptosis of NALM-1 cells that have low ILT3 expression. Vibecotamab, a CD123×CD3 bispecific, was used as a positive control. Anti-KLH was used as a negative control.

FIG. 15 shows that ILT3×CD3 bispecific antibody (ABX1446 and ABX1520) induced apoptosis of OCI-AML-2 cells, which had low ILT3 expression, when naive T cells were used as effectors. Vibecotamab, a CD123×CD3 bispecific, was used as a positive control. Anti-KLH was used as a negative control.

FIG. 16 shows that ILT3×CD3 bispecific antibody (ABX1446 and ABX1520) induced apoptosis of NALM-1 cells, which had low ILT3 expression, when naive T cells were used as effectors. Vibecotamab, a CD123×CD3 bispecific, was used as a positive control. Anti-KLH was used as a negative control.

FIG. 17 shows that ILT3×CD3 bispecific antibody (ABX1446 and ABX1520) failed to induce apoptosis of ILT3 knock-out THP-1 cells. Vibecotamab, a CD123×CD3 bispecific, was used as a positive control. Anti-KLH was used as a negative control.

FIG. 18 shows the results of TNFα cytokine production in PBMCs incubated with increasing concentrations (for each treatment group, the bars from left to right represent 100 μg/ml, 10 μg/ml, 1 μg/ml, and 0.1 μg/ml respectively) of plate-coated format anti-KLH (negative control), Vibecotamab (CD123× CD3 bispecific) and the ILT3×CD3 bispecific antibody (ABX1446 and ABX1520). Soluble staphylococcal enterotoxin B (SEB) was included as a positive control.

FIG. 19 shows the results of TNFα cytokine production in PBMCs incubated with increasing concentrations (for each treatment group, the bars from left to right represent 10 μg/ml, 1 μg/ml, 0.1 μg/ml, and 0.01 μg/ml respectively) of soluble anti-KLH (negative control), SEB (staphylococcal enterotoxin B; positive control), Vibecotamab (CD123×CD3 bispecific) and the ILT3×CD3 bispecific antibody (ABX1446 and ABX1520) provided herein.

FIG. 20 shows the results of TNFα cytokine secretion assay in whole blood incubated with increasing concentrations (for each treatment group, the bars from left to right represent 10 μg/ml, 1 μg/ml, 0.1 μg/ml, and 0.01 μg/ml respectively) of plate-coated anti-KLH (negative control), Vibecotamab (CD123×CD3 bispecific), and ILT3×CD3 bispecific antibody in two different formats, including F0 (ABX1446) and F13 (ABX1520) (see FIG. 7). Soluble staphylococcal enterotoxin B (SEB) was included as a positive control.

FIG. 21 shows the results of TNFα cytokine secretion assay in whole blood incubated with increasing concentrations (for each treatment group, the bars from left to right represent 10 μg/ml, 1 μg/ml, 0.1 μg/ml, and 0.01 μg/ml respectively) of soluble anti-KLH (negative control), SEB (staphylococcal enterotoxin B; positive control), Vibecotamab (CD123×CD3 bispecific), and ILT3×CD3 bispecific antibody in two different formats, including F0 (ABX1446) and F14 (ABX1521) (see FIG. 7).

FIG. 22 shows that ILT3×CD3 bispecific antibody (ABX1446) induced low TNFα cytokine secretion in TDCC assays with ILT3 positive (ILT3+) AML cells (MOLM13) when naive T cells were used as effectors. Anti-KLH represents the negative control (negative control) and Vibecotamab (CD123×CD3 bispecific) represents the positive control.

FIG. 23 shows that ILT3×CD3 bispecific antibody (ABX1446) induced low IL6 cytokine secretion in TDCC assays with ILT3 positive (ILT3+) AML cells (MOLM13) when naive T cells were used as effectors. Anti-KLH represents the negative control and Vibecotamab (CD123×CD3 bispecific) represents the positive control.

FIG. 24 shows the results of a T-cell dependent cellular cytotoxicity (TDCC) assay with anti-KLH (negative control), Vibecotamab (CD123×CD3 bispecific), and ILT3×CD3 bispecific antibody ABX1446 and Flotetuzumab (CD123×CD3 DART).

FIG. 25 shows the results of a TNFα cytokine secretion assay in whole blood with increasing concentrations (for each treatment group, the bars from left to right represent 10 μg/ml, 1 μg/ml, 0.1 μg/ml, and 0.01 μg/ml respectively) of soluble anti-KLH (negative control), SEB (staphylococcal enterotoxin B; positive control), Vibecotamab (CD123×CD3 bispecific), ILT3×CD3 bispecific antibody ABX1446 and Flotetuzumab (CD123× CD3 DART).

FIG. 26 shows expansion of CD3 positive (CD3+) T cells (as a percentage of total PBMCs) in PBMCs isolated from an M5 AML patient at increasing concentrations (for each treatment group, the bars from left to right represent 0.1 μg/ml, 1 μg/ml, and 10 μg/ml respectively) of anti-KLH (negative control), Vibecotamab (CD123×CD3 bispecific), and ILT3×CD3 bispecific antibody ABX1446.

FIG. 27 shows expansion of CD25 positive (CD25+) T cells (as a percentage of total T cells) in PBMCs isolated from an M5 AML patient with increasing concentrations (for each treatment group, the bars from left to right represent 0.1 μg/ml, 1 μg/ml, and 10 μg/ml respectively) of anti-KLH (negative control), Vibecotamab (CD123×CD3 bispecific), and ILT3×CD3 bispecific antibody ABX1446.

FIG. 28 shows that ILT3×CD3 bispecific antibody ABX1446 failed to induce T-cell dependent cellular cytotoxicity (TDCC) against CD34 positive (CD34⁺) hematopoietic stem cells (HSCs), while Vibecotamab (CD123×CD3 bispecific) induced TDCC against CD34+ HSCs. For each treatment group, the bars from left to right represent 0.0006 μg/ml, 0.005 μg/ml, 0.04 μg/ml, 0.3 μg/ml and 2.5 μg/ml respectively.

FIG. 29 shows that ILT3×CD3 bispecific antibody ABX1446 failed to induce apoptosis against CD34 positive (CD34⁺) hematopoietic stem cells (HSCs), while Vibecotamab (CD123×CD3 bispecific) induced apoptosis against CD34+ HSCs. For each treatment group, the bars from left to right represent 0.001 μg/ml, 0.01 μg/ml, 0.1 μg/ml and 1.0 μg/ml respectively.

FIG. 30 shows that ILT3×CD3 bispecific antibody ABX1446 failed to induce apoptosis against non-monocytic KU812 basophils.

FIG. 31 shows that ILT3×CD3 bispecific antibody ABX1446 failed to induce apoptosis against non-monocytic LAMA84 basophils.

FIG. 32 shows T-cell dependent cellular cytotoxicity (TDCC) activity of various formats (FIG. 7) of ILT3×CD3 bispecific antibodies in MM1S cells, when expanded T cells were used as effectors. Anti-KLH represents the negative control and Blincyto (CD3×CD19 BiTe) represents the positive control.

FIG. 33 shows T-cell dependent cellular cytotoxicity (TDCC) activity of various formats (FIG. 7) of ILT3×CD3 bispecific antibodies in H929 cells, when expanded T cells were used as effectors. Anti-KLH represents the negative control and Blincyto (CD3×CD19 BiTe) represents the positive control.

FIG. 34 shows T-cell dependent cellular cytotoxicity (TDCC) activity of various formats (FIG. 7) of ILT3×CD3 bispecific antibodies in U226B1 cells, when expanded T cells were used as effectors. Anti-KLH represents the negative control and Blincyto (CD3×CD19 BiTe) represents the positive control.

FIG. 35 shows treatment schema and tumor burden quantification in a mouse AML model. The mice were irradiated 48 hours prior to MOLM13 or MV4; 11 cell injection. On day 2, expanded T cells were injected into the mice receiving MOLM13 cells or whole PBMCs were injected into mice receiving MV4; 11 cells. ABX1446 was then injected on day 7, day 14, day 21 and day 28. Tumor burden in the peripheral blood was then quantified utilizing FACS and the equation # of cells acquired/# of beads acquired×1000.

FIG. 36 shows that ABX1446 and ABX1520 reduced the circulating tumor burden in the MOLM13 mouse AML model. NTB represents non-tumor bearing mice, anti-KLH represents the negative control, hz45G10 represents an ILT3 antibody comprising an N297G substitution in the Fc region, IO-202 represents a monoclonal antibody that antagonizes ILT3, ABX1559 corresponds to an antibody lacking the ILT3 Fab binding region and thus represents an anti-CD3 only control, and Vibecotamab (CD123×CD3 bispecific) represents a positive control.

FIG. 37 shows that ABX1446 reduced the circulating tumor burden in the MV4; 11 mouse AML model at week 2. NTB represents non-tumor bearing mice, anti-KLH represents the negative control, and Vibecotamab (CD123×CD3 bispecific) represents a positive control (at increasing concentrations of 0.01 mpk, 0.1 mpk and 1 mpk, from left to right).

FIG. 38 shows that ABX1446 reduced the circulating tumor burden in the MV4; 11 mouse AML model at week 3. NTB represents non-tumor bearing mice, anti-KLH represents the negative control, and Vibecotamab (CD123×CD3 bispecific) represents a positive control (at increasing concentrations of 0.01 mpk, 0.1 mpk and 1 mpk, from left to right).

FIG. 39 shows treatment schema and tumor burden quantification in a mouse AML model. CD34⁺ hematopoietic stem cells (HSCs) were engrafted into mice 3 months prior to MV4; 11 cell injection. On day 7, day 14, day 21 and day 28, ABX1446 was injected. Tumor burden was then quantified utilizing FACS and the equation # of cells acquired/# of beads acquired×1000.

FIG. 40 shows that ABX1446 reduced the circulating tumor burden in CD34⁺ humanized mice. When dosed with anti-KLH (negative control), Vibecotamab (CD123×CD3 bispecific; positive control) and ABX1446 at 1 mpk, the number of circulating MV4; 11 cells per 1 μL of blood was greatly reduced.

FIG. 41 shows ABX1446 induced dose-dependent tumor cell depletion in primary M5 AML bone marrow cultures.

FIG. 42 shows ABX1446 induced dose-dependent T cell activation in primary M5 AML bone marrow cultures.

FIG. 43 shows ABX1446 induced dose-dependent tumor cell depletion in primary MM bone marrow cultures.

FIG. 44 shows ABX1446 induced dose-dependent T cell activation in primary MM bone marrow cultures.

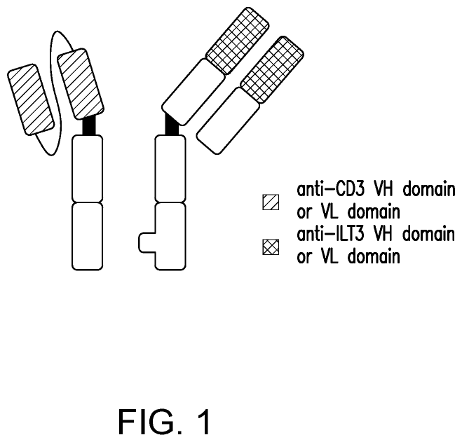

### 5. DETAILED DESCRIPTION

The present disclosure is based, in part, on the novel binding agents provided herein and surprising properties thereof.

Unless otherwise defined herein, technical and scientific terms used in the present description have the meanings that are commonly understood by those of ordinary skill in the art. Whenever appropriate, terms used in the singular will also include the plural and vice versa. In the event that any description of a term set forth conflicts with any document incorporated herein by reference, the description of the term set forth below shall control.

The term “binding agent” as used herein refers to a molecule that binds a specific antigen or target (e.g., ILT3 and/or CD3). A binding agent may comprise a protein, peptide, nucleic acid, carbohydrate, lipid, or small molecular weight compound. In some embodiments, a binding agent comprises a full-length antibody. In some embodiments, a binding agent is an antigen-binding fragment of an antibody. In some embodiments, a binding agent comprises an alternative protein scaffold or artificial scaffold (e.g., a non-immunoglobulin backbone). In some embodiments, a binding agent is a fusion protein comprising an antigen-binding site. In some embodiments, a binding agent is a bispecific molecule comprising at least two antigen-binding sites.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the binding molecules described herein terms such as “bind to,” “that specifically bind to,” and analogous terms are also used interchangeably herein and refer to binding molecules of antigen binding domains that specifically bind to an antigen, such as a polypeptide. A binding molecule or antigen binding domain that binds to or specifically binds to an antigen can be identified, for example, by immunoassays, Octet®, Biacore®, or other techniques known to those of skill in the art. In some embodiments, a binding molecule or antigen binding domain binds to or specifically binds to an antigen when it binds to an antigen with higher affinity than to any cross-reactive antigen as determined using experimental techniques, such as enzyme linked immunosorbent assay (ELISA). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of a binding molecule or antigen binding domain to a “non-target” protein is less than about 10% of the binding of the binding molecule or antigen binding domain to its particular target antigen, for example, as determined by fluorescence activated cell sorting (FACS) analysis. A binding molecule or antigen binding domain that binds to an antigen includes one that is capable of binding the antigen with sufficient affinity such that the binding molecule is useful, for example, as a therapeutic and/or diagnostic agent in targeting the antigen. In certain embodiments, a binding molecule or antigen binding domain that binds to an antigen has a dissociation constant (K_{D}) of less than or equal to 1 μM, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, a binding molecule or antigen binding domain binds to an epitope of an antigen that is conserved among the antigen from different species.

The term “antibody” is used herein in the broadest sense and encompasses various antibody structures, including but not limited to, an immunoglobulin molecule that recognizes and binds a target through at least one antigen-binding site, polyclonal antibodies, recombinant antibodies, monoclonal antibodies, chimeric antibodies, humanized antibodies, human antibodies, bispecific antibodies, multispecific antibodies, diabodies, tribodies, tetrabodies, single chain Fv (scFv) antibodies, and antibody fragments as long as they exhibit the desired antigen-binding activity.

A typical 4-chain antibody unit is a heterotetrametric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for u and & isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5th ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

In some embodiments, the Fab is a single chain Fab (scFab), wherein the heavy chain and light chain of the Fab is connected by a polypeptide linker.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody.

Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (a), delta (8), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. CDR1, CDR2 and CDR3 in VH domain are also referred to as HCDR1, HCDR2 and HCDR3, respectively. CDR1, CDR2 and CDR3 in VL domain are also referred to as LCDR1, LCDR2 and LCDR3, respectively. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat et al., supra; Nick Deschacht et al., J Immunol 2010; 184:5696-5704). Chothia refers instead to the location of the structural loops (see, e.g., Chothia and Lesk, J. Mol. Biol. 196:901-17 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., Dev. Comp. Immunol. 27 (1): 55-77 (2003)). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309:657-70 (2001). Correspondence between the numbering system, including, for example, the Kabat numbering and the IMGT unique numbering system, is well known to one skilled in the art (see, e.g., Kabat, supra; Chothia and Lesk, supra; Martin, supra, Lefranc et al., supra). The residues from each of these hypervariable regions or CDRs are exemplified in the table below.

Exemplary CDRs According to Various Numbering Systems

| Loop | Kabat | AbM | Chothia | Contact | IMGT |
|---|---|---|---|---|---|
| CDR L1 | L24--L34 | L24--L34 | L26--L32 or | L30--L36 | L27--L38 |
| | | | L24--L34 | | |
| CDR L2 | L50--L56 | L50--L56 | L50--L52 or | L46--L55 | L56--L65 |
| | | | L50--L56 | | |
| CDR L3 | L89--L97 | L89--L97 | L91--L96 or | L89--L96 | L105-L117 |
| | | | L89--L97 | | |
| CDR H1 | H31--H35B | H26--H35B | H26-- | H30--H35B | H27--H38 |
| | (Kabat | | H32 . . . 34 | | |
| | Numbering) | | | | |
| CDR H1 | H31--H35 | H26--H35 | H26--H32 | H30--H35 | |
| | (Chothia | | | | |
| | Numbering) | | | | |
| CDR H2 | H50--H65 | H50--H58 | H53--H55 or | H47--H58 | H56--H65 |
| | | | H52--H56 | | |
| CDR H3 | H95--H102 | H95--H102 | H96--H101 or | H93--H101 | H105-H117 |
| | | | H95--H102 | | |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, Chothia, or Contact method. In other cases, the particular amino acid sequence of a CDR is given. It should be noted CDR regions may also be defined by a combination of various numbering systems, e.g., a combination of Kabat and Chothia numbering systems, or a combination of Kabat and IMGT numbering systems. Therefore, the term such as “a CDR1 as set forth in a specific VH” includes any CDR1 as defined by the exemplary CDR numbering systems described above, but is not limited thereby. Once a variable region (e.g., a VH or VL) is given, those skilled in the art would understand that CDRs within the region can be defined by different numbering systems or combinations thereof.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

The term “antibody fragment” as used herein refers to a molecule other than an intact antibody that comprises a portion of an antibody and generally an antigen-binding site. Examples of antibody fragments include, but are not limited to, Fab, Fab′, F(ab′)₂, Fv, single chain antibody molecules, scFv, sc (Fv)₂, disulfide-linked scFv (dsscFv), diabodies, tribodies, tetrabodies, minibodies, dual variable domain antibodies (DVD), single variable domain antibodies (e.g., camelid antibodies), and multispecific antibodies formed from antigen-binding antibody fragments.

The term “monoclonal antibody” as used herein refers to a substantially homogenous antibody population involved in the highly specific recognition and binding of a single antigenic determinant or epitope. The term “monoclonal antibody” encompasses intact and full-length antibodies as well as antibody fragments (e.g., Fab, Fab′, F(ab′)₂, Fv), single chain antibodies, scFv, fusion proteins comprising an antigen-binding antibody fragment, and any other modified immunoglobulin molecule comprising at least one antigen-binding site. Furthermore, “monoclonal antibody” refers to such antibodies made by any number of techniques, including but not limited to, hybridoma production, phage library display, recombinant expression, and transgenic animals.

The terms “epitope” and “antigenic determinant” are used interchangeably herein and refer to that portion of an antigen or target capable of being recognized and bound by a particular antibody. When the antigen or target is a polypeptide, epitopes can be formed both from contiguous amino acids and noncontiguous amino acids juxtaposed by tertiary folding of the protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation. Epitopes can be predicted using any one of a large number of publicly available bioinformatic software tools. X-ray crystallography may be used to characterize an epitope on a target protein by analyzing the amino acid residue interactions of an antigen/antibody complex.

The term “chimeric antibody” refers to an antibody in which a portion of the heavy and/or light chain is derived from a first source or species, while the remainder of the heavy and/or light chain is derived from a different source or species.

The term “humanized antibody” as used herein refers to an antibody that comprises a human heavy chain variable region and a light chain variable region wherein the native CDR amino acid residues are replaced by residues from corresponding CDRs from a non-human antibody (e.g., mouse, rat, rabbit, or non-human primate), wherein the non-human antibody has the desired specificity, affinity, and/or activity. In some embodiments, one or more framework region amino acid residues of the human heavy chain or light chain variable regions are replaced by corresponding residues from the non-human antibody. Furthermore, humanized antibodies can comprise amino acid residues that are not found in the human antibody or in the non-human antibody. In some embodiments, these modifications are made to further refine and/or optimize antibody characteristics. In some embodiments, the humanized antibody comprises at least a portion of a human immunoglobulin constant region (e.g., CH1, CH2, CH3, Fc, and/or hinge region).

The term “human antibody” as used herein refers to an antibody that possesses an amino acid sequence that corresponds to an antibody produced by a human and/or an antibody that has been made using any of the techniques that are known to those of skill in the art for making human antibodies. These techniques include, but not limited to, phage display libraries, yeast display libraries, transgenic animals, recombinant protein production, and B-cell hybridoma technology.

The term “specifically binds” as used herein refers to an agent that interacts more frequently, more rapidly, with greater duration, with greater affinity, or with some combination of the above to a particular antigen, epitope, protein, or target molecule than with alternative substances. The terms “specifically binds” and “binds” are used interchangeably in some embodiments. A binding agent that specifically binds an antigen can be identified, for example, by immunoassays, ELISAs, surface plasmon resonance (SPR), or other techniques known to those of skill in the art. In some embodiments, an agent that specifically binds an antigen (e.g., human ILT3 or CD3) can bind related antigens (e.g., cyno ILT3 or CD3). Generally, a binding agent that specifically binds an antigen will bind the target antigen at a higher affinity than its affinity for a different antigen. The different antigen can be a related antigen. In some embodiments, a binding agent that specifically binds an antigen can bind the target antigen with an affinity that is at least 20 times greater, at least 30 times greater, at least 40 times greater, at least 50 times greater, at least 60 times greater, at least 70 times greater, at least 80 times greater, at least 90 times greater, or at least 100 times greater, than its affinity for a different antigen. In some embodiments, a binding agent that specifically binds a particular antigen binds a different antigen at such a low affinity that binding cannot be detected using an assay described herein or otherwise known in the art. In some embodiments, affinity is measured using SPR technology in a Biacore system as described herein or as known to those of skill in the art.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies, the term “polypeptide” encompasses polypeptides as a single chain and polypeptides of two or more associated chains.

The terms “polynucleotide” and “nucleic acid” and “nucleic acid molecule” are used interchangeably herein and refer to polymers of nucleotides of any length, and include DNA and

RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the disclosure are substantially identical, meaning they have at least 70%, at least 75%, at least 80%, at least 85%, at least 90%, and in some embodiments at least 95%, 96%, 97%, 98%, 99% nucleotide or amino acid identity, when compared and aligned for maximum correspondence, as measured using a sequence comparison algorithm or by visual inspection. In some embodiments, identity exists over a region of the sequences that is at least about 10, at least about 20, at least about 20-40, at least about 40-60, at least about 60-80 nucleotides or amino acids in length, or any integral value there between. In some embodiments, identity exists over a longer region than 60-80 nucleotides or amino acids, such as at least about 80-100 nucleotides or amino acids, and in some embodiments the sequences are substantially identical over the full length of the sequences being compared, for example, (i) the coding region of a nucleotide sequence or (ii) an amino acid sequence.

The term “vector” as used herein means a construct that is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

The term “isolated” as used herein refers to a polypeptide, soluble protein, antibody, polynucleotide, vector, cell, or composition that is in a form not found in nature. An “isolated” antibody is substantially free of material from the cellular source from which it is derived. In some embodiments, isolated polypeptides, soluble proteins, antibodies, polynucleotides, vectors, cells, or compositions are those that have been purified to a degree that they are no longer in a form in which they are found in nature. In some embodiments, a polypeptide, soluble protein, antibody, polynucleotide, vector, cell, or composition that is isolated is substantially pure. A polypeptide, soluble protein, antibody, polynucleotide, vector, cell, or composition can be isolated from a natural source (e.g., tissue) or from a source such as an engineered cell line.

The term “substantially pure” as used herein refers to material that is at least 50% pure (i.e., free from contaminants), at least 90% pure, at least 95% pure, at least 98% pure, or at least 99% pure.

The term “subject” refers to any animal (e.g., a mammal), including, but not limited to, humans, non-human primates, canines, felines, rabbits, rodents, and the like.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. The term “pharmaceutically acceptable” as used herein refers to a substance approved or approvable by a regulatory agency or listed in the U.S. Pharmacopeia, European Pharmacopeia, or other generally recognized pharmacopeia for use in animals, including humans.

The terms “pharmaceutically acceptable excipient, carrier, or adjuvant” or “acceptable pharmaceutical carrier” as used herein refer to an excipient, carrier, or adjuvant that can be administered to a subject, together with at least one therapeutic agent, and that is generally safe, non-toxic, and has no effect on the pharmacological activity of the therapeutic agent. In general, those of skill in the art and government agencies consider a pharmaceutically acceptable excipient, carrier, or adjuvant to be an inactive ingredient of any formulation or any pharmaceutical composition.

The term “pharmaceutical composition” or “pharmaceutical formulation” as used herein refers to a preparation that is in such form as to permit the biological activity of the binding agent to be effective. A pharmaceutical formulation or composition generally comprises additional components, such as a pharmaceutically acceptable excipient, carrier, adjuvant, buffers, etc.

The term “effective amount” or “therapeutically effective amount” as used herein refers to the amount of an agent that is sufficient to reduce and/or ameliorate the severity and/or duration of (i) a disease, disorder or condition in a subject, and/or (ii) a symptom in a subject. The term also encompasses an amount of an agent necessary for the (i) reduction or amelioration of the advancement or progression of a given disease, disorder, or condition, (ii) reduction or amelioration of the recurrence, development, or onset of a given disease, disorder, or condition, and/or (iii) the improvement or enhancement of the prophylactic or therapeutic effect(s) of another agent or therapy (e.g., an agent other than the binding agents provided herein).

The term “treat” or “treatment” or “treating” or “to treat” or “alleviate” or alleviation” or “alleviating” or “to alleviate” as used herein refers to therapeutic measures that aim to cure, slow down, lessen symptoms of, and/or halt progression of a pathologic condition or disorder. Thus, those in need of treatment include those already with the disorder.

The term “immune response” as used herein includes responses from both the innate immune system and the adaptive immune system. It includes both cell-mediated and/or humoral immune responses. It includes both T-cell and B-cell responses, as well as responses from other cells of the immune system such as natural killer (NK) cells, monocytes, macrophages, dendritic cells, etc.

As used herein, reference to “about” or “approximately” a value or parameter includes (and describes) embodiments that are directed to that value or parameter. For example, a description referring to “about X” includes description of “X”.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C, A or B; B or C; A and C; A and B; B and C; A (alone); B (alone); and C (alone).

### 5.1 ILT3 Binding Regions

The binding agents provided here comprise a region that binds ILT3 (e.g., human ILT3), and thus the present binding agents are ILT3 binding agents.

Amino acid (aa) sequences for human ILT3 (UniProtKB No. Q8NHJ6) and cynomolgus monkey (“cyno”) ILT3 (NCBI Ref No. XP_015297198) are known. ILT3 is a single pass type I transmembrane protein with a predicted molecular weight of approximately 47 kDa. ILT3 has been observed to be predominantly expressed on myeloid antigen presenting cells, such as normal monocytes, macrophages, and dendritic cells. ILT3 is characterized by an extracellular domain comprising two Ig-like C2 type domains, a transmembrane domain, and a long cytoplasmic domain containing 3 ITIM domains (see, e.g., Cella et al., 1997, J. Exp. Med., 185:1743-1751). The two Ig-like C2-type domains may be referred to herein as Domain 1 (D1) and Domain 2 (D2). D1 is situated at the N-terminal portion of the protein and D2 is situated closest to the transmembrane region. As characterized within UniProtKB, human ILT3 is a protein of 448 amino acids (aa)—the signal sequence is aa 1-21, the extracellular domain is aa 22-259, the transmembrane region is aa 260-280, and the cytoplasmic domain is aa 281-448. Within the extracellular domain, D1 is aa 27-188, D2 is aa 124-218, and the “stem region” is aa 219-259. Within the cytoplasmic domain, ITIMs are aa 358-363, 410-415, and 440-445.

The present disclosure provides agents (e.g., bispecific antibodies) that bind ILT3. In some embodiments, the ILT3 binding agent binds a human ILT3 or a fragment thereof.

In some embodiments, the ILT3 binding region in the present binding agent is an antibody or a binding domain derived from an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In some embodiments, the antibody is an IgG antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody. In some embodiments, the antibody comprises an IgG heavy chain. In some embodiments, the antibody comprises an IgG1 heavy chain. In some embodiments, the antibody comprises an IgG2 heavy chain. In some embodiments, the antibody comprises an IgG4 heavy chain. In some embodiments, the antibody comprises a kappa light chain. In some embodiments, the antibody comprises a kappa light chain constant region. In some embodiments, the antibody comprises a lambda light chain. In some embodiments, the antibody comprises a lambda light chain constant region. In some embodiments, the antibody is an antibody fragment comprising an antigen-binding site. In some embodiments, the antibody is an scFv. In some embodiments, the antibody is a disulfide-linked scFv. In some embodiments, the antibody is a disulfide-linked sc (Fv)₂. In some embodiments, the antibody is a Fab, Fab′, or a F (ab)₂ antibody. In some embodiments, the antibody is a single chain Fab (scFab). In some embodiments, the antibody is a diabody. In some embodiments, the antibody is a nanobody. In some embodiments, the antibody is a monospecific antibody. In some embodiments, the antibody is a bispecific antibody. In some embodiments, the antibody is a monovalent antibody. In some embodiments, the antibody is a multivalent antibody. In some embodiments, the antibody is a bivalent antibody. In some embodiments, the antibody is a tetravalent antibody.

In some embodiments, the ILT3 binding region is derived from a monoclonal antibody. Monoclonal antibodies can be prepared by any method known to those of skill in the art. In some embodiments, monoclonal antibodies are prepared using hybridoma methods known to one of skill in the art. For example, using a hybridoma method, a mouse, rat, rabbit, hamster, or other appropriate host animal, is immunized as described above. In some embodiments, lymphocytes are immunized in vitro. In some embodiments, the immunizing antigen is a human protein or a fragment thereof. In some embodiments, the immunizing antigen is a mouse protein or a fragment thereof. In some embodiments, the immunizing antigen is a cyno protein or a fragment thereof.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed specifically against a chosen antigen can be identified by a variety of methods including, but not limited to, immunoprecipitation, immunoblotting, and in vitro binding assays (e.g., flow cytometry, FACS, ELISA, SPR (e.g., Biacore), and radioimmunoassay). Once hybridoma cells that produce antibodies of the desired specificity, affinity, and/or activity are identified, the clones may be subcloned by limiting dilution techniques. In some embodiments, high-throughput methods are used to distribute single cell hybridoma cells into plates. The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In some embodiments, monoclonal antibodies are made using recombinant DNA techniques as known to one skilled in the art. For example, the polynucleotides encoding an antibody are isolated from mature B-cells or hybridoma cells, such as by RT-PCR using oligonucleotide primers that specifically amplify the genes encoding the heavy and light chains of the antibody, and their sequence is determined using standard techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins.

In some embodiments, recombinant monoclonal antibodies are isolated from phage display libraries expressing variable domains or CDRs of a desired species. Screening of phage libraries can be accomplished by various techniques known in the art.

In some embodiments, a monoclonal antibody is modified by using recombinant DNA technology to generate alternative antibodies. In some embodiments, the constant domains of the light chain and heavy chain of a mouse monoclonal antibody are substituted for constant regions of a human antibody to generate a chimeric antibody. In some embodiments, the constant regions are truncated or removed to generate a desired antibody fragment of a monoclonal antibody. In some embodiments, site-directed or high-density mutagenesis of the variable region(s) is used to optimize specificity and affinity of a monoclonal antibody.

In some embodiments, the ILT3 binding region is derived from a humanized antibody. Various methods for generating humanized antibodies are known in the art. In some embodiments, a humanized antibody comprises one or more amino acid residues that have been introduced into it from a source that is non-human. In some embodiments, humanization is performed by substituting one or more non-human CDR sequences for the corresponding CDR sequences of a human antibody. In some embodiments, the humanized antibodies are constructed by substituting all six CDRs of a non-human antibody (e.g., a mouse antibody) for the corresponding CDRs of a human antibody.

The choice of which human heavy chain variable region and/or light chain variable region to use for generating humanized antibodies can be made based on a variety of factors and by a variety of methods known in the art. In some embodiments, the “best-fit” method is used where the sequence of the variable region of a non-human (e.g., rodent) antibody is screened against the entire library of known human variable region sequences. The human sequence that is most similar to that of the non-human (e.g., rodent) sequence is selected as the human variable region framework for the humanized antibody. In some embodiments, a particular variable region framework derived from a consensus sequence of all human antibodies of a particular subgroup of light or heavy chains is selected as the variable region framework. In some embodiments, the variable region framework sequence is derived from the consensus sequences of the most abundant human subclasses. In some embodiments, human germline genes are used as the source of the variable region framework sequences.

Other methods for humanization include, but are not limited to, a method called “superhumanization” which is described as the direct transfer of CDRs to a human germline framework, a method termed Human String Content (HSC) which is based on a metric of “antibody humanness”, methods based on generation of large libraries of humanized variants (including phage, ribosomal, and yeast display libraries), and methods based on framework region shuffling.

In some embodiments, the ILT3 binding region is derived from a human antibody. Human antibodies can be prepared using various techniques known in the art. In some embodiments, human antibodies are generated from immortalized human B lymphocytes immunized in vitro. In some embodiments, human antibodies are generated from lymphocytes isolated from an immunized individual. In any case, cells that produce an antibody directed against a target antigen can be generated and isolated. In some embodiments, a human antibody is selected from a phage library, where that phage library expresses human antibodies. Alternatively, phage display technology may be used to produce human antibodies and antibody fragments in vitro, from immunoglobulin variable region gene repertoires from unimmunized human donors. Techniques for the generation and use of antibody phage libraries are well known in the art. Once antibodies are identified, affinity maturation strategies known in the art, including but not limited to, chain shuffling and site-directed mutagenesis, may be employed to generate higher affinity human antibodies. In some embodiments, human antibodies are produced in transgenic mice that contain human immunoglobulin loci. Upon immunization these mice are capable of producing the full repertoire of human antibodies in the absence of endogenous immunoglobulin production.

In some embodiments, the ILT3 binding region is an antibody fragment. As used herein, the term “antibody fragment” refers to a molecule other than an intact antibody that comprises a portion of an antibody and generally an antigen-binding site. Examples of antibody fragments include, but are not limited to, Fab, Fab′, F(ab′)₂, Fv, single chain antibody molecules (e.g., scFv), disulfide-linked scFv (dsscFv), nanobodies, diabodies, tribodies, tetrabodies, minibodies, dual variable domain antibodies (DVD), single variable domain antibodies (e.g., camelid antibodies), and multispecific antibodies formed from antibody fragments.

In some specific embodiments, the ILT3 binding region comprises an scFv that binds ILT3. In some specific embodiments, the ILT3 binding region comprises one or more Fabs that bind ILT3. In some specific embodiments, the ILT3 binding region comprises a Fab. In other specific embodiments, the ILT3 binding region comprises two Fabs. In other specific embodiments, the ILT3 binding region comprises two Fabs in tandem.

Antibody fragments can be made by various techniques, including but not limited to proteolytic digestion of an intact antibody. The antibody fragments described herein can be produced using recombinant technologies known in the art (e.g., E. coli or phage expression).

In some embodiments, the ILT3 binding region provided herein binds to ILT3 (e.g., human ILT3) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸ M to 10⁻¹³ M, e.g., from 10⁻⁹M to 10⁻¹³ M). In some embodiments, the ILT3 binding region provided herein binds to ILT3 (e.g., human ILT3) with a dissociation constant of ≤0.1 nM. In some embodiments, the ILT3 binding region provided herein binds to ILT3 (e.g., human ILT3) with a dissociation constant of ≤0.2 nM. In some embodiments, the ILT3 binding region provided herein binds to ILT3 (e.g., human ILT3) with a dissociation constant of ≤0.3 nM. In some embodiments, the ILT3 binding region provided herein binds to ILT3 (e.g., human ILT3) with a dissociation constant of ≤0.8 nM. In some embodiments, the ILT3 binding region provided herein binds to ILT3 (e.g., human ILT3) with a dissociation constant of ≤3 nM. In some embodiments, the ILT3 binding region provided herein binds to ILT3 (e.g., human ILT3) with a dissociation constant of ≤9 nM. A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kₒₙ” may also be determined with the same biolayer interferometry (BLI) or surface plasmon resonance (SPR) techniques described above using, for example, the Octet®Red96, the Biacore®TM-3000, or the Biacore®TM-8000 system.

Any ILT3 binding agents (e.g., anti-ILT3 antibodies) known in the art can be used for deriving the ILT3 binding region disclosed herein. In certain embodiments, the ILT3 binding region disclosed herein is derived from any of the ILT3 antibodies disclosed in International Publication No. WO2021/183839, the content of which is incorporated by reference herein. For example, the ILT3 binding region disclosed herein is derived from H7K3 or its variants disclosed in WO2021/183839. In certain embodiments, the H7K3 variant comprises a VH variant selected from the group consisting of H7m1, H7m2, H7m3, and H7m4, and/or comprises a VL variant selected from the group consisting of K3m1, K3m2, K3m3, K3m4, K3m5, K3m6, K3m7, and K3m8 as disclosed in WO2021/183839. The amino acid sequences of CDRs, VL and VH of H7K3 and its variants are disclosed, for example, in Table 1 and paragraph of WO2021/183839. In certain embodiments, the ILT3 binding region disclosed herein is derived from any anti-ILT3 antibodies described in any of the following patent publications: US20190153093, WO2020056077, WO2021183839, US20200031926, US20210221887, US20150110714, US20200031926, US20190241655, WO2020180789, and WO2020056077, the content of each of which is incorporated by reference herein.

In some embodiments, the ILT3 binding region provided herein is derived from an antibody in International Publication No. WO 2021/127200, the content of which incorporated by reference herein. In some embodiments, the ILT3 binding region is any one of those in Tables 1-8.

In some embodiments, the ILT3 binding region provided herein comprises one or more CDR sequences of the amino acid sequence set forth in any one of SEQ ID NO: 17, SEQ ID NO:18, SEQ ID NO:37, SEQ ID NO:38, SEQ ID NO:55, SEQ ID NO:56, SEQ ID NO: 73, SEQ ID NO:74, SEQ ID NO:91, SEQ ID NO:92, SEQ ID NO: 109, SEQ ID NO: 110, SEQ ID NO: 127, SEQ ID NO: 128, SEQ ID NO:145, and SEQ ID NO:146. CDR sequences can be determined and defined according to any well-known numbering systems. In some embodiments, the CDRs are determined and defined according to IMGT numbering. In some embodiments, the CDRs are determined and defined according to Kabat numbering. In some embodiments, the CDRs are determined and defined according to AbM numbering. In other embodiments, the CDRs are determined and defined according to Chothia numbering. In other embodiments, the CDRs are determined and defined according to Contact numbering. In some embodiments, the ILT3 binding region is humanized. In some embodiments, the ILT3 binding region comprises an acceptor human framework, e.g., a human immunoglobulin framework or a human consensus framework.

In some embodiments, the ILT3 binding region provided herein comprises a HCDR1, a HCDR2, and a HCDR3 of the amino acid sequence set forth in SEQ ID NO:17. In some embodiments, the ILT3 binding region provided herein comprises a LCDR1, a LCDR2, and a LCDR3 of the amino acid sequence set forth in SEQ ID NO:18. In some embodiments, the ILT3 binding region provided herein comprises a HCDR1, a HCDR2, and a HCDR3 of the amino acid sequence set forth in SEQ ID NO:17, and a LCDR1, a LCDR2, and a LCDR3 of the amino acid sequence set forth in SEQ ID NO:18.

In some embodiments, the ILT3 binding region provided herein comprises a HCDR1, a HCDR2, and a HCDR3 of the amino acid sequence set forth in SEQ ID NO:37. In some embodiments, the ILT3 binding region provided herein comprises a LCDR1, a LCDR2, and a LCDR3 of the amino acid sequence set forth in SEQ ID NO:38. In some embodiments, the ILT3 binding region provided herein comprises a HCDR1, a HCDR2, and a HCDR3 of the amino acid sequence set forth in SEQ ID NO:37, and a LCDR1, a LCDR2, and a LCDR3 of the amino acid sequence set forth in SEQ ID NO:38.

In some embodiments, the ILT3 binding region provided herein comprises a HCDR1, a HCDR2, and a HCDR3 of the amino acid sequence set forth in SEQ ID NO:55. In some embodiments, the ILT3 binding region provided herein comprises a LCDR1, a LCDR2, and a LCDR3 of the amino acid sequence set forth in SEQ ID NO:56. In some embodiments, the ILT3 binding region provided herein comprises a HCDR1, a HCDR2, and a HCDR3 of the amino acid sequence set forth in SEQ ID NO:55, and a LCDR1, a LCDR2, and a LCDR3 of the amino acid sequence set forth in SEQ ID NO:56.

In some embodiments, the ILT3 binding region provided herein comprises a HCDR1, a HCDR2, and a HCDR3 of the amino acid sequence set forth in SEQ ID NO:73. In some embodiments, the ILT3 binding region provided herein comprises a LCDR1, a LCDR2, and a LCDR3 of the amino acid sequence set forth in SEQ ID NO:74. In some embodiments, the ILT3 binding region provided herein comprises a HCDR1, a HCDR2, and a HCDR3 of the amino acid sequence set forth in SEQ ID NO:73, and a LCDR1, a LCDR2, and a LCDR3 of the amino acid sequence set forth in SEQ ID NO:74.

In some embodiments, the ILT3 binding region provided herein comprises a HCDR1, a HCDR2, and a HCDR3 of the amino acid sequence set forth in SEQ ID NO:91. In some embodiments, the ILT3 binding region provided herein comprises a LCDR1, a LCDR2, and a LCDR3 of the amino acid sequence set forth in SEQ ID NO:92. In some embodiments, the ILT3 binding region provided herein comprises a HCDR1, a HCDR2, and a HCDR3 of the amino acid sequence set forth in SEQ ID NO:91, and a LCDR1, a LCDR2, and a LCDR3 of the amino acid sequence set forth in SEQ ID NO:92.

In some embodiments, the ILT3 binding region provided herein comprises a HCDR1, a HCDR2, and a HCDR3 of the amino acid sequence set forth in SEQ ID NO: 109. In some embodiments, the ILT3 binding region provided herein comprises a LCDR1, a LCDR2, and a LCDR3 of the amino acid sequence set forth in SEQ ID NO:110. In some embodiments, the ILT3 binding region provided herein comprises a HCDR1, a HCDR2, and a HCDR3 of the amino acid sequence set forth in SEQ ID NO:109, and a LCDR1, a LCDR2, and a LCDR3 of the amino acid sequence set forth in SEQ ID NO: 110.

In some embodiments, the ILT3 binding region provided herein comprises a HCDR1, a HCDR2, and a HCDR3 of the amino acid sequence set forth in SEQ ID NO:127. In some embodiments, the ILT3 binding region provided herein comprises a LCDR1, a LCDR2, and a LCDR3 of the amino acid sequence set forth in SEQ ID NO:128. In some embodiments, the ILT3 binding region provided herein comprises a HCDR1, a HCDR2, and a HCDR3 of the amino acid sequence set forth in SEQ ID NO: 127, and a LCDR1, a LCDR2, and a LCDR3 of the amino acid sequence set forth in SEQ ID NO:128.

In some embodiments, the ILT3 binding region provided herein comprises a HCDR1, a HCDR2, and a HCDR3 of the amino acid sequence set forth in SEQ ID NO:145. In some embodiments, the ILT3 binding region provided herein comprises a LCDR1, a LCDR2, and a LCDR3 of the amino acid sequence set forth in SEQ ID NO:146. In some embodiments, the ILT3 binding region provided herein comprises a HCDR1, a HCDR2, and a HCDR3 of the amino acid sequence set forth in SEQ ID NO:145, and a LCDR1, a LCDR2, and a LCDR3 of the amino acid sequence set forth in SEQ ID NO: 146.

CDR sequences can be determined according to well-known numbering systems or a combination thereof. In some embodiments, the CDRs are defined according to IMGT numbering. In some embodiments, the CDRs are defined according to Kabat numbering. In some embodiments, the CDRs are defined according to AbM numbering. In other embodiments, the CDRs are defined according to Chothia numbering. In other embodiments, the CDRs are defined according to Contact numbering.

In other embodiments, the ILT3 binding region comprises a HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any one of SEQ ID NOs: 1, 7, 10 and 11; (ii) a HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any one of SEQ ID NOs: 2, 8, 9, and 12, (iii) a HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO:3 or 13; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 4 or 14; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 5 or 15; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs: 6 or 16. In some embodiments, the ILT3 binding region is humanized. In some embodiments, the ILT3 binding region comprises an acceptor human framework, e.g., a human immunoglobulin framework or a human consensus framework.

In some specific embodiments, the ILT3 binding region provided herein comprises one or more CDRs in Table 1.

In some embodiments, the ILT3 binding region provided herein comprises a HCDR1 comprising the amino acid sequence of any one of SEQ ID NOs: 1, 7, 10 and 11; (ii) a HCDR2 comprising the amino acid sequence of any one of SEQ ID NOs: 2, 8, 9, and 12, (iii) a HCDR3 comprising the amino acid sequence of SEQ ID NOs: 3 or 13; (iv) a LCDR1 comprising the amino acid sequence of SEQ ID NOs: 4 or 14; (v) a LCDR2 comprising the amino acid sequence of SEQ ID NOs: 5 or 15; and/or (vi) a LCDR3 comprising the amino acid sequence of SEQ ID NOs: 6 or 16.

In some specific embodiments, in the ILT3 binding region provided herein, the HCDR1 comprises the amino acid sequence of SEQ ID NO:1, the HCDR2 comprises the amino acid sequence of SEQ ID NO:2, the HCDR3 comprises the amino acid sequence of SEQ ID NO:3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO:6.

In some specific embodiments, in the ILT3 binding region provided herein, the HCDR1 comprises the amino acid sequence of SEQ ID NO:7, the HCDR2 comprises the amino acid sequence of SEQ ID NO:8, the HCDR3 comprises the amino acid sequence of SEQ ID NO:3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO:6.

In some specific embodiments, in the ILT3 binding region provided herein, the HCDR1 comprises the amino acid sequence of SEQ ID NO:1, the HCDR2 comprises the amino acid sequence of SEQ ID NO:9, the HCDR3 comprises the amino acid sequence of SEQ ID NO:3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO:6.

In some specific embodiments, in the ILT3 binding region provided herein, the HCDR1 comprises the amino acid sequence of SEQ ID NO:10, the HCDR2 comprises the amino acid sequence of SEQ ID NO:2, the HCDR3 comprises the amino acid sequence of SEQ ID NO:3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO:6.

In some specific embodiments, in the ILT3 binding region provided herein, the HCDR1 comprises the amino acid sequence of SEQ ID NO:11, the HCDR2 comprises the amino acid sequence of SEQ ID NO: 12, the HCDR3 comprises the amino acid sequence of SEQ ID NO:13, the LCDR1 comprises the amino acid sequence of SEQ ID NO:14, the LCDR2 comprises the amino acid sequence of SEQ ID NO: 15, and the LCDR3 comprises the amino acid sequence of SEQ ID NO: 16.

In other embodiments, the ILT3 binding region comprises a HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any one of SEQ ID NOs: 21, 27, 30 and 31; (ii) a HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any one of SEQ ID NOs: 22, 28, 29, and 32, (iii) a HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 23 or 33; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 24 or 34; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 25 or 35; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs: 26 or 36. In some embodiments, the ILT3 binding region is humanized. In some embodiments, the ILT3 binding region comprises an acceptor human framework, e.g., a human immunoglobulin framework or a human consensus framework.

In some specific embodiments, the ILT3 binding region provided herein comprises one or more CDRs in Table 2.

In some embodiments, the ILT3 binding region provided herein comprises a HCDR1 comprising the amino acid sequence of any one of SEQ ID NOs: 21, 27, 30 and 31; (ii) a HCDR2 comprising the amino acid sequence of any one of SEQ ID NOs: 22, 28, 29, and 32, (iii) a HCDR3 comprising the amino acid sequence of SEQ ID NOs: 23 or 33; (iv) a LCDR1 comprising the amino acid sequence of SEQ ID NOs: 24 or 34; (v) a LCDR2 comprising the amino acid sequence of SEQ ID NOs: 25 or 35; and/or (vi) a LCDR3 comprising the amino acid sequence of SEQ ID NOs: 26 or 36.

In some specific embodiments, in the ILT3 binding region provided herein, the HCDR1 comprises the amino acid sequence of SEQ ID NO:21, the HCDR2 comprises the amino acid sequence of SEQ ID NO:22, the HCDR3 comprises the amino acid sequence of SEQ ID NO:23, the LCDR1 comprises the amino acid sequence of SEQ ID NO:24, the LCDR2 comprises the amino acid sequence of SEQ ID NO:25, and the LCDR3 comprises the amino acid sequence of SEQ ID NO: 26.

In some specific embodiments, in the ILT3 binding region provided herein, the HCDR1 comprises the amino acid sequence of SEQ ID NO:27, the HCDR2 comprises the amino acid sequence of SEQ ID NO:28, the HCDR3 comprises the amino acid sequence of SEQ ID NO:23, the LCDR1 comprises the amino acid sequence of SEQ ID NO:24, the LCDR2 comprises the amino acid sequence of SEQ ID NO:25, and the LCDR3 comprises the amino acid sequence of SEQ ID NO: 26.

In some specific embodiments, in the ILT3 binding region provided herein, the HCDR1 comprises the amino acid sequence of SEQ ID NO:21, the HCDR2 comprises the amino acid sequence of SEQ ID NO:29, the HCDR3 comprises the amino acid sequence of SEQ ID NO:23, the LCDR1 comprises the amino acid sequence of SEQ ID NO:24, the LCDR2 comprises the amino acid sequence of SEQ ID NO:25, and the LCDR3 comprises the amino acid sequence of SEQ ID NO: 26.

In some specific embodiments, in the ILT3 binding region provided herein, the HCDR1 comprises the amino acid sequence of SEQ ID NO:30, the HCDR2 comprises the amino acid sequence of SEQ ID NO:22, the HCDR3 comprises the amino acid sequence of SEQ ID NO:23, the LCDR1 comprises the amino acid sequence of SEQ ID NO:24, the LCDR2 comprises the amino acid sequence of SEQ ID NO:25, and the LCDR3 comprises the amino acid sequence of SEQ ID NO: 26.

In some specific embodiments, in the ILT3 binding region provided herein, the HCDR1 comprises the amino acid sequence of SEQ ID NO:31, the HCDR2 comprises the amino acid sequence of SEQ ID NO:32, the HCDR3 comprises the amino acid sequence of SEQ ID NO:33, the LCDR1 comprises the amino acid sequence of SEQ ID NO:34, the LCDR2 comprises the amino acid sequence of SEQ ID NO:35, and the LCDR3 comprises the amino acid sequence of SEQ ID NO: 36.

In other embodiments, the ILT3 binding region comprises a HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any one of SEQ ID NOs: 39, 45, 48 and 49; (ii) a HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any one of SEQ ID NOs: 40, 46, 47 and 50, (iii) a HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 41 or 51; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 42 or 52; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 43 or 53; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs: 44 or 54. In some embodiments, the ILT3 binding region is humanized. In some embodiments, the ILT3 binding region comprises an acceptor human framework, e.g., a human immunoglobulin framework or a human consensus framework.

In some specific embodiments, the ILT3 binding region provided herein comprises one or more CDRs in Table 3.

In some embodiments, the ILT3 binding region provided herein comprises a HCDR1 comprising the amino acid sequence of any one of SEQ ID NOs: 39, 45, 48 and 49; (ii) a HCDR2 comprising the amino acid sequence of any one of SEQ ID NOs: 40, 46, 47 and 50, (iii) a HCDR3 comprising the amino acid sequence of SEQ ID NOs: 41 or 51; (iv) a LCDR1 comprising the amino acid sequence of SEQ ID NOs: 42 or 52; (v) a LCDR2 comprising the amino acid sequence of SEQ ID NOs: 43 or 53; and/or (vi) a LCDR3 comprising the amino acid sequence of SEQ ID NOs: 44 or 54.

In some specific embodiments, in the ILT3 binding region provided herein, the HCDR1 comprises the amino acid sequence of SEQ ID NO:39, the HCDR2 comprises the amino acid sequence of SEQ ID NO:40, the HCDR3 comprises the amino acid sequence of SEQ ID NO:41, the LCDR1 comprises the amino acid sequence of SEQ ID NO:42, the LCDR2 comprises the amino acid sequence of SEQ ID NO:43, and the LCDR3 comprises the amino acid sequence of SEQ ID NO: 44.

In some specific embodiments, in the ILT3 binding region provided herein, the HCDR1 comprises the amino acid sequence of SEQ ID NO:45, the HCDR2 comprises the amino acid sequence of SEQ ID NO:46, the HCDR3 comprises the amino acid sequence of SEQ ID NO:41, the LCDR1 comprises the amino acid sequence of SEQ ID NO:42, the LCDR2 comprises the amino acid sequence of SEQ ID NO:43, and the LCDR3 comprises the amino acid sequence of SEQ ID NO: 44.

In some specific embodiments, in the ILT3 binding region provided herein, the HCDR1 comprises the amino acid sequence of SEQ ID NO:39, the HCDR2 comprises the amino acid sequence of SEQ ID NO:47, the HCDR3 comprises the amino acid sequence of SEQ ID NO:41, the LCDR1 comprises the amino acid sequence of SEQ ID NO:42, the LCDR2 comprises the amino acid sequence of SEQ ID NO:43, and the LCDR3 comprises the amino acid sequence of SEQ ID NO: 44.

In some specific embodiments, in the ILT3 binding region provided herein, the HCDR1 comprises the amino acid sequence of SEQ ID NO:48, the HCDR2 comprises the amino acid sequence of SEQ ID NO:40, the HCDR3 comprises the amino acid sequence of SEQ ID NO:41, the LCDR1 comprises the amino acid sequence of SEQ ID NO:42, the LCDR2 comprises the amino acid sequence of SEQ ID NO:43, and the LCDR3 comprises the amino acid sequence of SEQ ID NO: 44.

In some specific embodiments, in the ILT3 binding region provided herein, the HCDR1 comprises the amino acid sequence of SEQ ID NO:49, the HCDR2 comprises the amino acid sequence of SEQ ID NO:50, the HCDR3 comprises the amino acid sequence of SEQ ID NO:51, the LCDR1 comprises the amino acid sequence of SEQ ID NO:52, the LCDR2 comprises the amino acid sequence of SEQ ID NO:53, and the LCDR3 comprises the amino acid sequence of SEQ ID NO: 54.

In other embodiments, the ILT3 binding region comprises a HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any one of SEQ ID NOs: 57, 63, 66 and 67; (ii) a HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any one of SEQ ID NOs: 58, 64, 65 and 68, (iii) a HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 59 or 69; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 60 or 70; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 61 or 71; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs: 62 or 72. In some embodiments, the ILT3 binding region is humanized. In some embodiments, the ILT3 binding region comprises an acceptor human framework, e.g., a human immunoglobulin framework or a human consensus framework.

In some specific embodiments, the ILT3 binding region provided herein comprises one or more CDRs in Table 4.

In some embodiments, the ILT3 binding region provided herein comprises a HCDR1 comprising the amino acid sequence of any one of SEQ ID NOs: 57, 63, 66 and 67; (ii) a HCDR2 comprising the amino acid sequence of any one of SEQ ID NOs: 58, 64, 65 and 68, (iii) a HCDR3 comprising the amino acid sequence of SEQ ID NOs: 59 or 69; (iv) a LCDR1 comprising the amino acid sequence of SEQ ID NOs: 60 or 70; (v) a LCDR2 comprising the amino acid sequence of SEQ ID NOs: 61 or 71; and/or (vi) a LCDR3 comprising the amino acid sequence of SEQ ID NOs: 62 or 72.

In some specific embodiments, in the ILT3 binding region provided herein, the HCDR1 comprises the amino acid sequence of SEQ ID NO:57, the HCDR2 comprises the amino acid sequence of SEQ ID NO:58, the HCDR3 comprises the amino acid sequence of SEQ ID NO:59, the LCDR1 comprises the amino acid sequence of SEQ ID NO:60, the LCDR2 comprises the amino acid sequence of SEQ ID NO:61, and the LCDR3 comprises the amino acid sequence of SEQ ID NO: 62.

In some specific embodiments, in the ILT3 binding region provided herein, the HCDR1 comprises the amino acid sequence of SEQ ID NO:63, the HCDR2 comprises the amino acid sequence of SEQ ID NO:64, the HCDR3 comprises the amino acid sequence of SEQ ID NO:59, the LCDR1 comprises the amino acid sequence of SEQ ID NO:60, the LCDR2 comprises the amino acid sequence of SEQ ID NO:61, and the LCDR3 comprises the amino acid sequence of SEQ ID NO: 62.

In some specific embodiments, in the ILT3 binding region provided herein, the HCDR1 comprises the amino acid sequence of SEQ ID NO:57, the HCDR2 comprises the amino acid sequence of SEQ ID NO:65, the HCDR3 comprises the amino acid sequence of SEQ ID NO:59, the LCDR1 comprises the amino acid sequence of SEQ ID NO:60, the LCDR2 comprises the amino acid sequence of SEQ ID NO:61, and the LCDR3 comprises the amino acid sequence of SEQ ID NO: 62.

In some specific embodiments, in the ILT3 binding region provided herein, the HCDR1 comprises the amino acid sequence of SEQ ID NO:66, the HCDR2 comprises the amino acid sequence of SEQ ID NO:58, the HCDR3 comprises the amino acid sequence of SEQ ID NO:59, the LCDR1 comprises the amino acid sequence of SEQ ID NO:60, the LCDR2 comprises the amino acid sequence of SEQ ID NO:61, and the LCDR3 comprises the amino acid sequence of SEQ ID NO: 62.

In some specific embodiments, in the ILT3 binding region provided herein, the HCDR1 comprises the amino acid sequence of SEQ ID NO:67, the HCDR2 comprises the amino acid sequence of SEQ ID NO:68, the HCDR3 comprises the amino acid sequence of SEQ ID NO:69, the LCDR1 comprises the amino acid sequence of SEQ ID NO:70, the LCDR2 comprises the amino acid sequence of SEQ ID NO:71, and the LCDR3 comprises the amino acid sequence of SEQ ID NO: 72.

In other embodiments, the ILT3 binding region comprises a HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any one of SEQ ID NOs: 75, 81, 84 and 85; (ii) a HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any one of SEQ ID NOs: 76, 82, 83 and 86, (iii) a HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 77 or 87; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 78 or 88; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 79 or 89; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs: 80 or 90. In some embodiments, the ILT3 binding region is humanized. In some embodiments, the ILT3 binding region comprises an acceptor human framework, e.g., a human immunoglobulin framework or a human consensus framework.

In some specific embodiments, the ILT3 binding region provided herein comprises one or more CDRs in Table 5.

In some embodiments, the ILT3 binding region provided herein comprises a HCDR1 comprising the amino acid sequence of any one of SEQ ID NOs: 75, 81, 84 and 85; (ii) a HCDR2 comprising the amino acid sequence of any one of SEQ ID NOs: 76, 82, 83 and 86, (iii) a HCDR3 comprising the amino acid sequence of SEQ ID NOs: 77 or 87; (iv) a LCDR1 comprising the amino acid sequence of SEQ ID NOs: 78 or 88; (v) a LCDR2 comprising the amino acid sequence of SEQ ID NOs: 79 or 89; and/or (vi) a LCDR3 comprising the amino acid sequence of SEQ ID NOs: 80 or 90.

In some specific embodiments, in the ILT3 binding region provided herein, the HCDR1 comprises the amino acid sequence of SEQ ID NO:75, the HCDR2 comprises the amino acid sequence of SEQ ID NO:76, the HCDR3 comprises the amino acid sequence of SEQ ID NO:77, the LCDR1 comprises the amino acid sequence of SEQ ID NO:78, the LCDR2 comprises the amino acid sequence of SEQ ID NO:79, and the LCDR3 comprises the amino acid sequence of SEQ ID NO: 80.

In some specific embodiments, in the ILT3 binding region provided herein, the HCDR1 comprises the amino acid sequence of SEQ ID NO:81, the HCDR2 comprises the amino acid sequence of SEQ ID NO:82, the HCDR3 comprises the amino acid sequence of SEQ ID NO:77, the LCDR1 comprises the amino acid sequence of SEQ ID NO:78, the LCDR2 comprises the amino acid sequence of SEQ ID NO:79, and the LCDR3 comprises the amino acid sequence of SEQ ID NO: 80.

In some specific embodiments, in the ILT3 binding region provided herein, the HCDR1 comprises the amino acid sequence of SEQ ID NO:75, the HCDR2 comprises the amino acid sequence of SEQ ID NO:83, the HCDR3 comprises the amino acid sequence of SEQ ID NO:77, the LCDR1 comprises the amino acid sequence of SEQ ID NO:78, the LCDR2 comprises the amino acid sequence of SEQ ID NO:79, and the LCDR3 comprises the amino acid sequence of SEQ ID NO: 80.

In some specific embodiments, in the ILT3 binding region provided herein, the HCDR1 comprises the amino acid sequence of SEQ ID NO:84, the HCDR2 comprises the amino acid sequence of SEQ ID NO:76, the HCDR3 comprises the amino acid sequence of SEQ ID NO:77, the LCDR1 comprises the amino acid sequence of SEQ ID NO:78, the LCDR2 comprises the amino acid sequence of SEQ ID NO:79, and the LCDR3 comprises the amino acid sequence of SEQ ID NO: 80.

In some specific embodiments, in the ILT3 binding region provided herein, the HCDR1 comprises the amino acid sequence of SEQ ID NO:85, the HCDR2 comprises the amino acid sequence of SEQ ID NO:86, the HCDR3 comprises the amino acid sequence of SEQ ID NO:87, the LCDR1 comprises the amino acid sequence of SEQ ID NO:88, the LCDR2 comprises the amino acid sequence of SEQ ID NO:89, and the LCDR3 comprises the amino acid sequence of SEQ ID NO: 90.

In other embodiments, the ILT3 binding region comprises a HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any one of SEQ ID NOs: 93, 99, 102 and 103; (ii) a HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any one of SEQ ID NOs: 94, 100, 101 and 104, (iii) a HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 95 or 105; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 96 or 106; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 97 or 107; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs: 98 or 108. In some embodiments, the ILT3 binding region is humanized. In some embodiments, the ILT3 binding region comprises an acceptor human framework, e.g., a human immunoglobulin framework or a human consensus framework.

In some specific embodiments, the ILT3 binding region provided herein comprises one or more CDRs in Table 6.

In some embodiments, the ILT3 binding region provided herein comprises a HCDR1 comprising the amino acid sequence of any one of SEQ ID NOs: 93, 99, 102 and 103; (ii) a HCDR2 comprising the amino acid sequence of any one of SEQ ID NOs: 94, 100, 101 and 104, (iii) a HCDR3 comprising the amino acid sequence of SEQ ID NOs: 95 or 105; (iv) a LCDR1 comprising the amino acid sequence of SEQ ID NOs: 96 or 106; (v) a LCDR2 comprising the amino acid sequence of SEQ ID NOs: 97 or 107; and/or (vi) a LCDR3 comprising the amino acid sequence of SEQ ID NOs: 98 or 108.

In some specific embodiments, in the ILT3 binding region provided herein, the HCDR1 comprises the amino acid sequence of SEQ ID NO:93, the HCDR2 comprises the amino acid sequence of SEQ ID NO:94, the HCDR3 comprises the amino acid sequence of SEQ ID NO:95, the LCDR1 comprises the amino acid sequence of SEQ ID NO:96, the LCDR2 comprises the amino acid sequence of SEQ ID NO:97, and the LCDR3 comprises the amino acid sequence of SEQ ID NO: 98.

In some specific embodiments, in the ILT3 binding region provided herein, the HCDR1 comprises the amino acid sequence of SEQ ID NO:99, the HCDR2 comprises the amino acid sequence of SEQ ID NO:100, the HCDR3 comprises the amino acid sequence of SEQ ID NO:95, the LCDR1 comprises the amino acid sequence of SEQ ID NO:96, the LCDR2 comprises the amino acid sequence of SEQ ID NO:97, and the LCDR3 comprises the amino acid sequence of SEQ ID NO: 98.

In some specific embodiments, in the ILT3 binding region provided herein, the HCDR1 comprises the amino acid sequence of SEQ ID NO:93, the HCDR2 comprises the amino acid sequence of SEQ ID NO:101, the HCDR3 comprises the amino acid sequence of SEQ ID NO:95, the LCDR1 comprises the amino acid sequence of SEQ ID NO:96, the LCDR2 comprises the amino acid sequence of SEQ ID NO:97, and the LCDR3 comprises the amino acid sequence of SEQ ID NO: 98.

In some specific embodiments, in the ILT3 binding region provided herein, the HCDR1 comprises the amino acid sequence of SEQ ID NO:102, the HCDR2 comprises the amino acid sequence of SEQ ID NO:94, the HCDR3 comprises the amino acid sequence of SEQ ID NO:95, the LCDR1 comprises the amino acid sequence of SEQ ID NO:96, the LCDR2 comprises the amino acid sequence of SEQ ID NO:97, and the LCDR3 comprises the amino acid sequence of SEQ ID NO: 98.

In some specific embodiments, in the ILT3 binding region provided herein, the HCDR1 comprises the amino acid sequence of SEQ ID NO:103, the HCDR2 comprises the amino acid sequence of SEQ ID NO:104, the HCDR3 comprises the amino acid sequence of SEQ ID NO: 105, the LCDR1 comprises the amino acid sequence of SEQ ID NO:106, the LCDR2 comprises the amino acid sequence of SEQ ID NO:107, and the LCDR3 comprises the amino acid sequence of SEQ ID NO:108.

In other embodiments, the ILT3 binding region comprises a HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any one of SEQ ID NOs: 111, 117, 120 and 121; (ii) a HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any one of SEQ ID NOs: 112, 118, 119 and 122, (iii) a HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 113 or 123; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 114 or 124; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 115 or 125; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs: 116 or 126. In some embodiments, the ILT3 binding region is humanized. In some embodiments, the ILT3 binding region comprises an acceptor human framework, e.g., a human immunoglobulin framework or a human consensus framework.

In some specific embodiments, the ILT3 binding region provided herein comprises one or more CDRs in Table 7.

In some embodiments, the ILT3 binding region provided herein comprises a HCDR1 comprising the amino acid sequence of any one of SEQ ID NOs: 111, 117, 120 and 121; (ii) a HCDR2 comprising the amino acid sequence of any one of SEQ ID NOs: 112, 118, 119 and 122, (iii) a HCDR3 comprising the amino acid sequence of SEQ ID NOs: 113 or 123; (iv) a LCDR1 comprising the amino acid sequence of SEQ ID NOs: 114 or 124; (v) a LCDR2 comprising the amino acid sequence of SEQ ID NOs: 115 or 125; and/or (vi) a LCDR3 comprising the amino acid sequence of SEQ ID NOs: 116 or 126.

In some specific embodiments, in the ILT3 binding region provided herein, the HCDR1 comprises the amino acid sequence of SEQ ID NO:111, the HCDR2 comprises the amino acid sequence of SEQ ID NO:112, the HCDR3 comprises the amino acid sequence of SEQ ID NO:113, the LCDR1 comprises the amino acid sequence of SEQ ID NO:114, the LCDR2 comprises the amino acid sequence of SEQ ID NO:115, and the LCDR3 comprises the amino acid sequence of SEQ ID NO:116.

In some specific embodiments, in the ILT3 binding region provided herein, the HCDR1 comprises the amino acid sequence of SEQ ID NO:117, the HCDR2 comprises the amino acid sequence of SEQ ID NO: 118, the HCDR3 comprises the amino acid sequence of SEQ ID NO:113, the LCDR1 comprises the amino acid sequence of SEQ ID NO:114, the LCDR2 comprises the amino acid sequence of SEQ ID NO:115, and the LCDR3 comprises the amino acid sequence of SEQ ID NO:116.

In some specific embodiments, in the ILT3 binding region provided herein, the HCDR1 comprises the amino acid sequence of SEQ ID NO:111, the HCDR2 comprises the amino acid sequence of SEQ ID NO:119, the HCDR3 comprises the amino acid sequence of SEQ ID NO: 113, the LCDR1 comprises the amino acid sequence of SEQ ID NO:114, the LCDR2 comprises the amino acid sequence of SEQ ID NO:115, and the LCDR3 comprises the amino acid sequence of SEQ ID NO:116.

In some specific embodiments, in the ILT3 binding region provided herein, the HCDR1 comprises the amino acid sequence of SEQ ID NO:120, the HCDR2 comprises the amino acid sequence of SEQ ID NO:112, the HCDR3 comprises the amino acid sequence of SEQ ID NO: 113, the LCDR1 comprises the amino acid sequence of SEQ ID NO:114, the LCDR2 comprises the amino acid sequence of SEQ ID NO:115, and the LCDR3 comprises the amino acid sequence of SEQ ID NO:116.

In some specific embodiments, in the ILT3 binding region provided herein, the HCDR1 comprises the amino acid sequence of SEQ ID NO:121, the HCDR2 comprises the amino acid sequence of SEQ ID NO: 122, the HCDR3 comprises the amino acid sequence of SEQ ID NO:123, the LCDR1 comprises the amino acid sequence of SEQ ID NO:124, the LCDR2 comprises the amino acid sequence of SEQ ID NO:125, and the LCDR3 comprises the amino acid sequence of SEQ ID NO:126.

In other embodiments, the ILT3 binding region comprises a HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any one of SEQ ID NOs: 129, 135, 138 and 139; (ii) a HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any one of SEQ ID NOs: 130, 136, 137 and 140, (iii) a HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 131 or 141; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 132 or 142; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 133 or 143; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs: 134 or 144. In some embodiments, the ILT3 binding region is humanized. In some embodiments, the ILT3 binding region comprises an acceptor human framework, e.g., a human immunoglobulin framework or a human consensus framework.

In some specific embodiments, the ILT3 binding region provided herein comprises one or more CDRs in Table 8.

In some embodiments, the ILT3 binding region provided herein comprises a HCDR1 comprising the amino acid sequence of any one of SEQ ID NOs: 129, 135, 138 and 139; (ii) a HCDR2 comprising the amino acid sequence of any one of SEQ ID NOs: 130, 136, 137 and 140, (iii) a HCDR3 comprising the amino acid sequence of SEQ ID NOs: 131 or 141; (iv) a LCDR1 comprising the amino acid sequence of SEQ ID NOs: 132 or 142; (v) a LCDR2 comprising the amino acid sequence of SEQ ID NOs: 133 or 143; and/or (vi) a LCDR3 comprising the amino acid sequence of SEQ ID NOs: 134 or 144.

In some specific embodiments, in the ILT3 binding region provided herein, the HCDR1 comprises the amino acid sequence of SEQ ID NO:129, the HCDR2 comprises the amino acid sequence of SEQ ID NO:130, the HCDR3 comprises the amino acid sequence of SEQ ID NO: 131, the LCDR1 comprises the amino acid sequence of SEQ ID NO: 132, the LCDR2 comprises the amino acid sequence of SEQ ID NO:133, and the LCDR3 comprises the amino acid sequence of SEQ ID NO:134.

In some specific embodiments, in the ILT3 binding region provided herein, the HCDR1 comprises the amino acid sequence of SEQ ID NO: 135, the HCDR2 comprises the amino acid sequence of SEQ ID NO: 136, the HCDR3 comprises the amino acid sequence of SEQ ID NO: 131, the LCDR1 comprises the amino acid sequence of SEQ ID NO: 132, the LCDR2 comprises the amino acid sequence of SEQ ID NO:133, and the LCDR3 comprises the amino acid sequence of SEQ ID NO: 134.

In some specific embodiments, in the ILT3 binding region provided herein, the HCDR1 comprises the amino acid sequence of SEQ ID NO:129, the HCDR2 comprises the amino acid sequence of SEQ ID NO: 137, the HCDR3 comprises the amino acid sequence of SEQ ID NO: 131, the LCDR1 comprises the amino acid sequence of SEQ ID NO:132, the LCDR2 comprises the amino acid sequence of SEQ ID NO:133, and the LCDR3 comprises the amino acid sequence of SEQ ID NO:134.

In some specific embodiments, in the ILT3 binding region provided herein, the HCDR1 comprises the amino acid sequence of SEQ ID NO:138, the HCDR2 comprises the amino acid sequence of SEQ ID NO: 130, the HCDR3 comprises the amino acid sequence of SEQ ID NO: 131, the LCDR1 comprises the amino acid sequence of SEQ ID NO: 132, the LCDR2 comprises the amino acid sequence of SEQ ID NO:133, and the LCDR3 comprises the amino acid sequence of SEQ ID NO:134.

In some specific embodiments, in the ILT3 binding region provided herein, the HCDR1 comprises the amino acid sequence of SEQ ID NO:139, the HCDR2 comprises the amino acid sequence of SEQ ID NO: 140, the HCDR3 comprises the amino acid sequence of SEQ ID NO: 141, the LCDR1 comprises the amino acid sequence of SEQ ID NO:142, the LCDR2 comprises the amino acid sequence of SEQ ID NO:143, and the LCDR3 comprises the amino acid sequence of SEQ ID NO:144.

In some embodiments, the ILT3 binding region further comprises one or more framework regions of the amino acid sequences of SEQ ID NO:17, SEQ ID NO:18, SEQ ID NO:37, SEQ ID NO:38, SEQ ID NO:55, SEQ ID NO:56, SEQ ID NO:73, SEQ ID NO:74, SEQ ID NO:91, SEQ ID NO:92, SEQ ID NO:109, SEQ ID NO:110, SEQ ID NO: 127, SEQ ID NO: 128, SEQ ID NO: 145 and SEQ ID NO:146. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system.

In some embodiments, the ILT3 binding region provided herein comprises a VH domain comprising the amino acid sequence of SEQ ID NO:17, and a VL domain comprising the amino acid sequence of SEQ ID NO:18. In some embodiments, the ILT3 binding region provided herein comprises a VH domain comprising the amino acid sequence of SEQ ID NO:37, and a VL domain comprising the amino acid sequence of SEQ ID NO:38. In some embodiments, the ILT3 binding region provided herein comprises a VH domain comprising the amino acid sequence of SEQ ID NO: 55, and a VL domain comprising the amino acid sequence of SEQ ID NO:56. In some embodiments, the ILT3 binding region provided herein comprises a VH domain comprising the amino acid sequence of SEQ ID NO:73, and a VL domain comprising the amino acid sequence of SEQ ID NO:74. In some embodiments, the ILT3 binding region provided herein comprises a VH domain comprising the amino acid sequence of SEQ ID NO:91, and a VL domain comprising the amino acid sequence of SEQ ID NO:92. In some embodiments, the ILT3 binding region provided herein comprises a VH domain comprising the amino acid sequence of SEQ ID NO:109, and a VL domain comprising the amino acid sequence of SEQ ID NO:110. In some embodiments, the ILT3 binding region provided herein comprises a VH domain comprising the amino acid sequence of SEQ ID NO:127, and a VL domain comprising the amino acid sequence of SEQ ID NO:128. In some embodiments, the ILT3 binding region provided herein comprises a VH domain comprising the amino acid sequence of SEQ ID NO:145, and a VL domain comprising the amino acid sequence of SEQ ID NO: 146.

In certain embodiments, the ILT3 binding region provided herein comprises amino acid sequences with certain percent identity relative to any ILT3 binding region provided herein (such as in Table 1, Table 2, Table 3, Table 4, Table 5, Table 6, Table 7 and Table 8).

The determination of percent identity between two sequences (e.g., amino acid sequences or nucleic acid sequences) can be accomplished using a mathematical algorithm. A non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and XBLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In some embodiments, the ILT3 binding region provide herein contains substitutions (e.g., conservative substitutions), insertions, or deletions relative to the reference sequence, but the ILT3 binding region comprising that sequence retains the ability to bind to ILT3. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). Optionally, the ILT3 binding region provided herein includes post-translational modifications of a reference sequence.

In some embodiments, the ILT3 binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:17, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:18.

In some embodiments, the ILT3 binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:37, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:38.

In some embodiments, the ILT3 binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:55, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:56.

In some embodiments, the ILT3 binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:73, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:74.

In some embodiments, the ILT3 binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:91, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:92.

In some embodiments, the ILT3 binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:109, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:110.

In some embodiments, the ILT3 binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:127, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:128.

In some embodiments, the ILT3 binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:145, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:146.

In some embodiments, the ILT3 binding region provided herein binds to the same epitope as an ILT3 binding region comprising a VH domain comprising the amino acid sequence of SEQ ID NO:17, and a VL domain comprising the amino acid sequence of SEQ ID NO:18. In some embodiments, the ILT3 binding region provided herein binds to the same epitope as an ILT3 binding region comprising a VH domain comprising the amino acid sequence of SEQ ID NO:37, and a VL domain comprising the amino acid sequence of SEQ ID NO:38. In some embodiments, the ILT3 binding region provided herein binds to the same epitope as an ILT3 binding region comprising a VH domain comprising the amino acid sequence of SEQ ID NO:55, and a VL domain comprising the amino acid sequence of SEQ ID NO:56. In some embodiments, the ILT3 binding region provided herein binds to the same epitope as an ILT3 binding region comprising a VH domain comprising the amino acid sequence of SEQ ID NO:73, and a VL domain comprising the amino acid sequence of SEQ ID NO:74. In some embodiments, the ILT3 binding region provided herein binds to the same epitope as an ILT3 binding region comprising a VH domain comprising the amino acid sequence of SEQ ID NO:91, and a VL domain comprising the amino acid sequence of SEQ ID NO:92. In some embodiments, the ILT3 binding region provided herein binds to the same epitope as an ILT3 binding region comprising a VH domain comprising the amino acid sequence of SEQ ID NO:109, and a VL domain comprising the amino acid sequence of SEQ ID NO: 110. In some embodiments, the ILT3 binding region provided herein binds to the same epitope as an ILT3 binding region comprising a VH domain comprising the amino acid sequence of SEQ ID NO: 127, and a VL domain comprising the amino acid sequence of SEQ ID NO:128. In some embodiments, the ILT3 binding region provided herein binds to the same epitope as an ILT3 binding region comprising a VH domain comprising the amino acid sequence of SEQ ID NO:145, and a VL domain comprising the amino acid sequence of SEQ ID NO:146.

In some embodiments, the ILT3 binding region provided herein specifically binds to ILT3 (e.g., human ILT3) competitively with an ILT3 binding region comprising a VH domain comprising the amino acid sequence of SEQ ID NO:17, and a VL domain comprising the amino acid sequence of SEQ ID NO:18. In some embodiments, the ILT3 binding region provided herein specifically binds to ILT3 (e.g., human ILT3) competitively with an ILT3 binding region comprising a VH domain comprising the amino acid sequence of SEQ ID NO:17, and a VL domain comprising the amino acid sequence of SEQ ID NO:18. In some embodiments, the ILT3 binding region provided herein specifically binds to ILT3 (e.g., human ILT3) competitively with an ILT3 binding region comprising a VH domain comprising the amino acid sequence of SEQ ID NO:37, and a VL domain comprising the amino acid sequence of SEQ ID NO:38. In some embodiments, the ILT3 binding region provided herein specifically binds to ILT3 (e.g., human ILT3) competitively with an ILT3 binding region comprising a VH domain comprising the amino acid sequence of SEQ ID NO:55, and a VL domain comprising the amino acid sequence of SEQ ID NO: 56. In some embodiments, the ILT3 binding region provided herein specifically binds to ILT3 (e.g., human ILT3) competitively with an ILT3 binding region comprising a VH domain comprising the amino acid sequence of SEQ ID NO:73, and a VL domain comprising the amino acid sequence of SEQ ID NO:74. In some embodiments, the ILT3 binding region provided herein specifically binds to ILT3 (e.g., human ILT3) competitively with an ILT3 binding region comprising a VH domain comprising the amino acid sequence of SEQ ID NO:91, and a VL domain comprising the amino acid sequence of SEQ ID NO:92. In some embodiments, the ILT3 binding region provided herein specifically binds to ILT3 (e.g., human ILT3) competitively with an ILT3 binding region comprising a VH domain comprising the amino acid sequence of SEQ ID NO:109, and a VL domain comprising the amino acid sequence of SEQ ID NO:110. In some embodiments, the ILT3 binding region provided herein specifically binds to ILT3 (e.g., human ILT3) competitively with an ILT3 binding region comprising a VH domain comprising the amino acid sequence of SEQ ID NO: 145, and a VL domain comprising the amino acid sequence of SEQ ID NO:146.

TABLE 1
45G10 Binding Region Sequences

| | Exemplary | Chothia | AbM | Kabat | Contact |
|---|---|---|---|---|---|
| Heavy | GFTFSDYGMH | GFTFSDY | GFTFSDYGM | DYGMH | SDYGMH |
| Chain | (SEQ ID NO: 1) | (SEQ ID NO: 7) | H | (SEQ ID NO: 10) | (SEQ ID |
| variable | | | (SEQ ID NO: 1) | | NO: 11) |
| region | | | | | |
| CDR1 | | | | | |
| Heavy | YIFSGSSTIYYAD | FSGSST | YIFSGSSTIY | YIFSGSSTIYYAD | WVAYIFSG |
| Chain | TVKG | (SEQ ID NO: 8) | (SEQ ID NO: 9) | TVKG | SSTIY |
| variable | (SEQ ID NO: 2) | | | (SEQ ID NO: 2) | (SEQ ID |
| region | | | | | NO: 12) |
| CDR2 | | | | | |
| Heavy | ADGRGAMDY | ADGRGAMD | ADGRGAMD | ADGRGAMDY | ARADGRG |
| Chain | (SEQ ID NO: 3) | Y | Y | (SEQ ID NO: 3) | AMD |
| variable | | (SEQ ID NO: 3) | (SEQ ID NO: 3) | | (SEQ ID |
| region | | | | | NO: 13) |
| CDR3 | | | | | |
| Light | RASQDISKFLN | RASQDISKFL | RASQDISKFL | RASQDISKFLN | SKFLNWY |
| Chain | (SEQ ID NO: 4) | N | N | (SEQ ID NO: 4) | (SEQ ID |
| variable | | (SEQ ID NO: 4) | (SEQ ID NO: 4) | | NO: 14) |
| region | | | | | |
| CDR1 | | | | | |
| Light | YTSRLHS | YTSRLHS | YTSRLHS | YTSRLHS | LLIYYTSRL |
| Chain | (SEQ ID NO: 5) | (SEQ ID NO: 5) | (SEQ ID NO: 5) | (SEQ ID NO: 5) | H |
| variable | | | | | (SEQ ID |
| region | | | | | NO: 15) |
| CDR2 | | | | | |
| Light | QQGNTLPWT | QQGNTLPWT | QQGNTLPWT | QQGNTLPWT | QQGNTLPW |
| Chain | (SEQ ID NO: 6) | (SEQ ID NO: 6) | (SEQ ID NO: 6) | (SEQ ID NO: 6) | (SEQ ID |
| variable | | | | | NO: 16) |
| region | | | | | |
| CDR3 | | | | | |

VH (SEQ ID NO: 17):
EVQLVESGGGLVQPGGSLRLSCAASGFTFSDYGMHWVRQAPGKGLEWVAYIFSGSS
TIYYADTVKGRFTISRDNAKNSLYLQMNSLRAEDTAVYYCARADGRGAMDYWGQG
TLVTVSS
VL (SEQ ID NO: 18):
DIQMTQSPSSLSASVGDRVTITCRASQDISKFLNWYQQKPGKAPKLLIYYTSRLHSGV
PSRFSGSGSGTDFTFTISSLQPEDIATYFCQQGNTLPWTFGGGTKLEIK

TABLE 2
3A3 Binding Region Sequences

| | Exemplary | Chothia | AbM | Kabat | Contact |
|---|---|---|---|---|---|
| Heavy | GFSLTSYGVH | GFSLTSY | GFSLTSYGVH | SYGVH | TSYGVH |
| Chain | (SEQ ID NO: 21) | (SEQ ID NO: 27) | (SEQ ID NO: 21) | (SEQ ID NO: 30) | (SEQ ID |
| variable | | | | | NO: 31) |
| region | | | | | |
| CDR1 | | | | | |
| Heavy | VIWPGGTINYN | WPGGT | VIWPGGTIN | VIWPGGTINYNS | WLGVIWPG |
| Chain | SALMS | (SEQ ID NO: 28) | (SEQ ID | ALMS | GTIN |
| variable | (SEQ ID NO: 22) | | NO: 29) | (SEQ ID NO: 22) | (SEQ ID |
| region | | | | | NO: 32) |
| CDR2 | | | | | |
| Heavy | DKYDGGWFAY | DKYDGGWFA | DKYDGGWFA | DKYDGGWFAY | ASDKYDGG |
| Chain | (SEQ ID NO: 23) | Y (SEQ ID NO: 23) | Y | (SEQ ID NO: 23) | WFA |
| variable | | | (SEQ ID NO: 23) | | (SEQ ID |
| region | | | | | NO: 33) |
| CDR3 | | | | | |
| Light | KASQNVRTAV | KASQNVRTAV | KASQNVRTAV | KASQNVRTAVA | RTAVAWY |
| Chain | A | A | A | (SEQ ID NO: 24) | (SEQ ID |
| variable | (SEQ ID NO: 24) | (SEQ ID NO: 24) | (SEQ ID | | NO: 34) |
| region | | | NO: 24) | | |
| CDR1 | | | | | |
| Light | LASNRHT | LASNRHT | LASNRHT | LASNRHT | ALIYLASNR |
| Chain | (SEQ ID NO: 25) | (SEQ ID NO: 25) | (SEQ ID NO: 25) | (SEQ ID NO: 25) | H |
| variable | | | | | (SEQ ID |
| region | | | | | NO: 35) |
| CDR2 | | | | | |
| Light | LQHLNYPLT | LQHLNYPLT | LQHLNYPLT | LQHLNYPLT | LQHLNYPL |
| Chain | (SEQ ID NO: 26) | (SEQ ID NO: 26) | (SEQ ID NO: 26) | (SEQ ID NO: 26) | (SEQ ID |
| variable | | | | | NO: 36) |
| region | | | | | |
| CDR3 | | | | | |

3A3 Heavy chain variable region (SEQ ID NO: 37)
QVQLKESGPGLVAPSQSLSITCTVSGFSLTSYGVHWVRQPPGKGLEWLGVIWPGGTIN
YNSALMSRLSISKDNSKSQVFLKLNSLQTDDTAMYYCASDKYDGGWFAYWGQGTL
VTVSA
3A3 Light chain variable region (SEQ ID NO: 38)
DIVMTQSQKFMSTSVGDRVSITCKASQNVRTAVAWYQQKPGQSPEALIYLASNRHTG
VPDRFTGSGSGTDFSLSISNVQSEDLADYFCLQHLNYPLTFGSGTKLEIK

TABLE 3
5A7 Binding Region Sequences

| | Exemplary | Chothia | AbM | Kabat | Contact |
|---|---|---|---|---|---|
| Heavy | GFTFSSYGMS | GFTFSSY | GFTFSSYGMS | SYGMS | SSYGMS |
| Chain | (SEQ ID NO: 39) | (SEQ ID | (SEQ ID | (SEQ ID NO: 48) | (SEQ ID NO: 49) |
| variable | | NO: 45) | NO: 39) | | |
| region | | | | | |
| CDR1 | | | | | |
| Heavy | TISGGGSYTNY | SGGGSY | TISGGGSYTN | TISGGGSYTNYP | WVATISGGGS |
| Chain | PDSVKG | (SEQ ID | (SEQ ID NO: 47) | DSVKG | YTN |
| variable | (SEQ ID NO: 40) | NO: 46) | | (SEQ ID NO: 40) | (SEQ ID NO: 50) |
| region | | | | | |
| CDR2 | | | | | |
| Heavy | REWRMTLYAM | REWRMTLYA | REWRMTL YA | REWRMTLYAM | ARREWRMTL |
| Chain | DY | MDY (SEQ ID | MDY (SEQ ID | DY | YAMD |
| variable | (SEQ ID NO: 41) | NO: 41) | NO: 41) | (SEQ ID NO: 41) | (SEQ ID NO: 51) |
| region | | | | | |
| CDR3 | | | | | |
| Light | RASESVDSYGN | RASESVDSYG | RASESVDSYG | RASESVDSYGN | DSYGNSFMH |
| Chain | SFMH | NSFMH | NSFMH | SFMH | WY |
| variable | (SEQ ID NO: 42) | (SEQ ID | (SEQ ID | (SEQ ID NO: 42) | (SEQ ID NO: 52) |
| region | | NO: 42) | NO: 42) | | |
| CDR1 | | | | | |
| Light | LTSNLES | LTSNLES | LTSNLES | LTSNLES | LLIYLTSNLE |
| Chain | (SEQ ID NO: 43) | (SEQ ID | (SEQ ID | (SEQ ID NO: 43) | (SEQ ID NO: 53) |
| variable | | NO: 43) | NO: 43) | | |
| region | | | | | |
| CDR2 | | | | | |
| Light | QQNNEDPFT | QQNNEDPFT | QQNNEDPFT | QQNNEDPFT | QQNNEDPF |
| Chain | (SEQ ID NO: 44) | (SEQ ID | (SEQ ID | (SEQ ID NO: 44) | (SEQ ID NO: 54) |
| variable | | NO: 44) | NO: 44) | | |
| region | | | | | |
| CDR3 | | | | | |

5A7 Heavy chain variable region (SEQ ID NO: 55)
EVKLVESGGGLVKPGGSLKLSCAASGFTFSSYGMSWVRQTPEKRLEWVATISGGGS
YTNYPDSVKGRLTISRDNAKKNLYLEMSSLRSEDTALYYCARREWRMTLYAMDYW
GQGTSVTVSS
5A7 Light chain variable region (SEQ ID NO: 56)
NIVLTQSPASLAVSLGQRATISCRASESVDSYGNSFMHWYQQKPGQAPKLLIYLTSNL
ESGVPARFSGSGSRTDFTLTIDPVEADDAATYYCQQNNEDPFTFGSGTKLEIK

TABLE 4
12A12 Binding Region Sequences

| | Exemplary | Chothia | AbM | Kabat | Contact |
|---|---|---|---|---|---|
| Heavy | GYTFTDYNMD | GYTFTDY | GYTFTDYNM | DYNMD | TDYNMD |
| Chain | (SEQ ID NO: 57) | (SEQ ID | D | (SEQ ID NO: 66) | (SEQ ID NO: 67) |
| variable | | NO: 63) | (SEQ ID | | |
| region | | | NO: 57) | | |
| CDR1 | | | | | |
| Heavy | YIYPNNGGTGY | YPNNGG | YIYPNNGGTG | YIYPNNGGTGY | WIGYIYPNNG |
| Chain | NQKENS | (SEQ ID | (SEQ ID | NQKFNS | GTG |
| variable | (SEQ ID NO: 58) | NO: 64) | NO: 65) | (SEQ ID NO: 58) | (SEQ ID NO: 68) |
| region | | | | | |
| CDR2 | | | | | |
| Heavy | SPYYDYVGSY | SPYYDYVGS | SPYYDYVGS | SPYYDYVGSYA | ASSPYYDYVG |
| Chain | AMDY | YAMDY | YAMDY | MDY | SYAMD |
| variable | (SEQ ID NO: 59) | (SEQ ID | (SEQ ID | (SEQ ID NO: 59) | (SEQ ID NO: 69) |
| region | | NO: 59) | NO: 59) | | |
| CDR3 | | | | | |
| Light | TASSSVSSSYL | TASSSVSSSY | TASSSVSSSY | TASSSVSSSYLH | SSSYLHWY |
| Chain | H | LH | LH | (SEQ ID NO: 60) | (SEQ ID NO: 70) |
| variable | (SEQ ID NO: 60) | (SEQ ID | (SEQ ID | | |
| region | | NO: 60) | NO: 60) | | |
| CDR1 | | | | | |
| Light | STSNLAS | STSNLAS | STSNLAS | STSNLAS | LWIYSTSNLA |
| Chain | (SEQ ID NO: 61) | (SEQ ID | (SEQ ID | (SEQ ID NO: 61) | (SEQ ID NO: 71) |
| variable | | NO: 61) | NO: 61) | | |
| region | | | | | |
| CDR2 | | | | | |
| Light | HQYHRSPRT | HQYHRSPRT | HQYHRSPRT | HQYHRSPRT | HQYHRSPR |
| Chain | (SEQ ID NO: 62) | (SEQ ID | (SEQ ID | (SEQ ID NO: 62) | (SEQ ID NO: 72) |
| variable | | NO: 62) | NO: 62) | | |
| region | | | | | |
| CDR3 | | | | | |

12A12 Heavy chain variable region (SEQ ID NO: 73)
EVQLQQSGPELVKPGASVKISCKASGYTFTDYNMDWVKQSHGKSLEWIGYIYPNNG
GTGYNQKFNSKATLTVDKSSSTAYMELHSLTSEDSAVYYCASSPYYDYVGSYAMDY
WGQGTSVTVSS
12A12 Light chain variable region (SEQ ID NO: 74)
QIVLTQSPAIMSASLGERVTMTCTASSSVSSSYLHWYQQKPGSSPKLWIYSTSNLASG
VPARFSGSGSGTSYSLTISSMEAEDAATYYCHQYHRSPRTFGGGTKLEIK

TABLE 5
16C5 Binding Region Sequences

| | Exemplary | Chothia | AbM | Kabat | Contact |
|---|---|---|---|---|---|
| Heavy | GYTFTDYNMD | GYTFTDY | GYTFTDYNM | DYNMD | TDYNMD |
| Chain | (SEQ ID NO: 75) | (SEQ ID | D | (SEQ ID NO: 84) | (SEQ ID NO: 85) |
| variable | | NO: 81) | (SEQ ID | | |
| region | | | NO: 75) | | |
| CDR1 | | | | | |
| Heavy | YIYPSNGGTGY | YPSNGG | YIYPSNGGTG | NQKFKS | WIGYIYPSNGG |
| Chain | NQKFKS | (SEQ ID | (SEQ ID NO: 83) | (SEQ ID NO: 76) | TG |
| variable | (SEQ ID NO: 76) | NO: 82) | | YIYPSNGGTGY | (SEQ ID NO: 86) |
| region | | | | | |
| CDR2 | | | | | |
| Heavy | VPYYDYLYYY | VPYYDYLYY | VPYYDYLYY | VPYYDYLYYY | ARVPYYDYLY |
| Chain | AMDY | YAMDY | YAMDY (SEQ ID | AMDY | YYAMD |
| variable | (SEQ ID NO: 77) | (SEQ ID | NO: 77) | (SEQ ID NO: 77) | (SEQ ID NO: 87) |
| region | | NO: 77) | | | |
| CDR3 | | | | | |
| Light | RASSSVSFMH | RASSSVSFMH | RASSSVSFMH | RASSSVSFMH | SFMHWY |
| Chain | (SEQ ID NO: 78) | (SEQ ID | (SEQ ID | (SEQ ID NO: 78) | (SEQ ID NO: 88) |
| variable | | NO: 78) | NO: 78) | | |
| region | | | | | |
| CDR1 | | | | | |
| Light | ATSNLAS | ATSNLAS | ATSNLAS | ATSNLAS | PWIYATSNLA |
| Chain | (SEQ ID NO: 79) | (SEQ ID | (SEQ ID | (SEQ ID NO: 79) | (SEQ ID NO: 89) |
| variable | | NO: 79) | NO: 79) | | |
| region | | | | | |
| CDR2 | | | | | |
| Light | QQWSTNPYMY | QQWSTNPYM | QQWSTNPYM | QQWSTNPYMY | QQWSTNPYM |
| Chain | T | YT | YT | T | Y |
| variable | (SEQ ID NO: 80) | (SEQ ID | (SEQ ID | (SEQ ID NO: 80) | (SEQ ID NO: 90) |
| region | | NO: 80) | NO: 80) | | |
| CDR3 | | | | | |

16C5 Heavy chain variable region (SEQ ID NO: 91)
EVQLQQSGPELVKPGASVKISCKASGYTFTDYNMDWVKQSHGKSLEWIGYIYPSNG
GTGYNQKFKSKATLTVDKSSNTAYMELHSLTSEDSAVYYCARVPYYDYLYYYAM
DYWGQGTSVTVSS
16C5 Light chain variable region (SEQ ID NO: 92)
QIVLSQSPAILSASPGEKVTMACRASSSVSFMHWYQQKPGSSPQPWIYATSNLASGV
PARFSGSGSGTSYSLTISRVEAEDAATYYCQQWSTNPYMYTFGGGTKLEIK

TABLE 6
48A6 Binding Region Sequences

| | Exemplary | Chothia | AbM | Kabat | Contact |
|---|---|---|---|---|---|
| Heavy | GFTFSSYGMS | GFTFSSY | GFTFSSYGMS | SYGMS | SSYGMS |
| Chain | (SEQ ID NO: 93) | (SEQ ID | (SEQ ID | (SEQ ID | (SEQ ID |
| variable | | NO: 99) | NO: 93) | NO: 102) | NO: 103) |
| region | | | | | |
| CDR1 | | | | | |
| Heavy | TISSGGTYTFYP | SSGGTY | TISSGGTYTF | TISSGGTYTFYP | WVATISSGGTY |
| Chain | DSVKG | (SEQ ID | (SEQ ID | DSVKG | TF |
| variable | (SEQ ID NO: 94) | NO: 100) | NO: 101) | (SEQ ID NO: 94) | (SEQ ID |
| region | | | | | NO: 104) |
| CDR2 | | | | | |
| Heavy | RGWLLHYYAM | RGWLLHYY | RGWLLHYYA | RGWLLHYYA | ARRGWLLHYY |
| Chain | DY | AMDY (SEQ ID | MDY (SEQ ID | MDY | AMD |
| variable | (SEQ ID NO: 95) | NO: 95) | NO: 95) | (SEQ ID NO: 95) | (SEQ ID |
| region | | | | | NO: 105) |
| CDR3 | | | | | |
| Light | RPSESVDSFGNS | RPSESVDSFG | RPSESVDSFG | RPSESVDSFGN | DSFGNSFMHW |
| Chain | FMH | NSFMH (SEQ ID | NSFMH (SEQ | SFMH | F |
| variable | (SEQ ID NO: 96) | NO: 96) | ID NO: 96) | (SEQ ID NO: 96) | (SEQ ID |
| region | | | | | NO: 106) |
| CDR1 | | | | | |
| Light | LSSKLES | LSSKLES | LSSKLES | LSSKLES | LLIYLSSKLE |
| Chain | (SEQ ID NO: 97) | (SEQ ID | (SEQ ID | (SEQ ID NO: 97) | (SEQ ID |
| variable | | NO: 97) | NO: 97) | | NO: 107) |
| region | | | | | |
| CDR2 | | | | | |
| Light | QQHNEDPFT | QQHNEDPFT | QQHNEDPFT | QQHNEDPFT | QQHNEDPF |
| Chain | (SEQ ID NO: 98) | (SEQ ID | (SEQ ID | (SEQ ID NO: 98) | (SEQ ID |
| variable | | NO: 98) | NO: 98) | | NO: 108) |
| region | | | | | |
| CDR3 | | | | | |

48A6 Heavy chain variable region (SEQ ID NO: 109)
EVQLVESGGDLMKPGGSLKLSCAASGFTFSSYGMSWVRQTPDKRLEWVATISSGGT
YTFYPDSVKGRFTISRDNAKNTLYLQMSSLKSEDTAMYYCARRGWLLHYYAMDYW
GQGTSVTVSS
48A6 Light chain variable region (SEQ ID NO: 110)
NIVLTQSPASLAVSLGQRATISCRPSESVDSFGNSFMHWFQQKPGQPPKLLIYLSSKLE
SGVPARFSGSGSRTDFTLTIDPVEADDAATYYCQQHNEDPFTFGSGTKLEIK

TABLE 7
53F10 Binding Region Sequences

| | Exemplary | Chothia | AbM | Kabat | Contact |
|---|---|---|---|---|---|
| Heavy | GFTFSDYGMH | GFTFSDY | GFTFSDYGM | DYGMH | SDYGMH |
| Chain | (SEQ ID NO: 111) | (SEQ ID | H | (SEQ ID NO: 120) | (SEQ ID |
| variable | | NO: 117) | (SEQ ID | | NO: 121) |
| region | | | NO: 111) | | |
| CDR1 | | | | | |
| Heavy | YISTGIITVYYAD | STGIIT | YISTGIITVY | YISTGIITVYYAD | WVAYISTGIIT |
| Chain | TVKG | (SEQ ID | (SEQ ID | TVKG (SEQ ID | VY |
| variable | (SEQ ID NO: 112) | NO: 118) | NO: 119) | NO: 112) | (SEQ ID |
| region | | | | | NO: 122) |
| CDR2 | | | | | |
| Heavy | ADGRGAMDY | ADGRGAMD | ADGRGAMD | ADGRGAMDY | ARADGRGAM |
| Chain | (SEQ ID NO: 113) | Y | Y | (SEQ ID NO: 113) | D |
| variable | | (SEQ ID | (SEQ ID | | (SEQ ID |
| region | | NO: 113) | NO: 113) | | NO: 123) |
| CDR3 | | | | | |
| Light | RASQDISNFLN | RASQDISNFL | RASQDISNFL | RASQDISNFLN | SNFLNWY |
| Chain | (SEQ ID NO: 114) | N (SEQ ID | N (SEQ ID | (SEQ ID NO: 114) | (SEQ ID |
| variable | | NO: 114) | NO: 114) | | NO: 124) |
| region | | | | | |
| CDR1 | | | | | |
| Light | YTSRLHS | YTSRLHS | YTSRLHS | YTSRLHS | LLIYYTSRLH |
| Chain | (SEQ ID NO: 115) | (SEQ ID | (SEQ ID | (SEQ ID NO: 115) | (SEQ ID |
| variable | | NO: 115) | NO: 115) | | NO: 125) |
| region | | | | | |
| CDR2 | | | | | |
| Light | QQGNTLPWT | QQGNTLPWT | QQGNTLPWT | QQGNTLPWT | QQGNTLPW |
| Chain | (SEQ ID NO: 116) | (SEQ ID | (SEQ ID | (SEQ ID NO: 116) | (SEQ ID |
| variable | | NO: 116) | NO: 116) | | NO: 126) |
| region | | | | | |
| CDR3 | | | | | |

53F10 Heavy chain variable region (SEQ ID NO: 127)
EVQVVESGGGLVKPGGSLKLSCAASGFTFSDYGMHWVRQAPEKGLEWVAYISTGII
TVYYADTVKGRFTMSRDNAKNTLFLQMTSLRSEDTAIYYCARADGRGAMDYWGQ
GTSVIVSS
53F10 Light chain variable region (SEQ ID NO: 128)
DIQMTQTTSSLSASLGDRVTISCRASQDISNFLNWYQQKPDGTVTLLIYYTSRLHSGV
PSRFSGSGSGTDYSLTISNLEQEDFATYFCQQGNTLPWTFGGGTKLEIK

TABLE 8
Hz5A7.v5 Binding Region Sequences

| | Exemplary | Chothia | AbM | Kabat | Contact |
|---|---|---|---|---|---|
| Heavy | GFTFSSYGMS | GFTFSSY | GFTFSSYGMS | SYGMS | SSYGMS |
| Chain | (SEQ ID NO: 129) | (SEQ ID | (SEQ ID | (SEQ ID NO: 138) | (SEQ ID |
| variable | | NO: 135) | NO: 129) | | NO: 139) |
| region | | | | | |
| CDR1 | | | | | |
| Heavy | TISGGGSYTNY | SGGGSY | TISGGGSYTN | TISGGGSYTNYP | WVATISGGG |
| Chain | PDSVKG | (SEQ ID | (SEQ ID | DSVKG | SYTN |
| variable | (SEQ ID NO: 130) | NO: 136) | NO: 137) | (SEQ ID NO: 130) | (SEQ ID |
| region | | | | | NO: 140) |
| CDR2 | | | | | |
| Heavy | REWRYTLYAM | REWRYTLYA | REWRYTLYA | REWRYTLYAM | ARREWRYTL |
| Chain | DY | MDY (SEQ ID | MDY (SEQ ID | DY | YAMD |
| variable | (SEQ ID NO: 131) | NO: 131) | NO: 131) | (SEQ ID NO: 131) | (SEQ ID |
| region | | | | | NO: 141) |
| CDR3 | | | | | |
| Light | RASESVESYGS | RASESVESYG | RASESVESYG | RASESVESYGSS | ESYGSSFMH |
| Chain | SFMH | SSFMH (SEQ ID | SSFMH (SEQ ID | FMH | WY |
| variable | (SEQ ID NO: 132) | NO: 132) | NO: 132) | (SEQ ID NO: 132) | (SEQ ID |
| region | | | | | NO: 142) |
| CDR1 | | | | | |
| Light | LTSNLES | LTSNLES | LTSNLES | LTSNLES | LLIYLTSNLE |
| Chain | (SEQ ID NO: 133) | (SEQ ID | (SEQ ID | (SEQ ID NO: 133) | (SEQ ID |
| variable | | NO: 133) | NO: 133) | | NO: 143) |
| region | | | | | |
| CDR2 | | | | | |
| Light | QQNNEDPFT | QQNNEDPFT | QQNNEDPFT | QQNNEDPFT | QQNNEDPF |
| Chain | (SEQ ID NO: 134) | (SEQ ID | (SEQ ID | (SEQ ID NO: 134) | (SEQ ID |
| variable | | NO: 134) | NO: 134) | | NO: 144) |
| region | | | | | |
| CDR3 | | | | | |

Hz5A7.v5 Heavy chain variable region (SEQ ID NO: 145)
EVQLVESGGGLVQPGGSLRLSCAASGFTFSSYGMSWVRQAPGKGLEWVATISGGGS
YTNYPDSVKGRFTISRDNAKNSLYLQMNSLRAEDTAVYYCARREWRYTLYAMDYW
GQGTTVTVSS
Hz5A7.v5 Light chain variable region (SEQ ID NO: 146)
DIQLTQSPSFLSASVGDRVTITCRASESVESYGSSFMHWYQQKPGKAPKLLIYLTSNLE
SGVPSRFSGSGSGTEFTLTISSLQPEDFATYYCQQNNEDPFTFGQGTKLEIK

### 5.2 CD3 Binding Regions

The binding agents provided here comprise a region that binds CD3 (e.g., human CD3), and thus the present binding agents are CD3 binding agents.

Amino acid (aa) sequences for human CD3 (UniProtKB No. P07766) and Cynomolgus monkey (“cyno”) CD3 (e.g., isoforms X1 (NCBI Ref No. XP_015290838.2) and X2 (NCB1 Ref No. XP_015290839.2) are known. CD3 is a single pass type I transmembrane protein with a predicted molecular weight of approximately 23 kDa. CD3 has been observed to be expressed on T cells, among other tissues. CD3 is characterized by an extracellular domain comprising paired Ig fold domains, a transmembrane domain, and a long cytoplasmic domain containing 1 ITAM domain (see, e.g., Kuhns et al., 2006, Immunity, 24.2:133-139). As characterized within UniProtKB, human CD3 is a protein of 207 amino acids (aa)—the signal sequence is aa 1-22, the extracellular domain is aa 23-126, the transmembrane region is aa 127-152, and the cytoplasmic domain is aa 153-207. Within the extracellular domain, the Ig-like domain is aa 32-112. Within the cytoplasmic domain, ITAMs are aa 178-205.

The present disclosure provides agents (e.g., bispecific antibodies) that bind CD3. In some embodiments, the CD3 binding agent binds a human CD3 or a fragment thereof.

In some embodiments, the CD3 binding region in the present binding agent is an antibody or a binding domain derived from an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In some embodiments, the antibody is an IgG antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody. In some embodiments, the antibody comprises an IgG heavy chain. In some embodiments, the antibody comprises an IgG1 heavy chain. In some embodiments, the antibody comprises an IgG2 heavy chain. In some embodiments, the antibody comprises an IgG4 heavy chain. In some embodiments, the antibody comprises a kappa light chain. In some embodiments, the antibody comprises a kappa light chain constant region. In some embodiments, the antibody comprises a lambda light chain. In some embodiments, the antibody comprises a lambda light chain constant region. In some embodiments, the antibody is an antibody fragment comprising an antigen-binding site. In some embodiments, the antibody is an scFv. In some embodiments, the antibody is a disulfide-linked scFv. In some embodiments, the antibody is a disulfide-linked sc (Fv)₂. In some embodiments, the antibody is a Fab, Fab′, or a F (ab)₂ antibody. In some embodiments, the antibody is a single chain Fab (scFab). In some embodiments, the antibody is a diabody. In some embodiments, the antibody is a nanobody. In some embodiments, the antibody is a monospecific antibody. In some embodiments, the antibody is a bispecific antibody. In some embodiments, the antibody is a monovalent antibody. In some embodiments, the antibody is a multivalent antibody. In some embodiments, the antibody is a bivalent antibody. In some embodiments, the antibody is a tetravalent antibody.

In some embodiments, the CD3 binding region is derived from a monoclonal antibody. Monoclonal antibodies can be prepared by any method known to those of skill in the art. In some embodiments, monoclonal antibodies are prepared using hybridoma methods as described in the Section above. In some embodiments, monoclonal antibodies are modified using recombinant DNA technology as described in the Section above.

In some embodiments, the CD3 binding region is derived from a humanized antibody. Various methods for generating humanized antibodies are known in the art. In some embodiments, a humanized antibody comprises one or more amino acid residues that have been introduced into it from a source that is non-human. In some embodiments, humanization is performed by substituting one or more non-human CDR sequences for the corresponding CDR sequences of a human antibody. In some embodiments, the humanized antibodies are constructed by substituting all six CDRs of a non-human antibody (e.g., a mouse antibody) for the corresponding CDRs of a human antibody. Other methods for humanization include those described in the Section above.

In some embodiments, the CD3 binding region is derived from a human antibody. Human antibodies can be prepared using various techniques known in the art, including those described in the Section above.

In some embodiments, the CD3 binding region is an antibody fragment. For example, antibody fragments include, but are not limited to, Fab, Fab′, F(ab′)₂, Fv, single chain antibody molecules (e.g., scFv), disulfide-linked scFv (dsscFv), nanobodies, diabodies, tribodies, tetrabodies, minibodies, dual variable domain antibodies (DVD), single variable domain antibodies (e.g., camelid antibodies), and multispecific antibodies formed from antibody fragments. Antibody fragments can be made by various techniques, including but not limited to those described in the Section above.

In some specific embodiments, the CD3 binding region comprises an anti-CD3 scFv. In some specific embodiments, the CD3 binding region comprises one or more Fabs. In some specific embodiments, the CD3 binding region comprises a Fab. In other specific embodiments, the CD3 binding region comprises two Fabs.

In some embodiments, the CD3 binding region provided herein binds to CD3 (e.g., human CD3) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸ M to 10⁻¹³ M, e.g., from 10⁻⁹M to 10⁻¹³ M). In some embodiments, the CD3 binding region provided herein binds to CD3 (e.g., human CD3) with a dissociation constant of ≤0.1 nM. In some embodiments, the CD3 binding region provided herein binds to CD3 (e.g., human CD3) with a dissociation constant of ≤0.01 nM. In some embodiments, the CD3 binding region provided herein binds to CD3 (e.g., human CD3) with a dissociation constant of ≤2 nM. In some embodiments, the CD3 binding region provided herein binds to CD3 (e.g., human CD3) with a dissociation constant of ≤3 nM. In some embodiments, the CD3 binding region provided herein binds to CD3 (e.g., human CD3) with a dissociation constant of ≤4 nM. In some embodiments, the CD3 binding region provided herein binds to CD3 (e.g., human CD3) with a dissociation constant of ≤300 nM.

In some embodiments, the CD3 binding region provided herein binds to CD3 (e.g., human CD3) with a K_{D} of from about 1 nM to about 1 μM. In some embodiments, the CD3 binding region provided herein binds to CD3 (e.g., human CD3) with a K_{D} from about 1 nM to about 500 nM, from about 1 nM to about 250 nM, from about 1 nM to about 100 nM, from about 1 nM to about 50 nM, from about 1 nM to about 25 nM, from about 1 nM to about 20 nM, from about 1 nM to about 15 nM, from about 1 nM to about 10 nM, from about 1 nM to about 5 nM, from about 5 nM to about 1 μM, from about 5 nM to about 500 nM, from about 5 nM to about 250 nM, from about 5 nM to about 100 nM, from about 5 nM to about 50 nM, from about 5 nM to about 25 nM, from about 5 nM to about 20 nM, from about 5 nM to about 15 nM, from about 5 nM to about 10 nM, from about 10 nM to about 1 μM, from about 10 nM to about 500 nM, from about 10 nM to about 250 nM, from about 10 nM to about 100 nM, from about 10 nM to about 50 nM, from about 10 nM to about 25 nM, from about 10 nM to about 20 nM, from about 10 nM to about 15 nM, from about 15 nM to about 1 μM, from about 15 nM to about 500 nM, from about 15 nM to about 250 nM, from about 15 nM to about 100 nM, from about 15 nM to about 50 nM, from about 15 nM to about 25 nM, from about 15 nM to about 20 nM, from about 20 nM to about 1 μM, from about 20 nM to about 500 nM, from about 20 nM to about 250 nM, from about 20 nM to about 100 nM, from about 20 nM to about 50 nM, from about 20 nM to about 25 nM, from about 25 nM to about 1 μM, from about 25 nM to about 500 nM, from about 25 nM to about 250 nM, from about 25 nM to about 100 nM, from about 25 nM to about 50 nM, from about 50 nM to about 1 μM, from about 50 nM to about 500 nM, from about 50 nM to about 250 nM, from about 50 nM to about 100 nM, from about 100 nM to about 1 μM, from about 100 nM to about 500 nM, from about 100 nM to about 250 nM, from about 250 nM to about 1 μM, from about 250 nM to about 500 nM or from about 500 nM to about 1μ M.

In some embodiments, the CD3 binding region provided herein binds to CD3 (e.g., human CD3) with a K_{D} of from about 5 nM to about 15 nM. In some embodiments, the CD3 binding region provided herein binds to CD3 (e.g., human CD3) with a K_{D} of from 6 nM to 13 nM. In some embodiments, the CD3 binding region provided herein binds to CD3 (e.g., human CD3) with a K_{D} of 6 nM as measured by SRP. In some embodiments, the CD3 binding region provided herein binds to CD3 (e.g., human CD3) with a K_{D} of from 6 nM to 13 nM as measured by different methods of measuring binding affinity.

A variety of methods of measuring binding affinity are known in the art, any one of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kₒₙ” may also be determined with the same biolayer interferometry (BLI) or surface plasmon resonance (SPR) techniques described above using, for example, the Octet®Red96, the Biacore®TM-3000, or the Biacore®TM-8000 system.

Any anti-CD3 antibodies known in the art can be used for deriving the CD3 binding region disclosed herein, for example, the anti-CD3 antibodies disclosed in Kuhn & Weiner, Immunotheray, 2016 July; 8 (8): 889-906, International Patent Publication Nos. WO2016204966, WO2017053856, WO2015095392, WO2016116626, WO2018114748, WO2005118635, and WO2014047231, the content of each of which is incorporated by reference herein.

In some embodiments, the CD3 binding region is derived from the antibody described in International Patent Publication No. WO 2008/119567 and U.S. Pat. No. 10,066,016, the content of each of which is incorporated by reference herein. In some embodiments, the CD3 binding region is as described in the example section below (see Section 7). In some embodiments, the CD3 binding region is 2B2 or a derivative thereof. In some embodiments, the CD3 binding region is 1G4 or a derivative thereof.

In some embodiments, the CD3 binding region provided herein comprises one or more CDR sequences of the amino acid sequences set forth in SEQ ID NOs: 149, 150, 158 and 159. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CD3 binding region is humanized. In some embodiments, the CD3 binding region comprises an acceptor human framework, e.g., a human immunoglobulin framework or a human consensus framework.

In some embodiments, the CD3 binding region provided herein comprises a HCDR1, a HCDR2, and a HCDR3 of the amino acid sequence set forth in SEQ ID NO: 149. In some embodiments, the CD3 binding region provided herein comprises a LCDR1, a LCDR2, and a LCDR3 of the amino acid sequence set forth in SEQ ID NO:150. In some embodiments, the CD3 binding region provided herein comprises a HCDR1, a HCDR2, and a HCDR3 of the amino acid sequence set forth in SEQ ID NO:149, and a LCDR1, a LCDR2, and a LCDR3 of the amino acid sequence set forth in SEQ ID NO:150. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the CD3 binding region comprises a HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 152; (ii) a HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO:153, (iii) a HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO:154; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO:155; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO:156; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO:157. In some embodiments, the CD3 binding region is humanized. In some embodiments, the CD3 binding region comprises an acceptor human framework, e.g., a human immunoglobulin framework or a human consensus framework.

In some specific embodiments, in the CD3 binding region provided herein, the HCDR1 comprises the amino acid sequence of SEQ ID NO:152, the HCDR2 comprises the amino acid sequence of SEQ ID NO: 153, the HCDR3 comprises the amino acid sequence of SEQ ID NO: 154, the LCDR1 comprises the amino acid sequence of SEQ ID NO:155, the LCDR2 comprises the amino acid sequence of SEQ ID NO:156, and the LCDR3 comprises the amino acid sequence of SEQ ID NO:157.

In some embodiments, the CD3 binding region further comprises one or more framework regions of the amino acid sequences of SEQ ID NOs: 149 and 150. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system.

In some embodiments, the CD3 binding region provided herein comprises a VH domain comprising the amino acid sequence of SEQ ID NO:149, and a VL domain comprising the amino acid sequence of SEQ ID NO: 150.

In certain embodiments, the CD3 binding region provided herein comprises amino acid sequences with certain percent identity relative to any CD3 binding region provided herein. The determination of percent identity can be accomplished using mathematical algorithms described in the Section above.

In some embodiments, the CD3 binding region provide herein contains substitutions (e.g., conservative substitutions), insertions, or deletions relative to the reference sequence, but the CD3 binding region comprising that sequence retains the ability to bind to CD3. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). Optionally, the CD3 binding region provided herein includes post-translational modifications of a reference sequence.

In some embodiments, the CD3 binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:149, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:150. In some embodiments, the CD3 binding region provided herein comprises a VH domain having at least 95%, at least 96%, at least 97%, at least 98%, or at least 99% sequence identity to the amino acid sequence of SEQ ID NO:149, and a VL domain having at least 90%, at least 91%, at least 92%, at least 93%, at least 94%, at least 95%, at least 96%, at least 97%, at least 98%, or at least 99% sequence identity to the amino acid sequence of SEQ ID NO: 150. In some embodiments, the CD3 binding region provided herein comprises a VH domain having at least 95% sequence identity to the amino acid sequence of SEQ ID NO:149, and a VL domain having at least 90% sequence identity to the amino acid sequence of SEQ ID NO:150. In some embodiments, the CD3 binding region provided herein comprises a VH domain having between 95% and 96% sequence identity to the amino acid sequence of SEQ ID NO: 149, and a VL domain having between 90% and 91% sequence identity to the amino acid sequence of SEQ ID NO: 150.

In some embodiments, the CD3 binding region provided herein binds to the same epitope as a CD3 binding region comprising a VH domain comprising the amino acid sequence of SEQ ID NO:149, and a VL domain comprising the amino acid sequence of SEQ ID NO:150.

In some embodiments, the CD3 binding region provided herein specifically binds to CD3 (e.g., human CD3) competitively with a CD3 binding region comprising a VH domain comprising the amino acid sequence of SEQ ID NO:149, and a VL domain comprising the amino acid sequence of SEQ ID NO: 150.

In some specific embodiments, the CD3 binding region is an scFv. In some embodiments, the scFv comprises one or more amino acid substitutions, such as those stabilizing scFv. In a specific embodiment, the scFv stabilization mutation is G44C mutation. In another specific embodiment, the scFv stabilization mutation is G100C mutation. In yet another embodiment, the scFv comprises both G44C and G100C mutations. In some embodiments, the anti-CD3 scFv comprises the amino acid sequence of SEQ ID NO:151.

In some embodiments, the anti-CD3 scFv comprises an amino acid sequence that has at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:151. In some embodiments, the anti-CD3 scFv comprises an amino acid sequence that has at least 92%, at least 93%, at least 94%, at least 95%, at least 96%, at least 97%, at least 98%, or at least 99% sequence identity to the amino acid sequence of SEQ ID NO: 151. In some embodiments, the anti-CD3 scFv comprises an amino acid sequence that has at least 92% sequence identity to the amino acid sequence of SEQ ID NO:151. In some embodiments, the anti-CD3 scFv comprises an amino acid sequence that has between 92% and 93% sequence identity to the amino acid sequence of SEQ ID NO:151.

In some embodiments, the CD3 binding region provided herein comprises a HCDR1, a HCDR2, and a HCDR3 of the amino acid sequence set forth in SEQ ID NO:158. In some embodiments, the CD3 binding region provided herein comprises a LCDR1, a LCDR2, and a LCDR3 of the amino acid sequence set forth in SEQ ID NO:159. In some embodiments, the CD3 binding region provided herein comprises a HCDR1, a HCDR2, and a HCDR3 of the amino acid sequence set forth in SEQ ID NO: 158, and a LCDR1, a LCDR2, and a LCDR3 of the amino acid sequence set forth in SEQ ID NO:159. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the CD3 binding region comprises a HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 161; (ii) a HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO:162, (iii) a HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO:163; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO:164; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 165; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO:166. In some embodiments, the CD3 binding region is humanized. In some embodiments, the CD3 binding region comprises an acceptor human framework, e.g., a human immunoglobulin framework or a human consensus framework.

In some specific embodiments, in the CD3 binding region provided herein, the HCDR1 comprises the amino acid sequence of SEQ ID NO: 161, the HCDR2 comprises the amino acid sequence of SEQ ID NO: 162, the HCDR3 comprises the amino acid sequence of SEQ ID NO: 163, the LCDR1 comprises the amino acid sequence of SEQ ID NO: 164, the LCDR2 comprises the amino acid sequence of SEQ ID NO:165, and the LCDR3 comprises the amino acid sequence of SEQ ID NO:166.

In some embodiments, the CD3 binding region further comprises one or more framework regions of the amino acid sequences of SEQ ID NOs: 158 and 159. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system.

In some embodiments, the CD3 binding region provided herein comprises a VH domain comprising the amino acid sequence of SEQ ID NO: 158, and a VL domain comprising the amino acid sequence of SEQ ID NO: 159.

In some embodiments, the CD3 binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:158, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:159. In some embodiments, the CD3 binding region provided herein comprises a VH domain having at least 97%, at least 98%, or at least 99% sequence identity to the amino acid sequence of SEQ ID NO:158, and a VL domain having at least 98%, or at least 99% sequence identity to the amino acid sequence of SEQ ID NO:159. In some embodiments, the CD3 binding region provided herein comprises a VH domain having at least 97% sequence identity to the amino acid sequence of SEQ ID NO:158, and a VL domain having at least 98% sequence identity to the amino acid sequence of SEQ ID NO:159. In some embodiments, the CD3 binding region provided herein comprises a VH domain having between 97% and 98% sequence identity to the amino acid sequence of SEQ ID NO:158, and a VL domain having between 98% and 99% sequence identity to the amino acid sequence of SEQ ID NO: 159.

In some embodiments, the CD3 binding region provided herein binds to the same epitope as a CD3 binding region comprising a VH domain comprising the amino acid sequence of SEQ ID NO:158, and a VL domain comprising the amino acid sequence of SEQ ID NO: 159.

In some embodiments, the CD3 binding region provided herein specifically binds to CD3 (e.g., human CD3) competitively with a CD3 binding region comprising a VH domain comprising the amino acid sequence of SEQ ID NO:158, and a VL domain comprising the amino acid sequence of SEQ ID NO: 159.

In some specific embodiments, the CD3 binding region is an scFv. In some embodiments, the scFv comprises one or more amino acid substitutions, such as those stabilizing scFv. In some embodiments, the scFv comprises the amino acid sequence of SEQ ID NO: 160.

### 5.3 Multispecific Binding Agents to ILT3 and CD3

ILT3×CD3 binding agents provided herein comprises an ILT3 binding region and an CD3 binding region, each of which is described in more detail in Section 5.1 and Section 5.2 above.

In certain embodiments, the ILT3 binding region has a higher binding affinity for human ILT3 than the binding affinity of the CD3 binding region for human CD3. In certain embodiments, the binding affinity of the ILT3 binding region for human ILT3 is between about 10 folds and about 100 folds higher than the binding affinity of the CD3 binding region for human CD3. In certain embodiments, the binding affinity of the ILT3 binding region for human ILT3 is between about 10 folds and about 100 folds, between about 10 folds and about 90 folds, between about 10 folds and about 80 folds, between about 10 folds and about 70 folds, between about 10 folds and about 60 folds, between about 10 folds and about 50 folds, between about 10 folds and about 40 folds, between about 10 folds and about 30 folds, between about 10 folds and about 20 folds, between about 20 folds and about 100 folds, between about 20 folds and about 90 folds, between about 20 folds and about 80 folds, between about 20 folds and about 70 folds, between about 20 folds and about 60 folds, between about 20 folds and about 50 folds, between about 20 folds and about 40 folds, between about 20 folds and about 30 folds, between about 30 folds and about 100 folds, between about 30 folds and about 90 folds, between about 30 folds and about 80 folds, between about 30 folds and about 70 folds, between about 30 folds and about 60 folds, between about 30 folds and about 50 folds, between about 30 folds and about 40 folds, between about 40 folds and about 100 folds, between about 40 folds and about 90 folds, between about 40 folds and about 80 folds, between about 40 folds and about 70 folds, between about 40 folds and about 60 folds, between about 40 folds and about 50 folds, between about 50 folds and about 100 folds, between about 50 folds and about 90 folds, between about 50 folds and about 80 folds, between about 50 folds and about 70 folds, between about 50 folds and about 60 folds, between about 60 folds and about 100 folds, between about 60 folds and about 90 folds, between about 60 folds and about 80 folds, between about 60 folds and about 70 folds, between about 70 folds and about 100 folds, between about 70 folds and about 90 folds, between about 70 folds and about 80 folds, between about 80 folds and about 100 folds, between about 80 folds and about 90 folds, or between about 90 folds and about 100 folds higher than the binding affinity of the CD3 binding region for human CD3. Such binding affinity difference between the two binding regions enables the ILT3×CD3 binding agents to bind the ILT3 expressing target cells before engaging any T cells, and thus reduces off-target effect and increases safety profile of the ILT3×CD3 binding agents disclosed herein.

In some embodiments, the ILT3×CD3 binding agents are multispecific antibodies such as bispecific antibodies. Any technologies known in the art for constructing multispecific antibodies or any multispecific formats known in the art may be used in constructing the present multispecific antibodies provided herein. Non-limiting exemplary technologies and formats are described below.

Binding agents provided herein can comprise antibodies having a full length antibody structure. “Full length antibody” refers to an antibody having two full length antibody heavy chains and two full length antibody light chains. A full length antibody heavy chain (HC) consists of well-known heavy chain variable and constant domains VH, CH1, hinge, CH2, and CH3. A full length antibody light chain (LC) consists of well-known light chain variable and constant domains VL and CL. The full length antibody can be lacking the C-terminal lysine (K) in either one or both heavy chains. “Fab-arm” or “half molecule” refers to one heavy chain-light chain pair that specifically binds an antigen.

Full length bispecific antibodies can be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy chain disulfide bonds in the hinge regions of the parental monospecific antibodies are reduced. The resulting free cysteines of one of the parental monospecific antibodies form an inter heavy-chain disulfide bond with cysteine residues of a second parental monospecific antibody molecule and simultaneously CH3 domains of the parental antibodies release and reform by dissociation-association. The CH3 domains of the Fab arms can be engineered to favor heterodimerization over homodimerization. The resulting product is a bispecific antibody having two Fab arms or half molecules which each bind a distinct epitope, i.e. an epitope on ILT3 and an epitope on CD3.

“Homodimerization” refers to an interaction of two heavy chains having identical CH3 amino acid sequences. “Homodimer” refers to an antibody having two heavy chains with identical CH3 amino acid sequences. “Heterodimerization” refers to an interaction of two heavy chains having non-identical CH3 amino acid sequences. “Heterodimer” refers to an antibody having two heavy chains with non-identical CH3 amino acid sequences.

In some embodiments, the binding agents provided herein include designs such as the Triomab/Quadroma (Trion Pharma/Fresenius Biotech), Knob-in-Hole (Genentech), CrossMAbs (Roche) and the electrostatically-matched (Chugai, Amgen, NovoNordisk, Oncomed), the LUZ-Y (Genentech), the Strand Exchange Engineered Domain body (SEEDbody) (EMD Serono), the Biclonic (Merus) and the DuoBody (Genmab A/S).

In some embodiments, the ILT3×CD3 binding agent provided herein is in a knob-in-hole format. In some embodiments, the CD3 binding region (e.g., anti-CD3 scFv) side of the Fc region bears the hole and the ILT3 binding region (e.g., anti-ILT3 Fab) side of the Fc region bears the knob.

In some embodiments, the ILT3×CD3 binding agent provided herein is in a DuoBody format.

The Triomab quadroma technology can be used to generate full length bispecific antibodies provided herein. Triomab technology promotes Fab arm exchange between two parental chimeric antibodies, one parental mAb having IgG2a and the second parental mAb having rat IgG2b constant regions, yielding chimeric bispecific antibodies.

The “knob-in-hole” strategy (see, e.g., International Publication No. WO 2006/028936) can be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob.” Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

The CrossMAb technology can be used to generate full length bispecific antibodies provided herein. CrossMAbs, in addition to utilizing the “knob-in-hole” strategy to promoter Fab arm exchange, have in one of the half arms the CH1 and the CL domains exchanged to ensure correct light chain pairing of the resulting bispecific antibody (see e.g. U.S. Pat. No. 8,242,247).

Other cross-over strategies can be used to generate full length bispecific antibodies provided herein by exchanging variable or constant, or both domains between the heavy chain and the light chain or within the heavy chain in the bispecific antibodies, either in one or both arms. These exchanges include for example VH-CH1 with VL-CL, VH with VL, CH3 with CL and CH3 with CH1 as described in International Publication Nos. WO 2009/080254, WO 2009/080251, WO 2009/018386 and WO 2009/080252.

Other strategies such as promoting heavy chain heterodimerization using electrostatic interactions by substituting positively charged residues at one CH3 surface and negatively charged residues at a second CH3 surface can be used, as described in US Pat. Publ. No. US2010/0015133; US Pat. Publ. No. US2009/0182127; US Pat. Publ. No. US2010/028637; or US Pat. Publ. No. US2011/0123532, the content of each of which is incorporated by reference herein. In other strategies, heterodimerization can be promoted by the following substitutions (expressed as modified position in the first CH3 domain of the first heavy chain/modified position in the second CH3 domain of the second heavy chain): L351Y_F405AY407V/T394W, T366I_K392M_T394W/F405A_Y407V, T366L_K392M_T394W/F405A_Y407V, L351Y_Y407A/T366A_K409F, L351Y_Y407A/T366V K409F Y407A/T366A_K409F, or T350V_L351Y_F405A Y407V/T350V_T366L_K392L_T394W as described in U.S. Pat. Publ. No. US2012/0149876 or U.S. Pat. Publ. No. US2013/0195849, the content of each of which is incorporated by reference herein.

LUZ-Y technology can be utilized to generate bispecific antibodies provided herein. In this technology, a leucine zipper is added into the C terminus of the CH3 domains to drive the heterodimer assembly from parental mAbs that is removed post-purification as described in Wranik et al., (2012) J Biol Chem 287 (52): 42221-9.

SEEDbody technology can be utilized to generate bispecific antibodies provided herein. SEEDbodies have, in their constant domains, select IgG residues substituted with IgA residues to promote heterodimerization as described in U.S. Patent No. US20070287170, the content of which is incorporated by reference herein.

In addition to methods described above, binding agents provided herein can be generated in vitro in a cell-free environment by introducing asymmetrical mutations in the CH3 regions of two mono specific homodimeric antibodies and forming the bispecific heterodimeric antibody from two parent monospecific homodimeric antibodies in reducing conditions to allow disulfide bond isomerization according to methods described in PCT Pat. Publ. No. WO 2011/131746.

In some embodiments described herein, the ILT3×CD3 bispecific antibody comprises a first binding region binding ILT3 and a second binding region binding CD3 and further comprises at least one substitution in an antibody CH3 constant domain. Substitutions are typically made at the DNA level to a molecule such as the constant domain of the antibody using standard methods.

The antibodies provided herein can be engineered into various well-known antibody forms.

In some embodiments, the bispecific antibody is a diabody or a cross-body.

In some embodiments, the bispecific antibody includes IgG-like molecules with complementary CH3 domains that promote heterodimerization; recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule.

In some embodiments, recombinant IgG-like dual targeting molecules include Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer).

In some embodiments, IgG fusion molecules include Dual Variable Domain (DVD)-Ig (Abbott), IgG-like Bispecific (ImClone/Eli Lilly), Ts2Ab (MedImmune/AZ) and BsAb (Zymogenetics), HERCULES (Biogen Idec) and TvAb (Roche).

In some embodiments, Fc fusion molecules can include ScFv/Fc Fusions (Academic Institution), SCORPION (Emergent BioSolutions/Trubion, Zymogenetics/BMS), Dual Affinity Retargeting Technology (Fc-DART) (MacroGenics) and Dual (ScFv)₂-Fab (National Research Center for Antibody Medicine-China).

In some embodiments, Fab fusion bispecific antibodies include F (ab)₂ (Medarex/AMGEN), Dual-Action or Bis-Fab (Genentech), Dock-and-Lock (DNL) (ImmunoMedics), Bivalent Bispecific (Biotecnoland Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies. Various formats of bispecific antibodies have been described, for example in Chames and Baty (2009) Curr Opin Drug Disc Dev 12:276 and in Nunez-Prado et al., (2015) Drug Discovery Today 20 (5): 588-594.

Any of the VH and the VL domains identified herein (e.g., those that bind CD3) can be engineered into an scFv format. In some embodiments, the scFv format is in the VH-linker-VL orientation. In other embodiments, the scFv format is in the VL-linker-VH orientation. Any of the VH and the VL domains identified herein can also be used to generate sc (Fv)₂ structures. In some embodiments, the sc (Fv)₂ structure is VH-linker-VL-linker-VL-linker-VH. In some embodiments, the sc (Fv)₂ structure is VH-linker-VL-linker-VH-linker-VL. In some embodiments, the sc (Fv)₂ structure is VH-linker-VH-linker-VL-linker-VL. In some embodiments, the sc (Fv)₂ structure is VL-linker-VH-linker-VH-linker-VL. In some embodiments, the sc (Fv)₂ structure is VL-linker-VH-linker-VL-linker-VH. In some embodiments, the sc (Fv)₂ structure is VL-linker-VL-linker-VH-linker-VH.

In specific embodiments, the linker is a peptide linker. In some embodiments, the liker comprises a naturally occurring amino acid. Exemplary amino acids that can be included into the linker are Gly, Ser Pro, Thr, Glu, Lys, Arg, Ile, Leu, His and The. In some embodiments, the linker has a length that is adequate to link the VH and the VL or the heavy chain and light chain of a Fab in such a way that they form the correct conformation relative to one another so that they retain the desired activity, such as binding to the target (e.g., ILT3 or CD3).

In certain embodiments, the linker is about 5-50 amino acids long. In some embodiments, the linker is about 10-40 amino acids long. In some embodiments, the linker is about 10-35 amino acids long. In some embodiments, the linker is about 10-30 amino acids long. In some embodiments, the linker is about 10-25 amino acids long. In some embodiments, the linker is about 10-20 amino acids long. In some embodiments, the linker is about 15-20 amino acids long. In some embodiments, the linker is 6 amino acids long. In some embodiments, the linker is 7 amino acids long. In some embodiments, the linker is 8 amino acids long. In some embodiments, the linker is 9 amino acids long. In some embodiments, the linker is 10 amino acids long. In some embodiments, the linker is 11 amino acids long. In some embodiments, the linker is 12 amino acids long. In some embodiments, the linker is 13 amino acids long. In some embodiments, the linker is 14 amino acids long. In some embodiments, the linker is 15 amino acids long. In some embodiments, the linker is 16 amino acids long. In some embodiments, the linker is 17 amino acids long. In some embodiments, the linker is 18 amino acids long. In some embodiments, the linker is 19 amino acids long. In some embodiments, the linker is 20 amino acids long. In some embodiments, the linker is 21 amino acids long. In some embodiments, the linker is 22 amino acids long. In some embodiments, the linker is 23 amino acids long. In some embodiments, the linker is 24 amino acids long. In some embodiments, the linker is 25 amino acids long. In some embodiments, the linker is 26 amino acids long. In some embodiments, the linker is 27 amino acids long. In some embodiments, the linker is 28 amino acids long. In some embodiments, the linker is 29 amino acids long. In some embodiments, the linker is 30 amino acids long. In some embodiments, the linker is 31 amino acids long. In some embodiments, the linker is 32 amino acids long. In some embodiments, the linker is 33 amino acids long. In some embodiments, the linker is 34 amino acids long. In some embodiments, the linker is 35 amino acids long. In some embodiments, the linker is 36 amino acids long. In some embodiments, the linker is 37 amino acids long. In some embodiments, the linker is 38 amino acids long. In some embodiments, the linker is 39 amino acids long. In some embodiments, the linker is 40 amino acids long. Exemplary linkers that can be used are Gly rich linkers, Gly and Ser containing linkers, Gly and Ala containing linkers, Ala and Ser containing linkers, and other flexible linkers. Exemplary linkers include the sequence (G₄S) n, wherein n=1-10, e.g., 1-5 or 2-5, for examples 2, 3, 4, or 5.

Exemplary linkers that can be used include any one of the linkers described in, for example, International Patent Application No. WO 2019/060695, the content of which is incorporated by reference herein. In certain embodiments, antibodies, including provided herein comprise two linkers. In other embodiments antibodies provided herein comprise three linkers. In yet other embodiments, antibodies provided herein comprise four or more linkers. In certain embodiments, the antibody is an antigen binding fragment thereof.

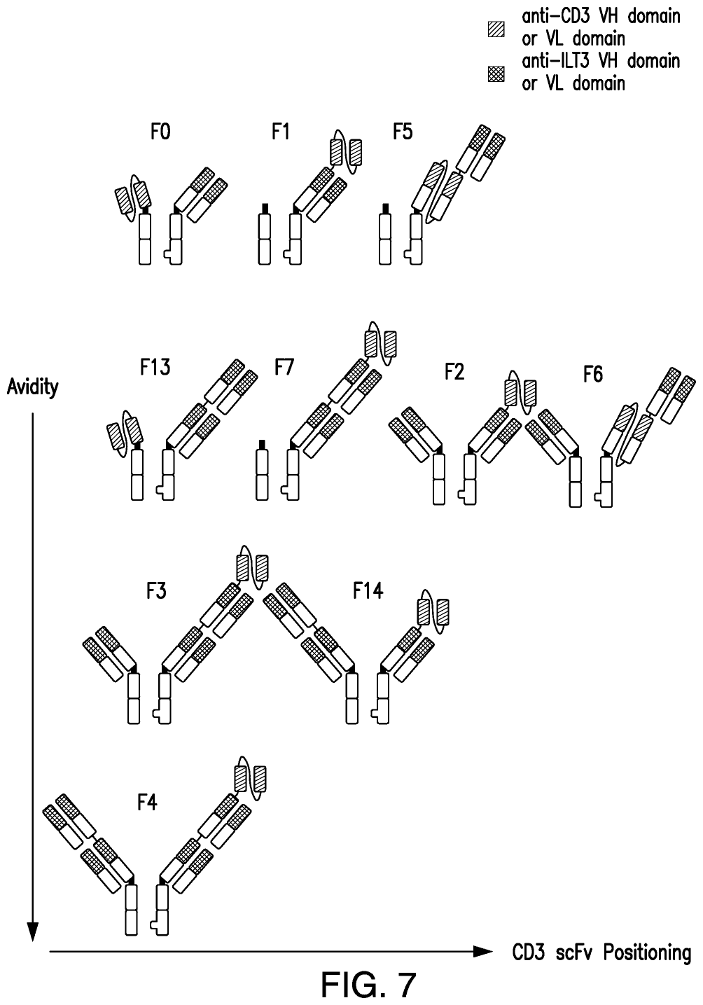

In some specific embodiments, the ILT3×CD3 binding agent provided herein is configured into any one of the formats disclosed in FIG. 7. In some specific embodiments, the ILT3×CD3 binding agent provided herein has a format of F0 as shown in FIG. 7. In some specific embodiments, the ILT3×CD3 binding agent provided herein has a format of F1 as shown in FIG. 7. In some specific embodiments, the ILT3×CD3 binding agent provided herein has a format of F5 as shown in FIG. 7. In some specific embodiments, the ILT3×CD3 binding agent provided herein has a format of F13 as shown in FIG. 7. In some specific embodiments, the ILT3×CD3 binding agent provided herein has a format of F7 as shown in FIG. 7. In some specific embodiments, the ILT3×CD3 binding agent provided herein has a format of F2 as shown in FIG. 7. In some specific embodiments, the ILT3×CD3 binding agent provided herein has a format of F6 as shown in FIG. 7. In some specific embodiments, the ILT3×CD3 binding agent provided herein has a format of F3 as shown in FIG. 7. In some specific embodiments, the ILT3×CD3 binding agent provided herein has a format of F14 as shown in FIG. 7. In some specific embodiments, the ILT3×CD3 binding agent provided herein has a format of F4 as shown in FIG. 7.

In some embodiments, the ILT3×CD3 binding agent provided herein comprises an scFv that binds CD3 and a Fab that binds ILT3, and the binding agent further comprises a Fc region. In certain embodiments, having the CD3 binding region as an scFv format improves the cytotoxicity and reduces the cytokine release of the ILT3×CD3 binding agent provided herein.

In some embodiments, the Fc region disclosed herein is altered to have reduced Fc-mediated effector functions, such as via reduced Fc receptor binding. In some embodiments, the Fc region is altered at one or more of the following amino acid positions to reduce Fc receptor binding: Leu 234 (L234), Leu235 (L235), Asp265 (D265), Asp270 (D270), Ser298 (S298), Asn297 (N297), Asn325 (N325) and Ala327 (A327). In certain embodiments, the Fc region comprises one or more of the following amino acid substitutions: Leu 234Ala (L234A), Leu235Ala (L235A), Asp265Asn (D265N), Asp270Asn (D270N), Ser298Asn (S298N), Asn297Ala (N297A), Asn325Glu (N325E) and Ala327Ser (A327S). In some embodiments, the Fc region is altered at both amino acid 234 and 235, e.g., Leu234Ala and Leu235Ala (L234A/L235A). Reference to amino acid substitutions in an Fc region is by EU numbering by Kabat. EU numbering is known and is according to the most recently updated IMGT Scientific Chart (IMGT®, the international ImMunoGeneTics information System®) and the EU index as reported in Kabat, E. A. et al. Sequences of Proteins of Immunological interest. 5th ed. US Department of Health and Human Services, NIH publication No. 91-3242 (1991).

In some embodiments, the ILT3×CD3 binding agent provided herein comprises: (i) a first polypeptide comprising an scFv that binds CD3 that is linked to one arm of a Fc region, (ii) a second polypeptide comprising the VH domain that binds ILT3 that is linked to the other arm of the Fc region, and (iii) a third polypeptide comprising the VL domain that binds ILT3, wherein the VH domain and the VL domain form a Fab that binds ILT3, and the first polypeptide and the second polypeptide form the Fc region. In some embodiments, the first polypeptide comprises a full or partial hinge domain. In some embodiments, the second polypeptide comprises a full or partial hinge domain. In some embodiments, the Fc region comprises one or more amino acid mutations that reduces or eliminate Fc effector functions. In some embodiments, the Fc region comprises L234A/L235A mutations. In some embodiments, the Fc region comprises one or more amino acid mutations that facilitate the dimerization of the two arms of the Fc region. In certain embodiments, the first polypeptide comprises one or more of T366S, L368A and Y407V mutations (e.g., all of T366S, L368A and Y407V mutations) at the domain (e.g., CH3 domain) that forms the Fc region, and the second polypeptide comprises T366W mutation at the domain (e.g., CH3 domain) that forms the Fc region.

In some embodiments, the ILT3×CD3 binding agent provided herein comprises: (i) a first polypeptide comprising an scFv that binds CD3 that is linked to one arm of a Fc region, (ii) a second polypeptide comprising the VH domain that binds ILT3 and a CH1 domain that is linked to the other arm of the Fc region, and (iii) a third polypeptide comprising the VL domain that binds ILT3 and a CL domain, wherein the VH domain, the CH1 domain, the CL domain, and the VL domain form a Fab that binds ILT3, and the first polypeptide and the second polypeptide form the Fc region. In some embodiments, the first polypeptide comprises a full or partial hinge domain. In some embodiments, the second polypeptide comprises a full or partial hinge domain. In some embodiments, the Fc region comprises one or more amino acid mutations that reduces or eliminate Fc effector functions. In some embodiments, the Fc region comprises L234A/L235A mutations. In some embodiments, the Fc region comprises one or more amino acid mutations that facilitate the dimerization of the two arms of the Fc region. In certain embodiments, the first polypeptide comprises one or more of T366S, L368A and Y407V mutations (e.g., all of T366S, L368A and Y407V mutations) at the domain (e.g., CH3 domain) that forms the Fc region, and the second polypeptide comprises T366W mutation at the domain (e.g., CH3 domain) that forms the Fc region.

In some embodiments, the ILT3×CD3 binding agent provided herein comprises: (i) a first polypeptide comprising an scFv that binds CD3, a CH2 domain, and a CH3 domain, (ii) a second polypeptide comprising the VH domain that binds ILT3, a CH2 domain, and a CH3 domain, and (iii) a third polypeptide comprising the VL domain that binds ILT3, wherein the VH domain and the VL domain form a Fab that binds ILT3, and the first polypeptide and the second polypeptide form a Fc region. In some embodiments, the first polypeptide comprises a full or partial hinge domain. In some embodiments, the second polypeptide comprises a full or partial hinge domain. In some embodiments, the Fc region comprises one or more amino acid mutations that reduces or eliminate Fc effector functions. In some embodiments, the Fc region comprises L234A/L235A mutations. In some embodiments, the Fc region comprises one or more amino acid mutations that facilitate the dimerization of the two arms of the Fc region. In certain embodiments, the CH3 domain of the first polypeptide comprises one or more of T366S, L368A and Y407V mutations (e.g., all of T366S, L368A and Y407V mutations), and the CH3 domain of the second polypeptide comprises T366W mutation.

In some embodiments, the ILT3×CD3 binding agent provided herein comprises: (i) a first polypeptide comprising an scFv that binds CD3, a CH2 domain, and a CH3 domain, (ii) a second polypeptide comprising the VH domain that binds ILT3, a CH1 domain, a CH2 domain, and a CH3 domain, and (iii) a third polypeptide comprising the VL domain that binds ILT3 and a CL domain, wherein the VH domain, the CH1 domain, the CL domain, and the VL domain form a Fab that binds ILT3, and the first CH2 domain, the second CH2 domain, the first CH3 domain, and the second CH3 domain form the Fc region. In some embodiments, the first polypeptide comprises a full or partial hinge domain. In some embodiments, the second polypeptide comprises a full or partial hinge domain. In some embodiments, the Fc region comprises one or more amino acid mutations that reduces or eliminate Fc effector functions. In some embodiments, the Fc region comprises L234A/L235A mutations. In some embodiments, the Fc region comprises one or more amino acid mutations that facilitate the dimerization of the two arms of the Fc region. In certain embodiments, the CH3 domain of the first polypeptide comprises one or more of T366S, L368A and Y407V mutations (e.g., all of T366S, L368A and Y407V mutations), and the CH3 domain of the second polypeptide comprises T366W mutation. In certain embodiments, the ILT3×CD3 binding agent has the configuration as depicted in F0 of FIG. 7.

In some specific embodiments, the ILT3×CD3 binding agent provided herein comprises a first polypeptide comprising the amino acid sequence of SEQ ID NO:147, a second polypeptide comprising the amino acid sequence of SEQ ID NO: 19, and a third polypeptide comprising the amino acid sequence of SEQ ID NO:20.

In other embodiments, the ILT3×CD3 binding agent provided herein comprises an scFv that binds CD3 and two Fabs each bind ILT3, and the binding agent further comprises a Fc region. In some embodiments, the two Fabs are identical and are linked to each other.

In some embodiments, the ILT3×CD3 binding agent provided herein comprises: (i) a first polypeptide comprising an scFv that binds CD3 that is linked to one arm of a Fc region, (ii) a second polypeptide comprising two identical VH domains in tandem each bind ILT3 that is linked to the other arm of the Fc region, (iii) a third polypeptide comprising a VL domain that binds ILT3, and (iv) a fourth polypeptide comprising a VL domain that binds ILT3, wherein the two VH domains and the two VL domains form two Fabs that bind ILT3, and the first polypeptide and the second polypeptide form the Fc region. In some embodiments, the first polypeptide comprises a full or partial hinge domain. In some embodiments, the second polypeptide comprises a full or partial hinge domain. In some embodiments, the Fc region comprises one or more amino acid mutations that reduces or eliminate Fc effector functions. In some embodiments, the Fc region comprises L234A/L235A mutations. In some embodiments, the Fc region comprises one or more amino acid mutations that facilitate the dimerization of the two arms of the Fc region. In certain embodiments, the first polypeptide comprises one or more of T366S, L368A and Y407V mutations (e.g., all of T366S, L368A and Y407V mutations) at the domain (e.g., CH3 domain) that forms the Fc region, and the second polypeptide comprises T366W mutation at the domain (e.g., CH3 domain) that forms the Fc region.

In some embodiments, the ILT3×CD3 binding agent provided herein comprises: (i) a first polypeptide comprising an scFv that binds CD3 that is linked to one arm of a Fc region, (ii) a second polypeptide comprising two identical VH domains each bind ILT3 and two identical CH1 domains, wherein one of the CH1 domains is linked to the other arm of the Fc region, (iii) a third polypeptide comprising a VL domain that binds ILT3 and a CL domain, and (iv) a fourth polypeptide comprising a VL domain that binds ILT3 and a CL domain, wherein the two VH domains, the two VL domains, the two CH1 domains, and the two CL domains form two Fabs that bind ILT3, and the first polypeptide and the second polypeptide form the Fc region. In some embodiments, the first polypeptide comprises a full or partial hinge domain. In some embodiments, the second polypeptide comprises a full or partial hinge domain. In some embodiments, the Fc region comprises one or more amino acid mutations that reduces or eliminate Fc effector functions. In some embodiments, the Fc region comprises L234A/L235A mutations. In some embodiments, the Fc region comprises one or more amino acid mutations that facilitate the dimerization of the two arms of the Fc region. In certain embodiments, the first polypeptide comprises one or more of T366S, L368A and Y407V mutations (e.g., all of T366S, L368A and Y407V mutations) at the domain (e.g., CH3 domain) that forms the Fc region, and the second polypeptide comprises T366W mutation at the domain (e.g., CH3 domain) that forms the Fc region.

In some embodiments, the ILT3×CD3 binding agent provided herein comprises: (i) a first polypeptide comprising an scFv that binds CD3, a CH2 domain, and a CH3 domain, (ii) a second polypeptide comprising a first VH domain that binds ILT3, a second VH domain that binds ILT3, a CH2 domain, and a CH3 domain, (iii) a third polypeptide comprising a first VL domain that binds ILT3, and (iv) a fourth polypeptide comprising a second VH domain that binds ILT3, wherein the first VH domain and the first VL domain form a first Fab that binds ILT3, the second VH domain and the second VL domain form a second Fab that binds ILT3, and the first polypeptide and the second polypeptide form a Fc region. In some embodiments, the first polypeptide comprises a full or partial hinge domain. In some embodiments, the second polypeptide comprises a full or partial hinge domain. In some embodiments, the Fc region comprises one or more amino acid mutations that reduces or eliminate Fc effector functions. In some embodiments, the Fc region comprises L234A/L235A mutations. In some embodiments, the Fc region comprises one or more amino acid mutations that facilitate the dimerization of the two arms of the Fc region. In certain embodiments, the first polypeptide comprises one or more of T366S, L368A and Y407V mutations (e.g., all of T366S, L368A and Y407V mutations) at the domain (e.g., CH3 domain) that forms the Fc region, and the second polypeptide comprises T366W mutation at the domain (e.g., CH3 domain) that forms the Fc region.

In some embodiments, the ILT3×CD3 binding agent provided herein comprises: (i) a first polypeptide comprising an scFv that binds CD3, a first CH2 domain, and a first CH3 domain, (ii) a second polypeptide comprising a first VH domain that binds ILT3, a first CH1 domain, a second VH domain that binds ILT3, a second CH1 domain, a second CH2 domain, and a second CH3 domain, (iii) a third polypeptide comprising a first VL domain that binds ILT3 and a first CL domain, and (iv) a fourth polypeptide comprising a second VL domain that binds ILT3 and a second CL domain, wherein the first VH domain, the first CH1 domain, the first VL domain, and the first CL domain form a first Fab that binds ILT3, the second VH domain, the second CH1 domain, the second VL domain, and the second CL domain form a second Fab that binds ILT3, and the first CH2 domain, the second CH2 domain, the first CH3 domain, and the second CH3 domain form the Fc region. In some embodiments, the first polypeptide comprises a full or partial hinge domain. In some embodiments, the second polypeptide comprises a full or partial hinge domain. In some embodiments, the Fc region comprises one or more amino acid mutations that reduces or eliminate Fc effector functions. In some embodiments, the Fc region comprises L234A/L235A mutations. In some embodiments, the Fc region comprises one or more amino acid mutations that facilitate the dimerization of the two arms of the Fc region. In certain embodiments, the first polypeptide comprises one or more of T366S, L368A and Y407V mutations (e.g., all of T366S, L368A and Y407V mutations) at the domain (e.g., CH3 domain) that forms the Fc region, and the second polypeptide comprises T366W mutation at the domain (e.g., CH3 domain) that forms the Fc region. In certain embodiments, the ILT3×CD3 binding agent has the configuration as depicted in F13 of FIG. 7. In some specific embodiments, the ILT3×CD3 binding agent provided herein comprises a first polypeptide comprising the amino acid sequence of SEQ ID NO:147, a second polypeptide comprising the amino acid sequence of SEQ ID NO: 169, a third polypeptide comprising the amino acid sequence of SEQ ID NO:20, and a fourth polypeptide comprising the amino acid sequence of SEQ ID NO:20.

### 5.4 Pharmaceutical Compositions

In another general aspect, provided is a pharmaceutical composition comprising an ILT3×CD3 binding agent provided herein and a pharmaceutically acceptable excipient. In another general aspect, provided is a pharmaceutical composition comprising a nucleic acid encoding the ILT3×CD3 binding agent provided herein or a fragment or a portion thereof and a pharmaceutically acceptable excipient. In another general aspect, provided is a pharmaceutical composition comprising an engineered cell expressing the ILT3×CD3 binding agent provided herein a pharmaceutically acceptable excipient.

In some embodiments, pharmaceutical compositions provided herein are prepared for storage by mixing the binding agent having the desired degree of purity with optional physiologically acceptable excipients (see, e.g., Remington, Remington's Pharmaceutical Sciences (18th ed. 1980)) in the form of aqueous solutions or lyophilized or other dried forms.

In another general aspect, provided herein is a method of producing a pharmaceutical composition comprising an antibody or antigen-binding fragment thereof provided herein, comprising combining an antibody or antigen-binding fragment thereof with a pharmaceutically acceptable carrier to obtain the pharmaceutical composition.

### 5.5 Methods of Use

The functional activity of binding agents provided herein can be characterized by methods known in the art and as described herein. Methods for characterizing binding agents include, but are not limited to, affinity and specificity assays including Biacore, ELISA, and OctetRed analysis; binding assays to detect the binding of antibodies to target cells by FACS; binding assays to detect the binding of antibodies to the target antigen on cells. According to particular embodiments, the methods for characterizing binding agents include those described below.

An ILT3×CD3 binding agent of the disclosure is useful in a variety of applications including, but not limited to, therapeutic treatment methods, such as treatment of cancer that expresses, human ILT3). In some embodiments, the therapeutic treatment methods comprise immunotherapy for cancer that expresses ILT3 (e.g., human ILT3). In some embodiments, an ILT3×CD3 binding agent is useful for activating, promoting, increasing, and/or enhancing an immune response to a cancer or cancer cells that express ILT3 (e.g., human ILT3). In some embodiments, an ILT3×CD3 binding agent is useful for activating, promoting, increasing, and/or enhancing an immune response to a tumor or tumor cells that express ILT3 (e.g., human ILT3). In some embodiments, an ILT3×CD3 binding agent is useful for activating, promoting, increasing, and/or enhancing a T cell response to a cancer or cancer cells that express ILT3 (e.g., human ILT3). In some embodiments, an ILT3×CD3 binding agent is useful for activating, promoting, increasing, and/or enhancing a T cell response to a tumor or tumor cells that express ILT3 (e.g., human ILT3). The methods of use may be in vitro, ex vivo, or in vivo methods.

In one aspect, provided herein is a method of directing a T cell to a cancer or tumor cell expressing ILT3 (e.g., human ILT3), comprising contacting the T cell with an effective amount of an ILT3×CD3 binding agent provided herein, wherein the CD3 binding region binds the T cell. In another aspect, provided herein is a method of directing a T cell to a cancer or tumor cell expressing ILT3 (e.g., human ILT3), comprising contacting the T cell with an effective amount of a pharmaceutical composition comprising an ILT3×CD3 binding agent provided herein, wherein the CD3 binding region binds the T cell. In some embodiments, the directed T cell induces apoptosis in the cancer or tumor cell. In some embodiments, when the T cell is directed to the cancer or tumor cell expressing ILT3 (e.g., human ILT3), the T cell induces differential cytotoxicity and cytokine release. That is, a method of directing a T cell to a cancer or tumor cell expressing ILT3 (e.g., human ILT3) results in T-cell dependent cytotoxicity (TDCC) that is inversely related to T cell cytokine release. For example, in some embodiments, TDCC is increased compared to a reference and cytokine release is decreased compared to a reference. In some embodiments, said TDCC reference is: (a) TDCC measured in a corresponding normal cell or issue; (b) TDCC measured in a neighboring non-cancerous cell or tissue in the same subject; or (c) TDCC measured in a corresponding cell or tissue measured in a cohort of healthy subjects. In some embodiments, said TDCC is determined by measuring apoptosis. In some embodiments, caspase mediated apoptosis is increased. In some embodiments, a cytokine reference is: (a) a cytokine measured in a corresponding normal cell or issue; (b) a cytokine measured in a neighboring non-cancerous cell or tissue in the same subject; or (c) a cytokine measured in a corresponding cell or tissue measured in a cohort of healthy subjects. In some embodiments, said cytokine release is determined by measuring TNFα. In some embodiments, TNFα cytokine release is decreased.

In some embodiments, the cancer or tumor cell comprises a hematological cancer or tumor cell. In some embodiments, the hematological cancer or tumor cell is an acute myeloid leukemia (AML) cell. In some embodiments, the AML is M4/M5 AML. In some embodiments, the cancer or tumor cell is an acute myeloid leukemia (AML) cell, a chronic myelomonocytic leukemia (CMML) cell, a B-cell acute lymphoblastic leukemia (B-ALL) cell, a chronic lymphocytic leukemia (CLL) cell, a diffuse large B-cell lymphoma (DLBCL) cell, a mantle cell lymphoma (MCL) cell, a multiple myeloma (MM) cell, a myelodysplastic syndrome (MDS) cell, a Hodgkin lymphoma cell, a lymphoplasmacytic lymphoma (LPL) cell, a follicular lymphoma cell, a Burkitt lymphoma cell, an blastic plasmacytoid dendritic cell neoplasm (BPDCN) cell, or a marginal zone lymphoma cell, or a mucosa-associated lymphoid tissue (MALT) lymphoma cell. In some embodiments, the cancer or tumor cell comprises a solid tumor cell.

In some embodiments, the cancer cell expresses a high level of ILT3 compared to a reference expression level. In some embodiments, the cancer cell expresses a low level of ILT3 compared to a reference expression level. In some embodiments, said reference expression level of ILT3 is: (a) a predetermined expression level of ILT3; (b) an ILT3 expression level in a corresponding normal cell or issue; (c) an ILT3 expression level measured in a neighboring non-cancerous cell or tissue in the same subject; or (d) an ILT3 expression level in a corresponding cell or tissue measured in a cohort of healthy subjects. In some embodiments, said expression level of ILT3 is determined by measuring the protein expression level of ILT3. In some embodiments, the cancer cell expresses a low level of ILT3 compared to a reference expression level, wherein the reference expression level is the ILT3 expression level in a known ILT3^{high} cancerous cell or tissue. Cancer cells express a low level of ILT3 include OCI-AML-2 or a NALM-1 cells. Cancer cells express a high level of ILT3 include M4 and M5 AML, MM, and B-ALL, THP-1, and MOLM13 cells.

In some embodiments, the binding agent provided herein does not induce T cell mediated killing of a normal bone marrow hematopoietic stem cell (HSC).

In one aspect, provided herein is a method of activating a T cell, comprising contacting the T cell with an effective amount of the ILT3×CD3 binding agent provided herein, wherein the CD3 binding region binds the T cell. In another aspect, provided herein is a method of activating a T cell, comprising contacting the T cell with a pharmaceutical composition comprising an ILT3×CD3 binding agent provided herein. In some embodiments, the T cell is a naïve T cell. In some embodiments, the T cell is polyclonally expanded from a population of PBMCs.

In one aspect, provided herein is a method of targeting an antigen on the surface of a target cell expressing ILT3 (e.g., human ILT-3), the method comprising contacting the target cell with an effective amount of an ILT3×CD3 binding agent provided herein, wherein the ILT3 binding region binds to the target cell. In another aspect, provided herein is a method of targeting an antigen on the surface of a target cell, the method comprising contacting the target cell with an effective amount of a pharmaceutical composition comprising an ILT3×CD3 binding agent provided herein, wherein the ILT3 binding region binds to the target cell. In some embodiments, provided herein is a method of targeting an antigen on the surface of a target cell, the method comprising contacting the target cell with an effective amount of a pharmaceutical composition comprising an ILT3×CD3 binding agent provided herein. In some embodiments, the target cell expresses a high level of ILT3 compared to a reference expression level. In some embodiments, the target cell expresses a low level of ILT3 compared to a reference expression level. In some embodiments, said reference expression level of ILT3 is: (a) a predetermined expression level of ILT3; (b) an ILT3 expression level in a corresponding normal cell or issue; (c) an ILT3 expression level measured in a neighboring non-cancerous cell or tissue in the same subject; or (d) an ILT3 expression level in a corresponding cell or tissue measured in a cohort of healthy subjects. In some embodiments, said expression level of ILT3 is determined by measuring the protein expression level of ILT3. In some embodiments, the target cell expresses a low level of ILT3 compared to a reference expression level, wherein the reference expression level is the ILT3 expression level in a known IL T3 high cancerous cell or tissue. Cancer cells express a low level of ILT3 include OCI-AML-2 or a NALM-1 cells. Cancer cells express a high level of ILT3 include M4 and M5 AML, MM, and B-ALL, THP-1, and MOLM13 cells. In some embodiments, the target cell is from a cancer (e.g., a hematological cancer). In some embodiments, the target cell comprises a cell from a B cell malignancy or a leukemia. In some embodiments, the cancer is acute myeloid leukemia (AML), including M4/M5 AML, chronic myelomonocytic leukemia (CMML), B-cell acute lymphoblastic leukemia (B-ALL), chronic lymphocytic leukemia (CLL), diffuse large B-cell lymphoma (DLBCL), mantle cell lymphoma (MCL), multiple myeloma (MM), myelodysplastic syndrome (MDS), Hodgkin lymphoma, lymphoplasmacytic lymphoma (LPL), follicular lymphoma, Burkitt lymphoma, blastic plasmacytoid dendritic cell neoplasm (BPDCN), or marginal zone lymphoma (e.g., mucosa-associated lymphoid tissue (MALT) lymphoma). In some embodiments, the cancer is a solid tumor.

In one aspect, provided herein is a method of killing or inhibiting the proliferation of a cancer or tumor cell expressing ILT3 (e.g. human ILT3), comprising contacting the cancer or tumor cell with an ILT3×CD3 binding agent provided herein. In another aspect, provided herein is a method of killing or inhibiting the proliferation of a cancer or tumor cell expressing ILT3 (e.g. human ILT3), comprising contacting the cancer or tumor cell with a pharmaceutical composition comprising an ILT3×CD3 binding agent provided herein. In some embodiments, the ILT3×CD3 binding agent activates a T cell. In some embodiments, the CD3 binding region activates the T cell. In some embodiments, the activated T cell induces apoptosis in the cancer cell or tumor cell. In some embodiments, the cancer or tumor cell comprises a hematological cancer or tumor cell. In some embodiments, the hematological cancer or tumor cell is an acute myeloid leukemia (AML) cell. In some embodiments, the AML is M4/M5 AML. In some embodiments, the cancer or tumor cell is an acute myeloid leukemia (AML) cell, a chronic myelomonocytic leukemia (CMML) cell, a B-cell acute lymphoblastic leukemia (B-ALL) cell, a chronic lymphocytic leukemia (CLL) cell, a diffuse large B-cell lymphoma (DLBCL) cell, a mantle cell lymphoma (MCL) cell, a multiple myeloma (MM) cell, a myelodysplastic syndrome (MDS) cell, a Hodgkin lymphoma cell, a lymphoplasmacytic lymphoma (LPL) cell, a follicular lymphoma cell, a Burkitt lymphoma cell, an blastic plasmacytoid dendritic cell neoplasm (BPDCN) cell, or a marginal zone lymphoma cell, or a mucosa-associated lymphoid tissue (MALT) lymphoma cell. In some embodiments, the cancer or tumor cell comprises a solid tumor cell. In some embodiments, the cancer or tumor cell expresses a high level of ILT3 compared to a reference expression level. In some embodiments, the cancer or tumor cell expresses a low level of ILT3 compared to a reference expression level. In some embodiments, said reference expression level of ILT3 is: (a) a predetermined expression level of ILT3; (b) an ILT3 expression level in a corresponding normal cell or issue; (c) an ILT3 expression level measured in a neighboring non-cancerous cell or tissue in the same subject; or (d) an ILT3 expression level in a corresponding cell or tissue measured in a cohort of healthy subjects. In some embodiments, said expression level of ILT3 is determined by measuring the protein expression level of ILT3. In some embodiments, the cancer cell expresses a low level of ILT3 compared to a reference expression level, wherein the reference expression level is the ILT3 expression level in a known IL T3 high cancerous cell or tissue. Cancer cells express a low level of ILT3 include OCI-AML-2 or a NALM-1 cells. Cancer cells express a high level of ILT3 include M4 and M5 AML, MM, and B-ALL, THP-1, and MOLM13 cells.

In one aspect, provided herein is a method of treating a cancer or tumor expressing ILT3 (e.g. human ILT3) in a subject, comprising administering an effective amount of an ILT3×CD3 binding agent provided herein. In another aspect, provided herein is a method of treating a cancer or tumor expressing ILT3 (e.g. human ILT3) in a subject, comprising administering an effective amount of a pharmaceutical composition comprising an ILT3×CD3 binding agent provided herein or the pharmaceutical composition provided herein. In some embodiments, the cancer or tumor is a hematological cancer or tumor. In some embodiments, the cancer or tumor is a leukemia. In some embodiments, the hematological cancer or tumor is acute myeloid leukemia (AML). In some embodiments, the cancer or tumor is a myelodysplastic syndrome. Myelodysplastic syndromes (MDS) are a group of cancers in which immature blood cells in the bone marrow do not mature and therefore do not become healthy blood cells. In some embodiments, myelodysplastic syndrome develops into AML. In some embodiments, the cancer or tumor is acute myeloid leukemia (AML), including M4/M5 AML, chronic myelomonocytic leukemia (CMML), B-cell acute lymphoblastic leukemia (B-ALL), chronic lymphocytic leukemia (CLL), diffuse large B-cell lymphoma (DLBCL), mantle cell lymphoma (MCL), multiple myeloma (MM), myelodysplastic syndrome (MDS), Hodgkin lymphoma, lymphoplasmacytic lymphoma (LPL), follicular lymphoma, Burkitt lymphoma, blastic plasmacytoid dendritic cell neoplasm (BPDCN), or marginal zone lymphoma (e.g., mucosa-associated lymphoid tissue (MALT) lymphoma). In some embodiments, the cancer or tumor comprises a solid tumor. In some embodiments, the cancer or tumor cell expresses a high level of ILT3 compared to a reference expression level. In some embodiments, the cancer or tumor cell expresses a low level of ILT3 compared to a reference expression level. In some embodiments, said reference expression level of ILT3 is: (a) a predetermined expression level of ILT3; (b) an ILT3 expression level in a corresponding normal cell or issue; (c) an ILT3 expression level measured in a neighboring non-cancerous cell or tissue in the same subject; or (d) an ILT3 expression level in a corresponding cell or tissue measured in a cohort of healthy subjects. In some embodiments, said expression level of ILT3 is determined by measuring the protein expression level of ILT3. In some embodiments, the cancer cell expresses a low level of ILT3 compared to a reference expression level, wherein the reference expression level is the ILT3 expression level in a known ILT3^{high} cancerous cell or tissue. Cancer cells express a low level of ILT3 include OCI-AML-2 or a NALM-1 cells. Cancer cells express a high level of ILT3 include M4 and M5 AML, MM, and B-ALL, THP-1, and MOLM13 cells.

In another aspect, provided herein is a use of the ILT3×CD3 binding agent provided herein in the manufacture of a medicament for treatment of a cancer or tumor expressing ILT3 (e.g. human ILT3) in a subject thereof. In yet a further aspect, provided herein is a binding agent for use in the treatment of a cancer or tumor expressing ILT3 (e.g. human ILT3). In some embodiments, the cancer or tumor is a hematological cancer or tumor. In some embodiments, the cancer or tumor is acute myeloid leukemia (AML), including M4/M5 AML, chronic myelomonocytic leukemia (CMML), B-cell acute lymphoblastic leukemia (B-ALL), chronic lymphocytic leukemia (CLL), diffuse large B-cell lymphoma (DLBCL), mantle cell lymphoma (MCL), multiple myeloma (MM), myelodysplastic syndrome (MDS), Hodgkin lymphoma, lymphoplasmacytic lymphoma (LPL), follicular lymphoma, Burkitt lymphoma, blastic plasmacytoid dendritic cell neoplasm (BPDCN), or marginal zone lymphoma (e.g., mucosa-associated lymphoid tissue (MALT) lymphoma). In some embodiments, the cancer or tumor is a myelodysplastic syndrome. Myelodysplastic syndromes (MDS) are a group of cancers in which immature blood cells in the bone marrow do not mature and therefore do not become healthy blood cells. In some embodiments, myelodysplastic syndrome develops into AML. In certain embodiments, the cancer or tumor comprises a hematological cancer. In some embodiments, the hematological cancer or tumor is acute myeloid leukemia (AML). In some embodiments, the AML is M4/M5 AML. In some embodiments, the cancer or tumor comprises a solid tumor.

In some embodiments, the cancer or tumor cell expresses a high level of ILT3 compared to a reference expression level. In some embodiments, the cancer or tumor cell expresses a low level of ILT3 compared to a reference expression level. In some embodiments, said reference expression level of ILT3 is: (a) a predetermined expression level of ILT3; (b) an ILT3 expression level in a corresponding normal cell or issue; (c) an ILT3 expression level measured in a neighboring non-cancerous cell or tissue in the same subject; or (d) an ILT3 expression level in a corresponding cell or tissue measured in a cohort of healthy subjects. In some embodiments, said expression level of ILT3 is determined by measuring the protein expression level of ILT3. In some embodiments, the cancer cell expresses a low level of ILT3 compared to a reference expression level, wherein the reference expression level is the ILT3 expression level in a known ILT3^{high} cancerous cell or tissue. Cancer cells express a low level of ILT3 include OCI-AML-2 or a NALM-1 cells. Cancer cells express a high level of ILT3 include M4 and M5 AML, MM, and B-ALL, THP-1, and MOLM13 cells.

In some embodiments, the subject is a subject in need thereof. In some embodiments, the subject is a human. In specific embodiments, the subject is administered an effective amount of the binding agent or pharmaceutical composition disclosed herein.

According to particular embodiments, the pharmaceutical compositions described herein are formulated to be suitable for the intended route of administration to a subject. For example, the pharmaceutical compositions described herein can be formulated to be suitable for intravenous, subcutaneous, or intramuscular administration.

In some embodiments, an ILT3×CD3 binding agent provided herein is used in combination with a supplemental therapy.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

### 5.6 Kits

In another general aspect, the disclosure relates to kits comprising an isolated bispecific antibody or antigen-binding fragment thereof provided herein and instructions for use.

In one embodiment, provided is a kit comprising an ILT3×CD3 binding agent provided herein. The described kits can be used to carry out the methods of using the ILT3×CD3 binding fragments provided herein, or other methods known to those skilled in the art. In some embodiments, the described kits can include the antibodies or antigen-binding fragments described herein and reagents for use in detecting the presence of an ILT3×CD3 binding agent in a biological sample. Accordingly, the described kits can include one or more of the antibodies, or an antigen-binding fragment(s) thereof, described herein and a vessel for containing the antibody or fragment when not in use, instructions for use of the antibody or fragment, the antibody or fragment affixed to a solid support, and/or detectably labeled forms of the antibody or fragment, as described herein.

In another embodiment, provided is a kit comprising the ILT3×CD3 binding agent comprising a first binding region specifically binding ILT3 and a second binding region specifically binding CD3 provided herein.

In some embodiments, the kit comprises an antibody described herein and reagents for detecting the antibody. The kit can further include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject.

In some embodiments, the kit comprises the ILT3×CD3 binding agent provided herein in a container and instructions for use of the kit.

In some embodiments, the ILT3×CD3 binding agent in the kit is labeled.

In case of conflict, the specification, including definitions, will control. As used herein and in the appended claims, the singular forms “a,” “an,” and “the” include plural referents unless the context clearly dictates otherwise. Thus, for example, reference to “a peptide sequence” or “a treatment,” includes a plurality of such sequences, treatments, and so forth. It is further noted that the claims can be drafted to exclude any optional element. As such, this statement is intended to serve as antecedent basis for use of such exclusive terminology such as “solely,” “only” and the like in connection with the recitation of claim elements, or use of a “negative” limitation.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges, and are also encompassed within the invention, subject to any specifically excluded limit in the stated range. Where the stated range includes one or both of the limits, ranges excluding either or both of those included limits are also included in the invention.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges, unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 96%, 97%, etc., as well as 91.1%, 91.2%, 91.3%, 91.4%, 91.5%, etc., 92.1%, 92.2%, 92.3%, 92.4%, 92.5%, etc., and so forth. In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1000, 1000-2500, 2500-5000, 5000-25,000, or 5000-50,000 includes any numerical value or range within or encompassing such values, e.g., 25, 26, 27, 28, 29 . . . 250, 251, 252, 253, 254 . . . 500, 501, 502, 503, 504 . . . , etc. The use of a series of ranges includes combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

Particular embodiments of this invention are described herein, including the best mode known to the inventors for carrying out the invention. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications, patent applications, accession numbers, and other references cited in this specification are herein incorporated by reference in its entirety as if each individual publication or patent application were specifically and individually indicated to be incorporated by reference. The publications discussed herein are provided solely for their disclosure prior to the filing date of the present application. Nothing herein is to be construed as an admission that the present invention is not entitled to antedate such publication by virtue of prior invention. Further, the dates of publication provided can be different from the actual publication dates which can need to be independently confirmed.

A number of embodiments of the invention have been described. Nevertheless, it will be understood that various modifications may be made without departing from the spirit and scope of the invention. Accordingly, the descriptions in the Experimental section are intended to illustrate but not limit the scope of invention described in the claims.

### 6. EMBODIMENTS

This invention provides the following non-limiting embodiments:

1. A binding agent comprising a first binding region that binds to human ILT3 and a second binding region that binds to human CD3, wherein the CD3 binding region comprises an anti-CD3 scFv.
2. The binding agent of embodiment 1, wherein the first binding region comprises an anti-ILT3 Fab.
3. The binding agent of embodiment 1 or 2, wherein the binding affinity of the first binding region for human ILT3 is higher than the binding affinity of the second binding region for human CD3.
4. The binding agent of embodiment 3, the binding affinity of the first binding region for human ILT3 is between about 10 folds and about 100 folds higher than the binding affinity of the second binding region for human CD3.
5. The binding agent of any one of embodiments 1-4, further comprises a Fc region.
6. The binding agent of embodiment 5, comprising:
(i) a first polypeptide comprising the anti-CD3 scFv, a first CH2 domain, and a first CH3 domain;
(ii) a second polypeptide comprising a VH domain of the first binding region, a CH1 domain, a second CH2 domain, and a second CH3 domain; and
(iii) a third polypeptide comprising a VL domain of the first binding region and a CL domain,
wherein the VH domain of the first binding region, the CH1 domain, the VL domain of the first binding region, and the CL domain form the anti-ILT3 Fab, and the first CH2 domain, the second CH2 domain, the first CH3 domain, and the second CH3 domain form the Fc region.
   7. The binding agent of embodiment 6, wherein the first polypeptide comprises one or more amino acid mutations that form an engineered cavity, and the second polypeptide comprising one or more amino acid mutations that form an engineered protuberance, and wherein the first polypeptide dimerizes with the second polypeptide via positioning of the protuberance into the cavity.
   8. The binding agent of any of embodiments 1-7, wherein the second binding region comprises a VH domain comprising a HCDR1, a HCDR2, and a HCDR3 of the amino acid sequence set forth in SEQ ID NO:149; and a VL domain comprising a LCDR1, a LCDR2, and a LCDR3 of the amino acid sequence set forth in SEQ ID NO:150.
   9. The binding agent of embodiment 8, wherein in the second binding region,
the VH domain of the second binding region comprises the HCDR1 comprising the amino acid sequence of SEQ ID NO:152, the HCDR2 comprising the amino acid sequence of SEQ ID NO: 153, and the HCDR3 comprising the amino acid sequence of SEQ ID NO: 154; and the VL domain of the second binding region comprises the LCDR1 comprising the amino acid sequence of SEQ ID NO:155, the LCDR2 comprising the amino acid sequence of SEQ ID NO:156, and the LCDR3 comprising the amino acid sequence of SEQ ID NO:157.
   10. The binding agent of any of embodiments 1-9, wherein the first binding region comprises a VH domain comprising a HCDR1, a HCDR2, and a HCDR3 of the amino acid sequence set forth in SEQ ID NO:17, and a VL domain comprising a LCDR1, a LCDR2, and a LCDR3 of the amino acid sequence set forth in SEQ ID NO:18.
   11. The binding agent of embodiment 10, wherein in the first binding region,
(a) the VH domain of the first binding region comprises the HCDR1 comprising the amino acid sequence of SEQ ID NO:1, the HCDR2 comprising the amino acid sequence of SEQ ID NO: 2, and the HCDR3 comprising the amino acid sequence of SEQ ID NO:3; and the VL domain of the first binding region comprises the LCDR1 comprising the amino acid sequence of SEQ ID NO: 4, the LCDR2 comprising the amino acid sequence of SEQ ID NO:5, and the LCDR3 comprising the amino acid sequence of SEQ ID NO:6;
(b) the VH domain of the first binding region comprises the HCDR1 comprising the amino acid sequence of SEQ ID NO:7, the HCDR2 comprising the amino acid sequence of SEQ ID NO: 8, and the HCDR3 comprising the amino acid sequence of SEQ ID NO:3; and the VL domain of the first binding region comprises the LCDR1 comprising the amino acid sequence of SEQ ID NO: 4, the LCDR2 comprising the amino acid sequence of SEQ ID NO:5, and the LCDR3 comprising the amino acid sequence of SEQ ID NO:6;
(c) the VH domain of the first binding region comprises the HCDR1 comprising the amino acid sequence of SEQ ID NO:1, the HCDR2 comprising the amino acid sequence of SEQ ID NO: 9, and the HCDR3 comprising the amino acid sequence of SEQ ID NO:3; and the VL domain of the first binding region comprises the LCDR1 comprising the amino acid sequence of SEQ ID NO: 4, the LCDR2 comprising the amino acid sequence of SEQ ID NO:5, and the LCDR3 comprising the amino acid sequence of SEQ ID NO:6;
(d) the VH domain of the first binding region comprises the HCDR1 comprising the amino acid sequence of SEQ ID NO:10, the HCDR2 comprising the amino acid sequence of SEQ ID NO: 2, and the HCDR3 comprising the amino acid sequence of SEQ ID NO:3; and the VL domain of the first binding region comprises the LCDR1 comprising the amino acid sequence of SEQ ID NO:4, the LCDR2 comprising the amino acid sequence of SEQ ID NO:5, and the LCDR3 comprising the amino acid sequence of SEQ ID NO:6; or
(e) the VH domain of the first binding region comprises the HCDR1 comprising the amino acid sequence of SEQ ID NO:11, the HCDR2 comprising the amino acid sequence of SEQ ID NO: 12, and the HCDR3 comprising the amino acid sequence of SEQ ID NO:13; and the VL domain of the first binding region comprises the LCDR1 comprising the amino acid sequence of SEQ ID NO:14, the LCDR2 comprising the amino acid sequence of SEQ ID NO:15, and the LCDR3 comprising the amino acid sequence of SEQ ID NO: 16.
   12. The binding agent of embodiment 10 or 11, wherein
(i) the first binding region comprises the VH domain having at least 90% sequence identity to the amino acid sequence of SEQ ID NO:17, and the VL domain having at least 90% sequence identity to the amino acid sequence of SEQ ID NO: 18; and the second binding region comprises the VH domain having at least 95% sequence identity to the amino acid sequence of SEQ ID NO: 149, and the VL domain having at least 90% sequence identity to the amino acid sequence of SEQ ID NO:150; or
(ii) the first binding region comprises the VH domain comprising the amino acid sequence of SEQ ID NO: 17, and the VL domain comprising the amino acid sequence of SEQ ID NO: 18; and the second binding region comprises the VH domain comprising the amino acid sequence of SEQ ID NO: 149, and the VL domain comprising the amino acid sequence of SEQ ID NO:150.
   13. A binding agent comprising a first binding region that binds to human ILT3 and a second binding region that binds to human CD3, wherein the second binding region comprises a VH domain comprising a HCDR1, a HCDR2, and a HCDR3 of the amino acid sequence set forth in SEQ ID NO:149, and a VL domain comprising a LCDR1, a LCDR2, and a LCDR3 of the amino acid sequence set forth in SEQ ID NO:150.
   14. The binding agent of embodiment 13, wherein the VH domain of the second binding region comprises the HCDR1 comprising the amino acid sequence of SEQ ID NO:152, the HCDR2 comprising the amino acid sequence of SEQ ID NO: 153, and the HCDR3 comprising the amino acid sequence of SEQ ID NO:154; and the VL domain of the second binding region comprises the LCDR1 comprising the amino acid sequence of SEQ ID NO: 155; the LCDR2 comprising the amino acid sequence of SEQ ID NO:156; and the LCDR3 comprising the amino acid sequence of SEQ ID NO: 157.
   15. The binding agent of embodiment 13 or 14, wherein the first binding region comprises a VH domain comprising a HCDR1, a HCDR2, and a HCDR3 of the amino acid sequence set forth in SEQ ID NO:17, and a VL domain comprising a LCDR1, a LCDR2, and a LCDR3 of the amino acid sequence set forth in SEQ ID NO:18.
   16. The binding agent of any one of embodiments 13-15, wherein in the first binding region,
(a) the VH domain of the first binding region comprises the HCDR1 comprising the amino acid sequence of SEQ ID NO:1, the HCDR2 comprising the amino acid sequence of SEQ ID NO: 2, and the HCDR3 comprising the amino acid sequence of SEQ ID NO:3; and the VL domain of the first binding region comprises the LCDR1 comprising the amino acid sequence of SEQ ID NO: 4, the LCDR2 comprising the amino acid sequence of SEQ ID NO:5, and the LCDR3 comprising the amino acid sequence of SEQ ID NO:6;
(b) the VH domain of the first binding region comprises the HCDR1 comprising the amino acid sequence of SEQ ID NO:7, the HCDR2 comprising the amino acid sequence of SEQ ID NO: 8, and the HCDR3 comprising the amino acid sequence of SEQ ID NO:3; and the VL domain of the first binding region comprises the LCDR1 comprising the amino acid sequence of SEQ ID NO: 4, the LCDR2 comprising the amino acid sequence of SEQ ID NO:5, and the LCDR3 comprising the amino acid sequence of SEQ ID NO:6;
(c) the VH domain of the first binding region comprises the HCDR1 comprising the amino acid sequence of SEQ ID NO:1, the HCDR2 comprising the amino acid sequence of SEQ ID NO: 9, and the HCDR3 comprising the amino acid sequence of SEQ ID NO:3; and the VL domain of the first binding region comprises the LCDR1 comprising the amino acid sequence of SEQ ID NO: 4, the LCDR2 comprising the amino acid sequence of SEQ ID NO:5, and the LCDR3 comprising the amino acid sequence of SEQ ID NO:6;
(d) the VH domain of the first binding region comprises the HCDR1 comprising the amino acid sequence of SEQ ID NO:10, the HCDR2 comprising the amino acid sequence of SEQ ID NO: 2, and the HCDR3 comprising the amino acid sequence of SEQ ID NO:3; and the VL domain of the first binding region comprises the LCDR1 comprising the amino acid sequence of SEQ ID NO:4, the LCDR2 comprising the amino acid sequence of SEQ ID NO:5, and the LCDR3 comprising the amino acid sequence of SEQ ID NO:6; or
(e) the VH domain of the first binding region comprises the HCDR1 comprising the amino acid sequence of SEQ ID NO:11, the HCDR2 comprising the amino acid sequence of SEQ ID NO: 12, and the HCDR3 comprising the amino acid sequence of SEQ ID NO:13; and the VL domain of the first binding region comprises the LCDR1 comprising the amino acid sequence of SEQ ID NO:14, the LCDR2 comprising the amino acid sequence of SEQ ID NO:15, and the LCDR3 comprising the amino acid sequence of SEQ ID NO:16.
   17. The binding agent of embodiment 15 or 16, wherein
(i) the first binding region comprises the VH domain having at least 90% sequence identity to the amino acid sequence of SEQ ID NO:17, and the VL domain having at least 90% sequence identity to the amino acid sequence of SEQ ID NO: 18; and the second binding region comprises the VH domain having at least 95% sequence identity to the amino acid sequence of SEQ ID NO: 149, and the VL domain having at least 90% sequence identity to the amino acid sequence of SEQ ID NO:150; or
(ii) the first binding region comprises the VH domain comprising the amino acid sequence of SEQ ID NO: 17, and the VL domain comprising the amino acid sequence of SEQ ID NO: 18; and the second binding region comprises the VH domain comprising the amino acid sequence of SEQ ID NO: 149, and the VL domain comprising the amino acid sequence of SEQ ID NO:150.
   18. The binding agent of any one of embodiments 13 to 17, wherein the first binding region comprises an anti-ILT3 Fab.
   19. The binding agent of any one of embodiments 13 to 18, wherein the second binding region comprises an anti-CD3 scFv.
   20. The binding agent of any one of embodiments 13 to 19, wherein the binding agent further comprises a Fc region.
   21. The binding agent of embodiment 20, wherein the binding agent comprises:
(i) a first polypeptide comprising the anti-CD3 scFv, a first CH2 domain, and a first CH3 domain;
(ii) a second polypeptide comprising the VH domain of the first binding region, a CH1 domain, a second CH2 domain and a second CH3 domain; and
(iii) a third polypeptide comprising the VL domain of the first binding region and a CL domain,
wherein the VH domain of the first binding region, the CH1 domain, the VL domain of the first binding region, and the CL domain form the anti-ILT3 Fab, and the first CH2 domain, the second CH2 domain, the first CH3 domain, and the second CH3 domain form the Fc region.
   22. The binding agent of embodiment 21, wherein the first polypeptide comprising one or more amino acid mutations that form an engineered cavity, and the second polypeptide comprising one or more amino acid mutations that form an engineered protuberance, and wherein the first polypeptide dimerizes with the second polypeptide via positioning of the protuberance into the cavity.
   23. The binding agent of embodiment 21 or 22, wherein
(i) the first polypeptide comprises the amino acid sequence of SEQ ID NO:147, the second polypeptide comprises the amino acid sequence of SEQ ID NO:19, and the third polypeptide comprises the amino acid sequence of SEQ ID NO:20, or
(ii) the first polypeptide comprises an amino acid sequence having at least 90% sequence identity of the amino acid sequence of SEQ ID NO:147, the second polypeptide comprises an amino acid sequence having at least 90% sequence identity of the amino acid sequence of SEQ ID NO:19, and the third polypeptide comprises an amino acid sequence having at least 90% sequence identity of the amino acid sequence of SEQ ID NO:20.
   24. The binding agent of embodiments 13 to 17, wherein the first binding region comprises two identical anti-ILT3 Fabs, and the second binding region comprises an anti-CD3 scFv.
   25. The binding agent of embodiment 24, wherein the binding agent comprises:
(i) a first polypeptide comprising the ant-CD3 scFv, a first CH2 domain, and a first CH3 domain;
(ii) a second polypeptide comprising a first VH domain, a second VH domain, a first CH1 domain, a second CH1 domain, a second CH2 domain, and a second CH3 domain, wherein each of the first and second VH domains comprises the VH domain of the first binding region;
(iii) a third polypeptide comprising a first VL domain and a first CL domain, wherein the first VL domain comprises the VL domain of the first binding region; and
(iv) a fourth polypeptide comprising a second VL domain and a second CL domain, wherein the second VL domain comprises the VL domain of the first binding region,
wherein the first VH domain and the first CH1 domain of the second polypeptide and the first VL domain and the first CL domain of the third polypeptide form a first Fab region, the second VH domain and the second CH1 domain of the second polypeptide and the second VL domain and the second CL domain of the fourth polypeptide form a second Fab region, and the first CH2 domain, the second CH2 domain, the first CH3 domain, and the second CH3 domain form the Fc region.
   26. The binding agent of embodiment 25, wherein the first polypeptide comprising one or more amino acid mutations that form an engineered cavity, and the second polypeptide comprising one or more amino acid mutations that form an engineered protuberance, and wherein the first polypeptide dimerizes with the second polypeptide via positioning of the protuberance into the cavity.
   27. The binding agent of embodiment 25 or 26, wherein
(i) the first polypeptide comprises the amino acid sequence of SEQ ID NO:147, the second polypeptide comprises the amino acid sequence of SEQ ID NO:169, the third polypeptide comprises the amino acid sequence of SEQ ID NO:20, and the fourth polypeptide comprises the amino acid sequence of SEQ ID NO:20; or
(ii) the first polypeptide comprises an amino acid sequence having at least 90% sequence identity of the amino acid sequence of SEQ ID NO:147, the second polypeptide comprises an amino acid sequence having at least 90% sequence identity of the amino acid sequence of SEQ ID NO: 169, the third polypeptide comprises an amino acid sequence having at least 90% sequence identity of the amino acid sequence of SEQ ID NO:20, and the fourth polypeptide comprises an amino acid sequence having at least 90% sequence identity of the amino acid sequence of SEQ ID NO: 20.
   28. The binding agent of any one of embodiments 1-12 and 19-27, wherein the anti-CD3 scFv comprises the amino acid sequence of SEQ ID NO:151.
   29. The binding agent of any one of embodiments 1 to 28, wherein the binding agent is a humanized antibody.
   30. A binding agent comprises:
(i) a first polypeptide comprising an scFv that binds to human CD3, a first CH2 domain, and a first CH3 domain;
(ii) a second polypeptide comprising a VH domain that binds to human ILT3, a CH1 domain, a second CH2 domain and a second CH3 domain; and
(iii) a third polypeptide comprising a VL domain that binds to human ILT3, and a CL domain,
wherein the scFv that binds to human CD3 comprises a VH domain comprising a HCDR1, a HCDR2, and a HCDR3 of the amino acid sequence set forth in SEQ ID NO:149, and a VL domain comprising a LCDR1, a LCDR2, and a LCDR3 of the amino acid sequence set forth in SEQ ID NO: 150; and
wherein the VH domain that binds to human ILT3 comprises a HCDR1, a HCDR2, and a HCDR3 of the amino acid sequence set forth in SEQ ID NO:17, and the VL domain that binds to human ILT3 comprises a LCDR1, a LCDR2, and a LCDR3 of the amino acid sequence set forth in SEQ ID NO:18.
   31. The binding agent of embodiment 30, wherein:
(a) the HCDR1 of the scFv comprises the amino acid sequence of SEQ ID NO: 152, the HCDR2 of the scFv comprises the amino acid sequence of SEQ ID NO: 153, the HCDR3 of the scFv comprises the amino acid sequence of SEQ ID NO:154, the LCDR1 of the scFv comprises the amino acid sequence of SEQ ID NO:155, the LCDR2 of the scFv comprises the amino acid sequence of SEQ ID NO: 156, and the LCDR3 of the scFv comprises the amino acid sequence of SEQ ID NO:157; and
(b) in the VH domain that binds to human ILT3 and the VL domain that binds to human ILT3
   (i) the HCDR1 comprises the amino acid sequence of SEQ ID NO:1; the HCDR2 comprises the amino acid sequence of SEQ ID NO:2; the HCDR3 comprises the amino acid sequence of SEQ ID NO:3; the LCDR1 comprises the amino acid sequence of SEQ ID NO:4; the LCDR2 comprises the amino acid sequence of SEQ ID NO:5; and the LCDR3 comprises the amino acid sequence of SEQ ID NO:6;
   (ii) the HCDR1 comprises the amino acid sequence of SEQ ID NO:7; the HCDR2 comprises the amino acid sequence of SEQ ID NO:8; the HCDR3 comprises the amino acid sequence of SEQ ID NO:3; the LCDR1 comprises the amino acid sequence of SEQ ID NO:4; the LCDR2 comprises the amino acid sequence of SEQ ID NO:5; and the LCDR3 comprises the amino acid sequence of SEQ ID NO:6;
   (iii) the HCDR1 comprises the amino acid sequence of SEQ ID NO: 1; the HCDR2 comprises the amino acid sequence of SEQ ID NO:9; the HCDR3 comprises the amino acid sequence of SEQ ID NO:3; the LCDR1 comprises the amino acid sequence of SEQ ID NO:4; the LCDR2 comprises the amino acid sequence of SEQ ID NO:5; and the LCDR3 comprises the amino acid sequence of SEQ ID NO:6;
   (iv) the HCDR1 comprises the amino acid sequence of SEQ ID NO: 10; the HCDR2 comprises the amino acid sequence of SEQ ID NO:2; the HCDR3 comprises the amino acid sequence of SEQ ID NO:3; the LCDR1 comprises the amino acid sequence of SEQ ID NO:4; the LCDR2 comprises the amino acid sequence of SEQ ID NO:5; and the LCDR3 comprises the amino acid sequence of SEQ ID NO:6; or
   (v) the HCDR1 comprises the amino acid sequence of SEQ ID NO:11; the HCDR2 comprises the amino acid sequence of SEQ ID NO:12; the HCDR3 comprises the amino acid sequence of SEQ ID NO: 13; the LCDR1 comprises the amino acid sequence of SEQ ID NO:14; the LCDR2 comprises the amino acid sequence of SEQ ID NO: 15; and the LCDR3 comprises the amino acid sequence of SEQ ID NO:16.
      32. The binding agent of embodiment 30 or embodiment 31, wherein the VH domain of the scFv that binds to human CD3 comprises the amino acid sequence of SEQ ID NO:149, and the VL domain of the scFv that binds to human CD3 comprises the amino acid sequence of SEQ ID NO: 150; and the VH domain that binds to human ILT3 comprises the amino acid sequence of SEQ ID NO: 17, and the VL domain that binds to human ILT3 comprises the amino acid sequence of SEQ ID NO:18.
      33. The binding agent of any one of embodiments 30 to 32, wherein the scFv comprises the amino acid sequence of SEQ ID NO: 151.
      34. An isolated polynucleotide encoding the binding agent of any one of embodiments to 33.
      35. A vector comprising the polynucleotide of embodiment 34.
      36. An isolated cell comprising the polynucleotide of embodiment 34 or the vector of embodiment 35.
      37. An isolated cell producing the binding agent of any one of embodiments 1 to 33.
      38. A pharmaceutical composition comprising the binding agent of any one of embodiments 1 to 33, the isolated polynucleotide of embodiment 34, the vector of embodiment 35, or the isolated cell of embodiment 36 or embodiment 37, and a pharmaceutically acceptable excipient.
      39. A method of directing a T cell to a cancer or tumor cell expressing ILT3, comprising contacting the T cell with an effective amount of the binding agent of any one of embodiments 1 to 33 or the pharmaceutical composition of embodiment 38.
      40. The method of embodiment 39, wherein the T cell induces the killing of the cancer cell or tumor cell expressing ILT3.
      41. The method of embodiment 40, wherein the cancer or tumor cell is a hematological cancer or tumor cell.
      42. The method of embodiment 41, wherein the hematological cancer or tumor cell is selected from the group consisting of an acute myeloid leukemia (AML) cell, a M4/M5 AML cell, a chronic myelomonocytic leukemia (CMML) cell, a B-cell acute lymphoblastic leukemia (B-ALL) cell, a chronic lymphocytic leukemia (CLL) cell, a diffuse large B-cell lymphoma (DLBCL) cell, a mantle cell lymphoma (MCL) cell, a multiple myeloma (MM) cell, a myelodysplastic syndrome (MDS) cell, a Hodgkin lymphoma cell, a lymphoplasmacytic lymphoma (LPL) cell, a follicular lymphoma cell, a Burkitt lymphoma cell, a blastic plasmacytoid dendritic cell neoplasm (BPDCN) cell, a marginal zone lymphoma cell, or a mucosa-associated lymphoid tissue (MALT) lymphoma cell.
      43. The method of any one of embodiments 39 to 42, wherein the T cell fails to induce killing of a normal hematopoietic stem cell (HSC).
      44. A method of activating a T cell, comprising contacting the T cell with an effective amount of the binding agent of any one of embodiments 1 to 33 or the pharmaceutical composition of embodiment 38, wherein the second binding region binds the T cell.
      45. The method of embodiment 44, wherein the T cell is a naïve T cell.
      46. The method of embodiment 44 or embodiment 45, wherein the T cell is polyclonally expanded from a population of PBMCs.
      47. A method of killing or inhibiting the proliferation of a cancer or tumor cell expressing ILT3, comprising contacting the cancer or tumor cell with the binding agent of any one of embodiments 1 to 33 or the pharmaceutical composition of embodiment 38.
      48. The method of embodiment 47, wherein the binding agent activates a T cell.
      49. The method of embodiment 48, wherein the activated T cell induces the killing of the cancer or tumor cell.
      50. The method of any one of embodiments 47 to 49, wherein the cancer or tumor cell comprises a hematological cancer or tumor cell.
      51. The method of embodiment 50, wherein the hematological cancer or tumor cell is selected from the group consisting of an acute myeloid leukemia (AML) cell, a M4/M5 AML cell, a chronic myelomonocytic leukemia (CMML) cell, a B-cell acute lymphoblastic leukemia (B-ALL) cell, a chronic lymphocytic leukemia (CLL) cell, a diffuse large B-cell lymphoma (DLBCL) cell, a mantle cell lymphoma (MCL) cell, a multiple myeloma (MM) cell, a myelodysplastic syndrome (MDS) cell, a Hodgkin lymphoma cell, a lymphoplasmacytic lymphoma (LPL) cell, a follicular lymphoma cell, a Burkitt lymphoma cell, a blastic plasmacytoid dendritic cell neoplasm (BPDCN) cell, a marginal zone lymphoma cell, or a mucosa-associated lymphoid tissue (MALT) lymphoma cell. 52. A method of treating a cancer or a tumor expressing ILT3 in a subject, comprising administering an effective amount of the binding agent of any one of embodiments 1 to 33 or the pharmaceutical composition of embodiment 38 to the subject.
      53. The method of embodiment 52, wherein the cancer or tumor comprises a hematological cancer or tumor.
      54. The method of embodiment 53, wherein the hematological cancer or tumor is selected from the group consisting of acute myeloid leukemia (AML), a M4/M5 AML chronic myelomonocytic leukemia (CMML), B-cell acute lymphoblastic leukemia (B-ALL), chronic lymphocytic leukemia (CLL), diffuse large B-cell lymphoma (DLBCL), mantle cell lymphoma (MCL), multiple myeloma (MM), myelodysplastic syndrome (MDS), Hodgkin lymphoma, lymphoplasmacytic lymphoma (LPL), follicular lymphoma, Burkitt lymphoma, blastic plasmacytoid dendritic cell neoplasm (BPDCN), marginal zone lymphoma, or mucosa-associated lymphoid tissue (MALT) lymphoma.

### 7. EXAMPLES

The following is a description of various methods and materials used in the studies, and are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (° C.), and pressure is at or near atmospheric. Standard abbreviations are used, including the following: bp=base pair(s); kb=kilobase(s); s or sec=second(s); min=minute(s); h or hr=hour(s); aa=amino acid(s); kb=kilobase(s); nt=nucleotide(s); pg=picogram; ng=nanogram; μg=microgram; mg=milligram; g=gram; kg=kilogram; pl or pL=picoliter(s); dl or dL=deciliter; μl or μL=microliter; ml or mL=milliliter; 1 or L=liter; μM=micromolar; mM=millimolar; M=molar; kDa=kilodalton; i.m.=intramuscular (ly); i.p.=intraperitoneal (ly); SC or SQ=subcutaneous (ly); QD=daily; BID=twice daily; QW=weekly; TIW=three times a week; QM=monthly; HPLC=high performance liquid chromatography; BW=body weight; U=unit; ns=not statistically significant; PBS=phosphate-buffered saline; PCR=polymerase chain reaction; NHS=N-Hydroxysuccinimide; HSA=human serum albumin; BSA=bovine serum albumin; DMEM=Dulbeco's Modification of Eagle's Medium; GC=genome copy; EDTA=ethylenediaminetetraacetic acid.

The following experimental methods were employed throughout the Examples described herein.

Protocol for generation of expanded T cells. T cells were purified from human PBMCs by negative selection using the pan T cell isolation kit (Miltenyi) and activated with Dynabeads coated with human anti-CD3/CD28 antibodies (Gibco/ThermoFisher Scientific) at a 1:1 cell:bead ratio in X-Vivo 15 media (Lonza) containing 5% normal human serum (MilliporeSigma), 2 mM GlutaGro (Corning), 10 mM HEPES (Corning), and 5 ng/ml IL-17, 5 ng/mL IL-15, and 25 ng/ml IL-2 (all from Peprotech) for 2 days. After the removal of the Dynabeads by passage over a magnetic column, the activated T cells were cultured for 8 more days in the same media and viably frozen in CryoStor CS10 cell preservation medium (StemCell Technologies) for future use in T-cell dependent cytotoxicity (TDCC) assays.

Protocol for generation of red fluorescent cell lines for use in T-cell dependent cytotoxicity (TDCC) assays. Target cell lines were stably transfected with red nuclear dye using Nuclight Red lentivirus reagent (Sartorius) at a multiplicity of infection (MOI) of 1-3. Red fluorescent cells were sorted on an Aria II flow cytometer and expanded.

Protocol for evaluating T-cell dependent cytotoxicity (TDCC) with expanded T cells. One day prior to the T cell toxicity assay, cryopreserved expanded T cells (generated as described above) were thawed and cultured in X-Vivo 15 media (Lonza) overnight. The next day, wells of 96-well, flat-bottom plates were pre-filled with 100 μL/well of serial dilutions of test antibody diluted in X-Vivo 15× to 4× the final concentration. A top final concentration of 1 μg/mL of each test article was used. Cryopreserved expanded T cells were viably thawed and resuspended to a density of 1×10⁶ cells/mL in X-Vivo 15 media and added to the plates at 50 μL/well. Fluorescently labeled cell lines were counted and resuspended to a density of 2× 10⁵ cells/mL in X-Vivo 15 media and added to the plates at 50 μL/well. The final effector:target cell ratio was 5:1. Caspase 3/7 Green Reagent (Sartorius), a caspase cleavage domain (DEVD) coupled to a green DNA-binding fluorescent label that is released upon DEVD cleavage by activated caspase 3/7, was added to the wells at a final concentration of 1:1000. The cultures were then imaged over a 24-hour period using an Incucyte ZOOM live cell imager (Sartorius). The percentage of apoptotic target cells was determined by the overlap of the red and green (caspase 3/7+) signals. Data was analyzed using the Incucyte ZOOM software, version 2018A (Sartorius). In some experiments, cell-free culture supernatants were harvested after 24 hours and analyzed for cytokine levels by Luminex (ProcartaPlex system; ThermoFisher Scientific).

Protocol for evaluating PBMC cytotoxicity. The wells of 96-well, flat-bottom plates were pre-filled with 100 μL/well of serial dilutions of test antibody diluted in X-Vivo 15 media (Lonza) to 4× the final concentration. A top final concentration of 10 μg/mL of each test article was used. Cryopreserved human PBMCs were viably thawed and resuspended to a density of 1×10⁶ cells/mL in X-Vivo 15 media and added to the plates at 50 μL/well. Fluorescently labeled cell lines were counted and resuspended to a density of 2× 10⁵ cells/mL in X-Vivo 15 media and added to the plates at 50 μL/well. The final effector:target cell ratio was 5:1. Caspase 3/7 Green Reagent (Sartorius), a caspase cleavage domain (DEVD) coupled to a green DNA-binding fluorescent label that is released upon DEVD cleavage by activated caspase 3/7, was added to the wells at a final concentration of 1:1000. The cultures were then imaged over a 24-hour period using an Incucyte ZOOM live cell imager (Sartorius) and supernatants were harvested for cytokine measurements by Luminex (ProcartaPlex system; ThermoFisher Scientific) after 24 hours. The percentage of apoptotic target cells was determined by the overlap of the red and green (caspase 3/7+) signals. Data was analyzed using the Incucyte ZOOM software, version 2018A (Sartorius).

Whole blood cytokine release assay. Immune activation by the antibodies and reference molecules was assessed by cytokine release assay. Antibodies were tested in both the plate-coated and soluble formats. For the plate-coated format, antibody dilutions were prepared at the indicated concentrations in PBS and added to 96-well flat-bottom tissue culture plates at 50 μL/well. Plates were incubated at 4° C. on a shaker at 300 rpm overnight, followed by two washes with PBS. For the soluble antibody format, antibody dilutions were prepared at 20× the indicated final concentration and 7.5 μL/well was added to the bottom of 96-well U-bottom tissue culture plates. Freshly collected, Na²⁺ Heparin-treated whole blood was added to the plates at 150 μl/well and the samples were incubated at 37° C. overnight. After centrifugation at 1800×g for 5 minutes at room temperature, plasma was collected from each well for cytokine analysis by Luminex assay (ProcartaPlex system; ThermoFisher Scientific). Reference molecules used included Staphylococcal enterotoxin B (1 μg/ml, soluble only), anti-CD3 (Biolegend), and an anti-CD28 superagonist antibody (clone ANC28.1, AnCell).

PBMC cytokine release assay. The cytokine release assay with PBMCs was performed similarly to the whole blood cytokine release assay except that cryopreserved PBMCs were viably thawed and plated at 2× 10⁵ cells/well in X-Vivo 15 media (Lonza) in a final volume of 150 μL/well.

T cell activation assay with primary AML samples. Cryopreserved bone marrow or PBMCs from M5 AML patients (Reprocell) were thawed in HBSS at room temperature and resuspended in RPMI 1640 (Corning) containing 10% heat-inactivated FBS, 1% GlutaGro (Corning), 50 mM β-mercaptoethanol (Gibco/ThermoFisher Scientific) and 1% penicillin-streptomycin (Corning). Primary AML cells (2× 10⁵ cells/well in 100 μL of media) were added to 100 μL of test antibodies prepared at 2× concentration in X-Vivo 15 media. Cells were incubated with test antibodies for 5 days at 37° C. On day 5, the cells were harvested and stained for 30 minutes at 4° C. with the fluorochrome-conjugated antibodies listed below. Data was collected on an LSR Fortessa flow cytometer (BD Biosciences) and analyzed using FlowJo software, v. 10.

| | Fluorophore | Vendor | Cat# | Isotype Cat# |
|---|---|---|---|---|
| CD45 | 805 | Becton Dickinson | 612891 | 612904 |
| CD34 | 395 | Becton Dickinson | 563778 | 563547 |
| CD3 | 650 | Becton Dickinson | 563999 | 563231 |
| CD4 | PerCP 5.5 | Becton Dickinson | 552838 | 522384 |
| CD25 | 605 | Biolegend | 302632 | 400161 |
| CD14 | PE | Biolegend | 367104 | 557436 |
| ILT3 | 647 | Biolegend | 333010 | 400130 |
| CD123 | 786 | Becton Dickinson | 751834 | 563330 |
| Sytox Blue | 421 | Life Technologies | S34857 | |

TDCC assay with primary CD34⁺ HSCs. One day prior to the T cell toxicity assay, cryopreserved expanded T cells (generated as described above) were thawed and cultured in X-Vivo 15 media (Lonza) overnight. On the day of the assay, cryopreserved CD34⁺ bone marrow cells (StemCell Technologies) were thawed in HBSS (Corning) at room temperature and resuspended in X-Vivo 15 media. Wells of 96-well plates were pre-filled with 100 μL of test antibodies at a 2× concentration. CD34⁺ hematopoietic stem cells (1×10⁴ cells/well in a 50 μL volume) and expanded T cells (5× 10⁴ cells/well in a 50 μL volume) were added to each well containing the test antibodies and Caspase 3/7 Green Reagent (Sartorius) was added at a final concentration of 1:1000. The cells were cultured overnight at 37° C. After centrifugation, the supernatants were collected for cytokine secretion analysis and the cells were stained for 30 minutes at 4° C. with antibodies against CD45, CD25, CD34, ILT3, and CD123 using the reagents listed above. Data was collected on an LSR Fortessa flow cytometer (BD Biosciences) and analyzed using FlowJo software, v. 10.

### 7.1 Example 1: Generation of ILT3×CD3 Bispecific Molecules

The purpose of this study was to design a cytotoxic T cell engager with enhanced selectivity for tumor cells and improved therapeutic index (i.e., a high affinity for binding to ILT3 expressing cells, and a good safety profile combined with efficient tumor cell killing). The safety of a T cell engager depends on minimizing cytokine release as well as maximizing tumor cell killing. The therapeutic index is measured using the ratio between tumor cell killing and cytokine release induced by the T cell engager. The greater the ratio, the better therapeutic index is for a T cell engager.

A panel of anti-ILT3 antibodies, and anti-CD3 antibodies with different binding affinities (high and low) were tested. The main criterion for selection of the ILT3 targeting arm of the bispecific was high affinity. The criterion for selecting a CD3 targeting arm was a good therapeutic index. Further, the design rationale was to have the ILT3 targeting arm binding to ILT3 with a 10× to 100× higher affinity than from the CD3 targeting arm binding to CD3, so that the T cell engager would bind the ILT3 expressing cancer cells before engaging any T cells. As a result, this design reduced off-target effect and increased safety profile. Tumor cell cytotoxicity and cytokine production were evaluated with the various antibody combinations and formats.

Selection of the ILT3 targeting arm. Various ILT3 antibody clones (see Tables 1-8) were coupled to either high affinity CD3 scFv (2B2) or low affinity CD3 scFv (1G4). The binding affinities of various ILT3 antibodies for ILT3 was shown in Table 9. T-cell dependent cellular cytotoxicity (TDCC) of each anti-ILT3 Fab when coupled to either CD3 scFv 2B2 or CD3 scFv 1G4 was shown in Table 9.

TABLE 9

| | | TDCC, EC₅₀ (nM) | |
|---|---|---|---|
| | | CD3 Arm | |
| ILT3 Arm | ILT3 Affinity (nM) | 2B2 (6 nM) | 1G4 (217 nM) |
| Hz45G10 | 0.13 | 0.008 | 1.26 |
| 53F10 | 0.25 | 0.013 | 2.0 |
| 48A6 | 0.08 | 0.025 | 0.50 |
| 3A3 | 2.0 | 0.40 | 50.12 |
| 16C5 | 0.7 | 3.16 | 50.12 |
| 12A12 | 8.5 | 3.16 | 251.2 |

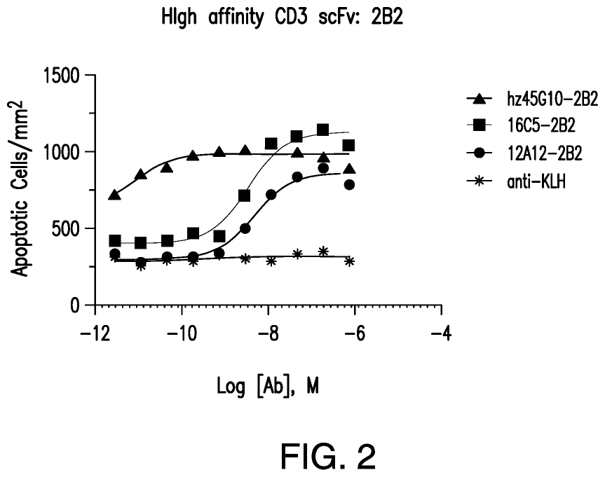

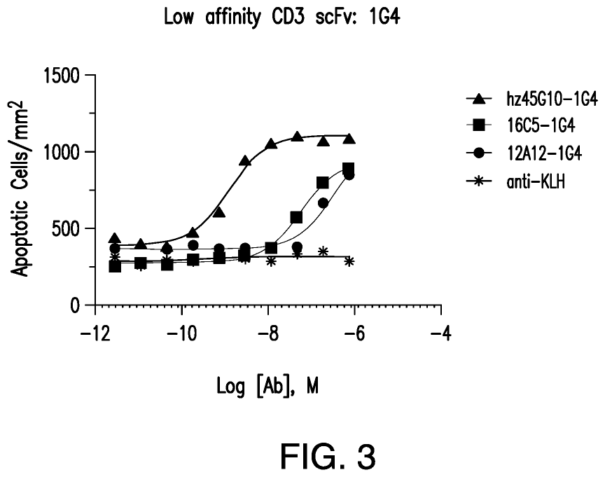

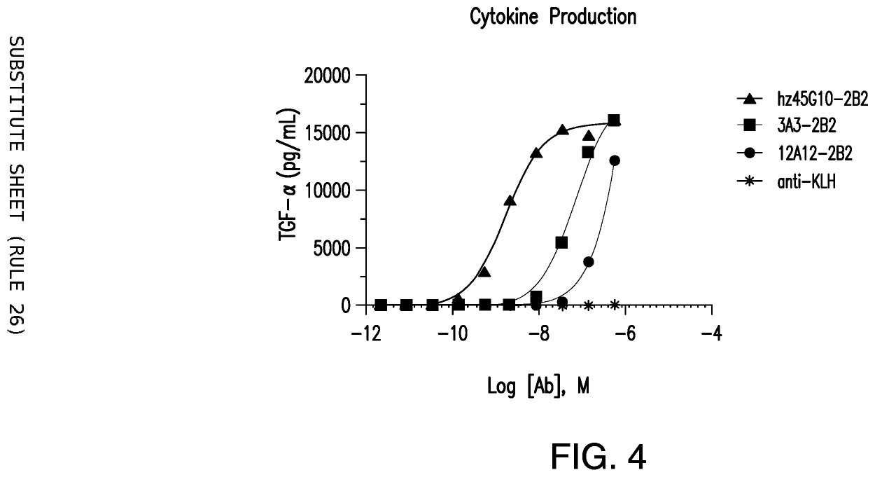

All ILT3×CD3 bispecific molecules tested induced TDCC and cytokine release. Among all the anti-ILT3 clones test, Hz45G10 had high affinity for ILT3. Hz45G10 when coupled with CD3 scFv 2B2 (FIG. 2) or CD3 scFv 1G4 (FIG. 3) potently induced apoptosis compared to other ILT3 antibody clones (16C5 or 12A12). Furthermore, Hz45G10 Fab when coupled with CD3 scFv 2B2 induced low TNFα release (FIG. 4, Table 10). Altering the ILT3 antibody had no effect on the ratio between cytotoxicity and cytokine production (see Table 10).

TABLE 10

| | TDCC, | ILT3 | TNF-α, | Ratio |
|---|---|---|---|---|
| ILT3 Arm | EC₅₀ (nM) | Affinity (nM) | EC₅₀ (nM) | (TDCC/TNF) |
| Hz45G10 | 0.008 | 0.13 | 1.58 | 0.005 |
| 53F10 | 0.013 | 0.25 | 2.0 | 0.007 |
| 48A6 | 0.025 | 0.08 | 2.51 | 0.010 |
| 3A3 | 0.4 | 2.0 | 79.4 | 0.005 |
| 16C5 | 3.16 | 0.7 | 398.1 | 0.008 |
| 12A12 | 3.16 | 8.5 | 2511.9 | 0.001 |

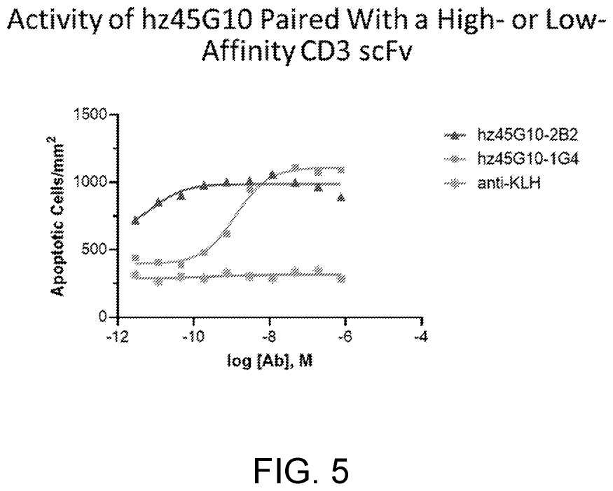

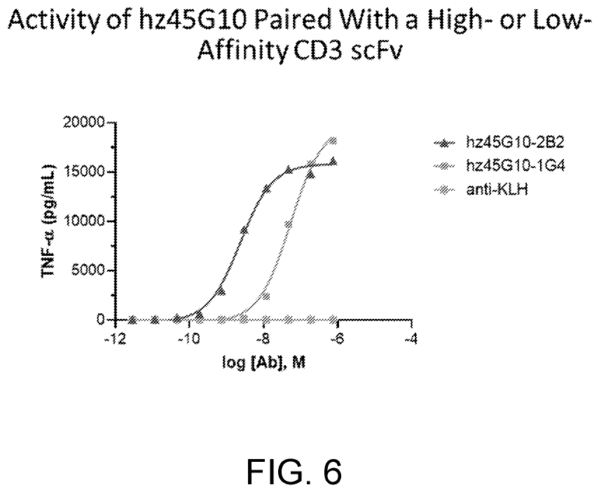

Selection of the CD3 targeting arm. The affinity of the CD3 binding arm of a T cell engager can vary depending on the tumor antigen binding arm. For the ILT3-CD3 T cell engager, an optimal CD3 scFv was produced based on the selected Hz45G10 clone. First, two CD3 scFv clones with different CD3 binding affinity were tested (CD3 scFv 2B2 with a high affinity for CD3, and r CD3 scFv 1G4 with a low affinity for CD3, see International Publication No. WO 2008/119567 and U.S. Pat. No. 10,066,016) when coupled with Hz45G10. CD3 scFv 2B2 showed a 35-fold higher affinity for CD3 than scFv 1G4. 2B2 scFv showed 160 fold increase of T-cell dependent cellular cytotoxicity (TDCC) compared to scFv IG4, but only increased the cytokine production 20-fold. See FIG. 5, FIG. 6 and Table 11.

TABLE 11

| | |
|---|---|
| TDCC, EC₅₀ (nM) | TNF-α Secretion, EC₅₀ (nM) |
| CD3 Arm | CD3 Arm |

| 2B2 (6 nM) | 1G4 (217 nM) | 2B2 (6 nM) | 1G4 (217 nM) |
|---|---|---|---|
| 0.008 | 1.26 | 50.12 | 2.51 |

In view of the above results, an anti-CD3 scFv clone with high binding affinity for CD3 was developed (VH as SEQ ID NO: 149, VL as SEQ ID NO:150, K_{D}=6 nM determined by SPR). The binding affinity of Hz45G10 for human ILT3 was 38 fold higher affinity than the CD3 scFv.

Next, a bispecific antibody incorporating Hz45G10 and the CD3 scFv was generated in a variety of formats as indicated in FIG. 7 and evaluated. All bispecific formats were screened in cytotoxicity and cytokine release assays.

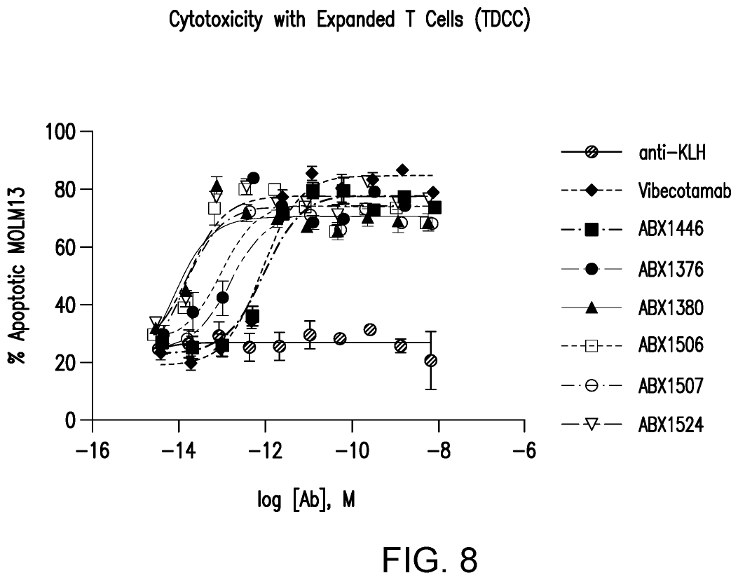

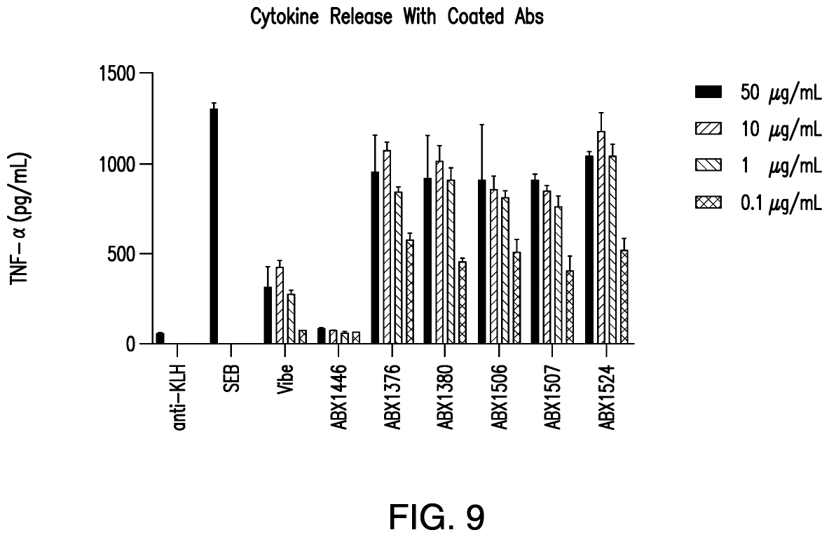

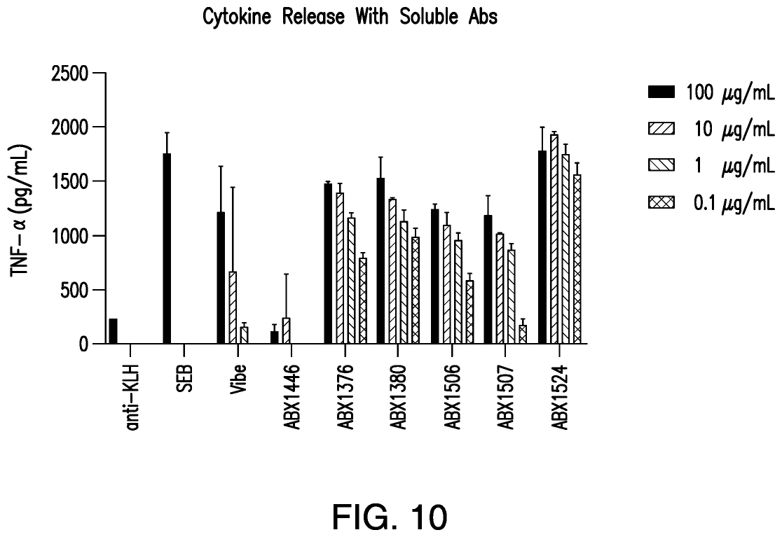

The F0 (i.e., ABX1446) ILT3×CD3 bispecific antibody format showed strong cytolytic activity (See Table 12 and FIG. 8) in the AML cell line, MOLM13. The F0 ILT3×CD3 bispecific antibody format also induced low cytokine secretion in a whole blood cytokine release assay when whole blood was added to plates pre-coated with F0 (FIG. 9) or added to culture medium with soluble F0 (FIG. 10). See Table 13. Similarly, among the test formats, F13 (i.e., ABX1520) showed strong cytolytic activity but low cytokine secretion.

TABLE 12

| | Format | TDCC, EC₅₀ (pM) |
|---|---|---|
| Vibecotamab | | 0.8 |
| ABX1446 | F0 | 0.9 |
| ABX1376 | F2 | 0.09 |
| ABX1380 | F1 | 0.007 |
| ABX1506 | F6 | 0.01 |
| ABX1507 | F5 | 0.15 |
| ABX1524 | F7 | 0.02 |

TABLE 13

| | Coated Ab | | Soluble Ab | |
|---|---|---|---|---|
| | EC₅₀, | TNF-α at 10 | EC₅₀, | TNF-α at 10 |
| | TNF-α | μg/mL Ab | TNF-α | μg/mL Ab |
| Format | (nM) | (pg/mL) | (nM) | (pg/mL) |
| Vibecotamab | 2.05 | 422.5 | ND | 1217.9 |
| F0 | 114.0 | 73.3 | | 112.3 |
| F2 | 3.07 | 1075.3 | | 1475.4 |
| F1 | 0.027 | 1012.9 | | 1524.5 |
| F6 | 0.524 | 856.7 | | 1235.2 |
| F5 | 1.386 | 852.7 | | 1185.6 |
| F7 | 0.016 | 1176.1 | | 1784.9 |

### 7.2 Example 2: In Vitro Characterization of ILT3×CD3 Bispecific Molecules

The purpose of this study was to evaluate the in vitro activity of F0 (i.e., ABX1446) and F13 (i.e., ABX1520) in cell lines expressing ILT3. Cell toxicity assays and cytokine secretions were performed as outlined in Example 1.

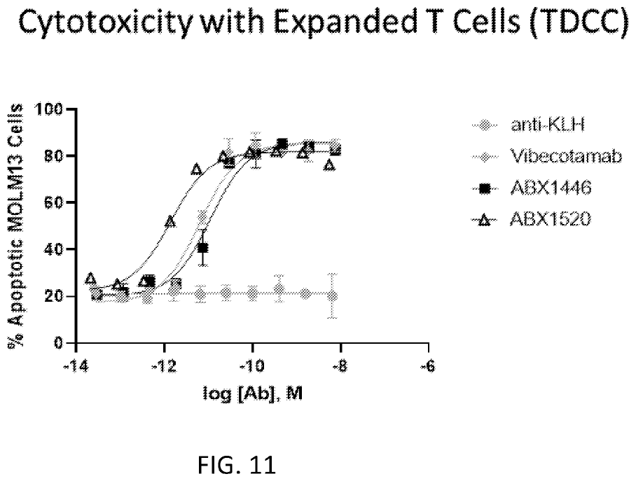

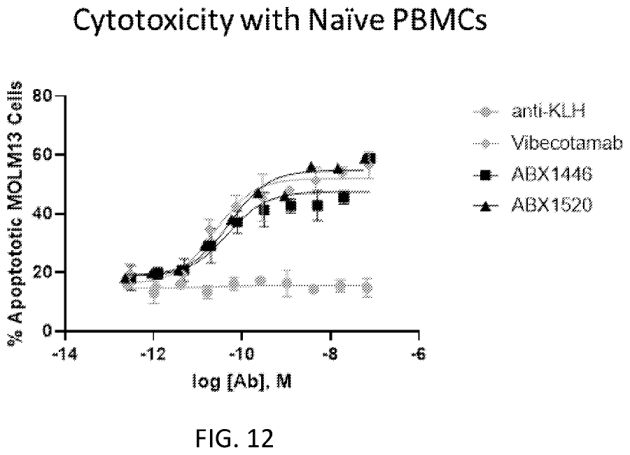

ABX1446 and ABX1520 potently induced apoptosis in ILT3 positive (ILT3⁺) AML cells (MOLM13) when expanded T cells (FIG. 11) or naïve T cells (from PBMCs; FIG. 12) were used as effectors. See Table 14.

TABLE 14

| | | PBMC Cytotoxicity, |
|---|---|---|
| | TDCC, EC₅₀ (pM) | EC₅₀ (pM) |
| Vibecotamab | 6.3 | 24.5 |
| ABX1446 | 10.9 | 46.8 |
| ABX1520 | 1.4 | 53.7 |

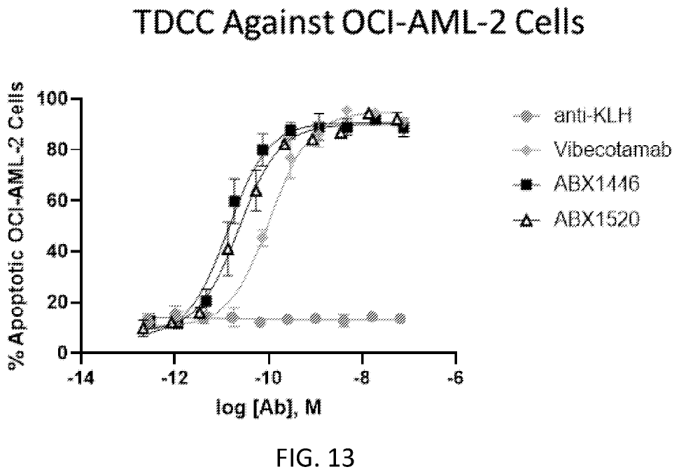

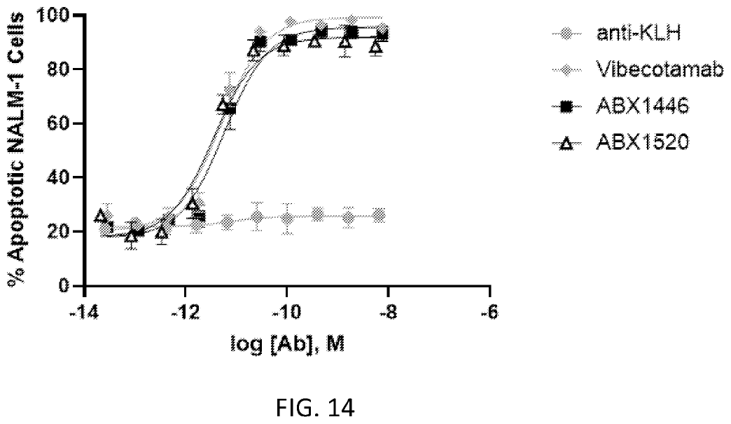

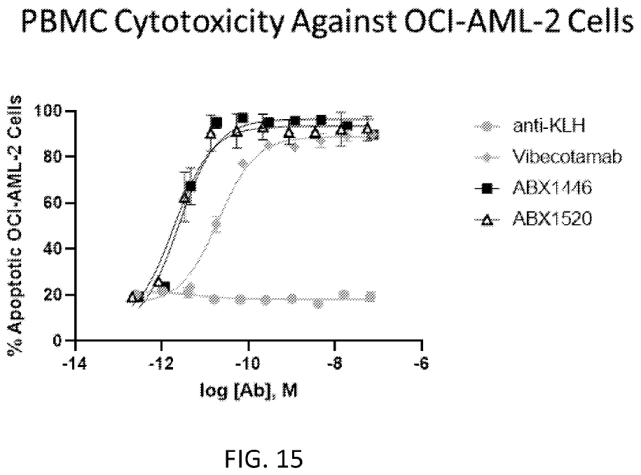

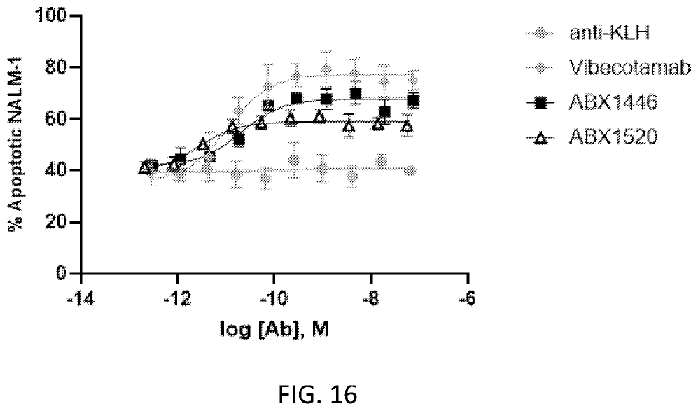

ABX1446 and ABX1520 potently induced apoptosis in AML cells with low expression of ILT3 (OCI-AML-2 and NALM-1 cells) when expanded T cells (FIG. 13, FIG. 14 and Table 15) or naïve T cells (from PBMCs; FIG. 15, FIG. 16 and Table 16) were used as effectors.

TABLE 15

| | TDCC vs. OCI-AML-2 Cells, | TDCC vs. NALM-1 Cells, |
|---|---|---|
| | EC₅₀ (pM) | EC₅₀ (pM) |
| Vibecotamab | 106.2 | 5.0 |
| ABX1446 | 13.5 | 5.7 |
| ABX1520 | 23.3 | 3.6 |

TABLE 16

| | TDCC vs. OCI-AML-2 Cells, | TDCC vs. NKM-1 Cells, |
|---|---|---|
| | EC₅₀ (pM) | EC₅₀ (pM) |
| Vibecotamab | 20.0 | 11.2 |
| ABX1446 | 2.5 | 20.4 |
| ABX1520 | 2.0 | 2.6 |

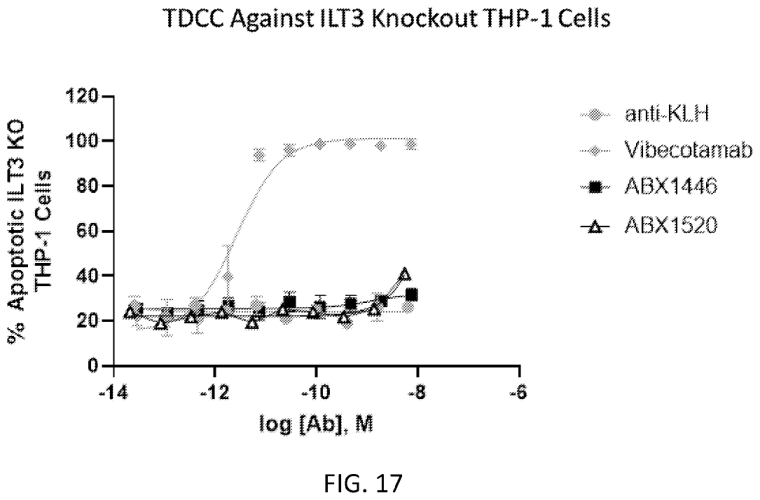

ABX1446 and ABX1520 failed to induce TDCC against ILT3 knockout THP-1 cells. See FIG. 17 and Table 17.

TABLE 17

| | TDCC vs. ILT3 Knockout |
|---|---|
| | THP-1 Cells, EC₅₀ (pM) |
| Vibecotamab | 2.7 |
| ABX1446 | |
| ABX1520 | — |

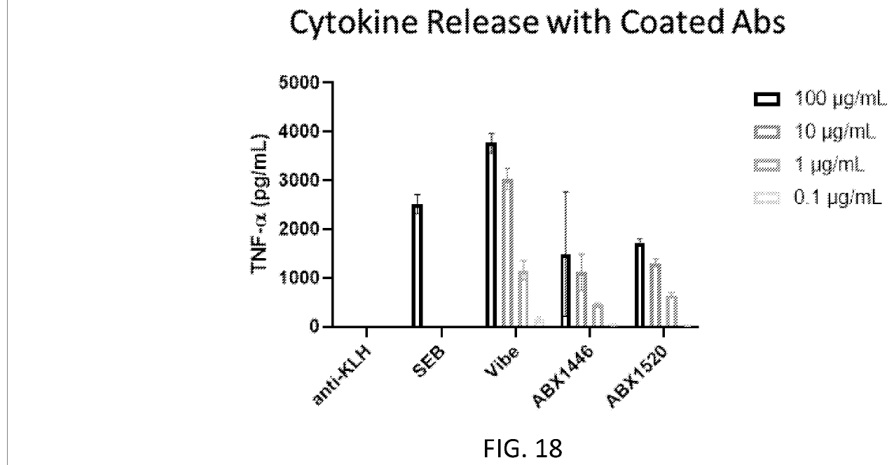

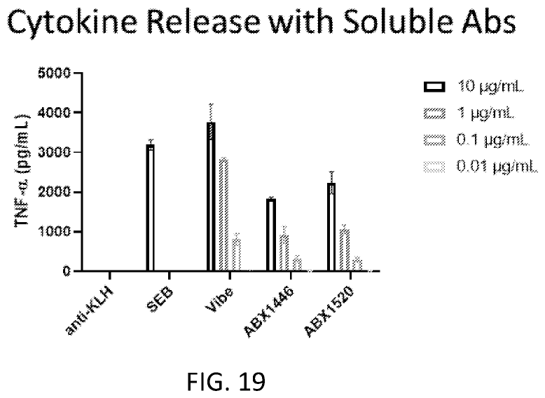

ABX1446 and ABX1520 induced low cytokine release in a PBMC cytokine secretion assay when PBMCs were incubated with ABX1446 or ABX1520 in a plate-coated format (FIG. 18) or in a soluble format (FIG. 19). See Table 18.

TABLE 18

| | |
|---|---|
| Coated Ab | Soluble Ab |

| | | TNF-α at | | TNF-α at |
|---|---|---|---|---|
| | EC₅₀, | 10 μg/mL | EC₅₀, | 10 μg/mL |
| | TNF-α | Ab | TNF-α | Ab |
| | (nM) | (pg/mL) | (nM) | (pg/mL) |
| Vibecotamab | 18 | 3764.9 | 35.3 | 3777.1* |
| ABX1446 | 21.5 | 1492.9 | 42.5 | 1837.2** |
| ABX1520 | 9.4 | 1726.8 | 45.8 | 2240.0*** |

*Vibecotamab: 10 μg/mL Ab = 74.62 nM
**ABX1446:: 10 μg/mL Ab = 80.65 nM
***ABX1520:: 10 μg/mL Ab = 55.2 nM

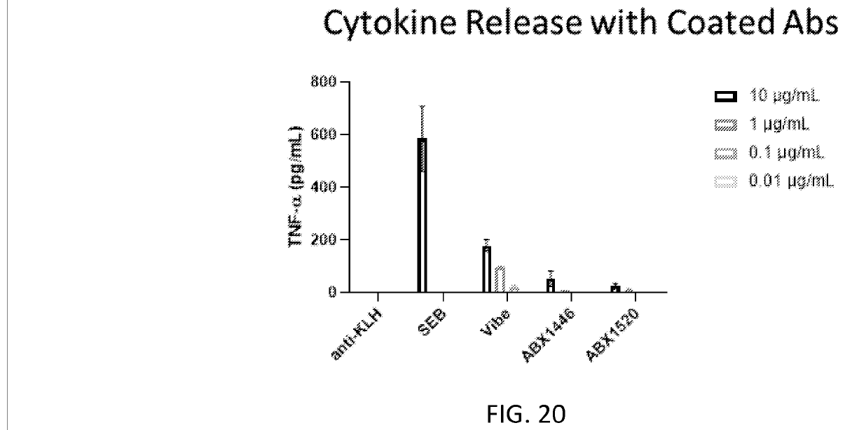

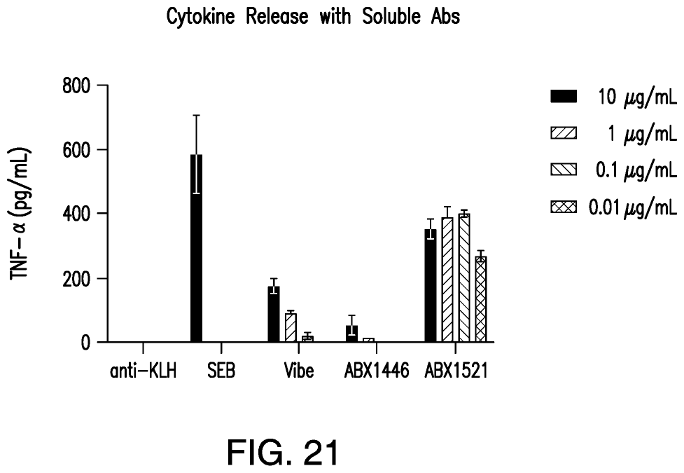

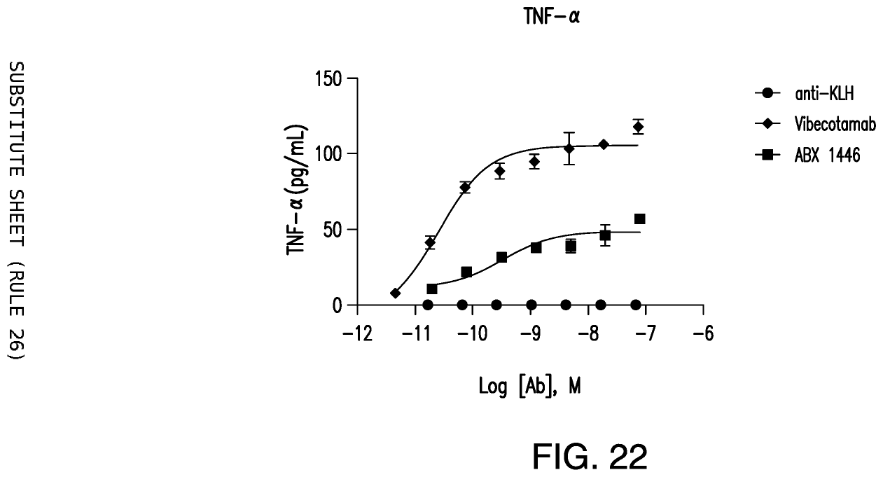

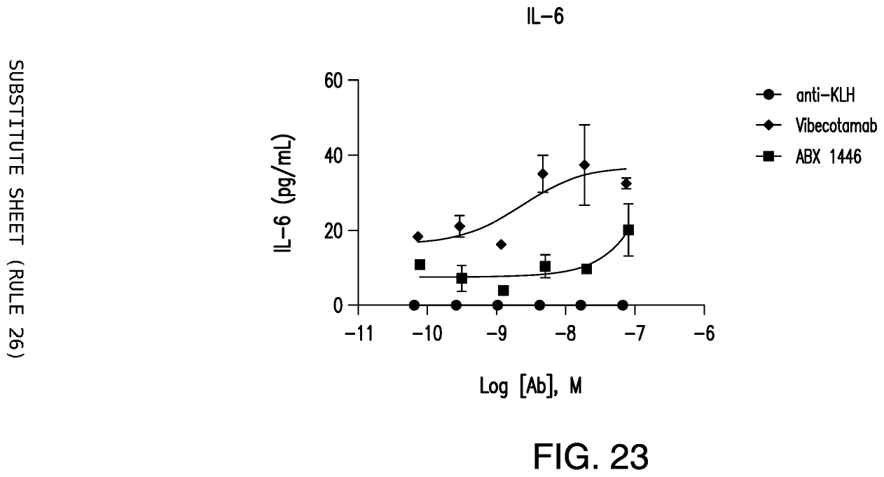

ABX1446 and ABX1520 induced low cytokine release in a whole blood cytokine secretion assay when whole blood was incubated with ABX1446 in a plate-coated format (FIG. 20) or in a soluble format (FIG. 21). See Table 19. Additionally, although ABX1446 and vibecotamab showed similar potency in PBMC cytotoxicity assays, ABX1446 induced low cytokine secretion in PBMC cytotoxicity assays. See FIG. 22, FIG. 23 and Table 20.

TABLE 19

Coated Ab

| | EC₅₀, TNF-α | TNF-α at 10 μg/mL Ab |
|---|---|---|
| Donor 830 | (nM) | (pg/mL) |
| Vibecotamab | 2.05 | 422.5* |
| ABX1446 | 114.0 | 73.3** |
| ABX1376 | 3.07 | 1075.3 |

Soluble Ab

| | EC₅₀, TNF-α | TNF-α at 10 μg/mL Ab |
|---|---|---|
| Donor 830 | (nM) | (pg/mL) |
| Vibecotamab | ND | 179.3* |
| ABX1446 | ND | 53.1** |
| ABX1376 | ND | 356.3 |
| ABX1520 | ND | 24.6*** |

TABLE 20

| | EC₅₀, | | | EC₅₀, |
|---|---|---|---|---|
| | TNF-α | EC₅₀, IL-6 | EC₅₀, IL-2 | IFN-γ |
| | (nM) | (nM) | (nM) | (nM) |
| Vibecotamab | 0.03 | 2.5 | 0.1 | 0.03 |
| ABX1446 | 0.3 | 7933.3 | 0.1 | 0.4 |

*Vibecotamab: 10 μg/mL Ab = 74.62 nM
**ABX1446: 10 μg/mL Ab = 80.65 nM
*** ABX1520: 10 μg/mL Ab = 55.2 nM

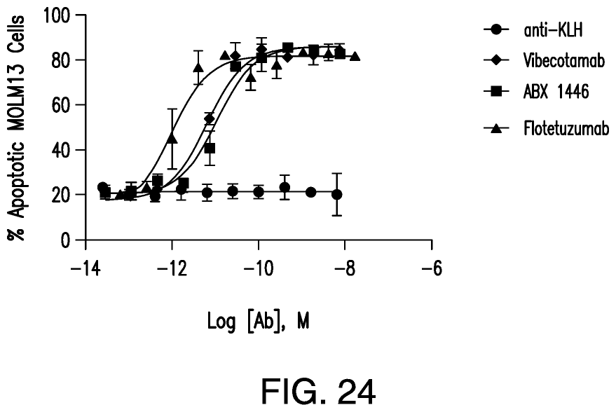

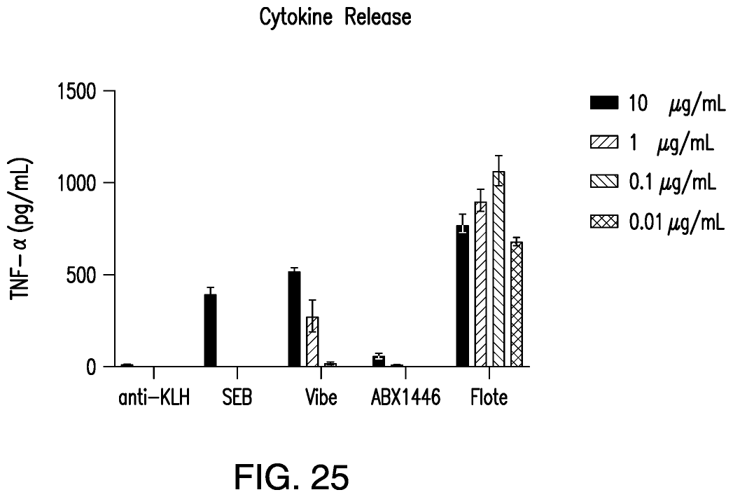

When compared to a CD123×CD3 DART (Flotetuzumab), ABX1446 induced low cytokine secretion (FIG. 25) but induced potent apoptosis in MOLM13 cells (FIG. 24). ABX1446 induced less cytokine secretion compared to Flotetuzumab. See Table 21.

TABLE 21

Cytokine Release

| | | EC₅₀, | TNF-α at 10 |
|---|---|---|---|
| | | TNF-α | μg/mL Ab |
| | TDCC, EC₅₀ (pM) | (nM) | (pg/mL) |
| Vibecotamab | 6.5 | −7.56 | 518.1 |
| Flotetuzumab | 0.97 | ND | 778.2 |
| ABX1446 | 11 | −7.1 | 61.5 |

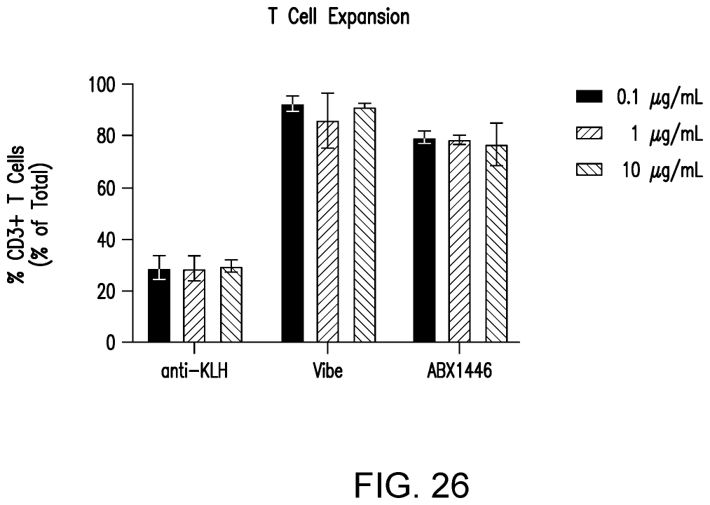

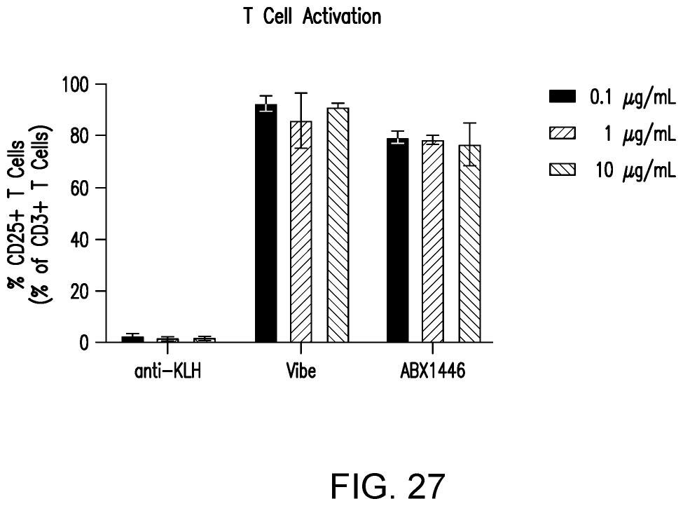

In order to study T cell expansion and activation in PBMCs isolated from M5 AML patients, PBMCs were incubated with ABX1446, and expansion and activation measured by flow cytometry. ABX1446 induced T cell expansion (FIG. 26) and activation (FIG. 27) in M5 PBMCs.

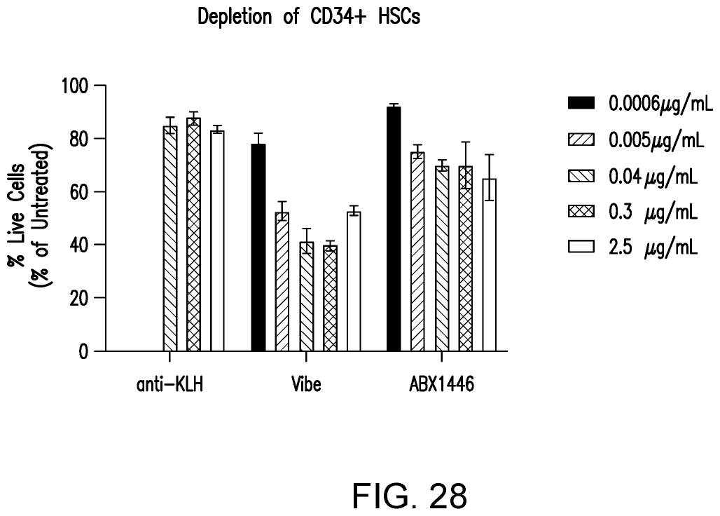

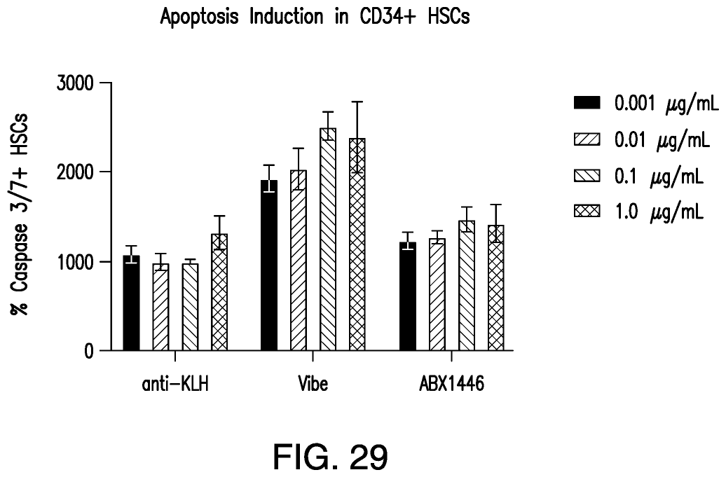

ABX1446 failed to induce depletion (FIG. 28) and apoptosis (FIG. 29) against primary HSCs.

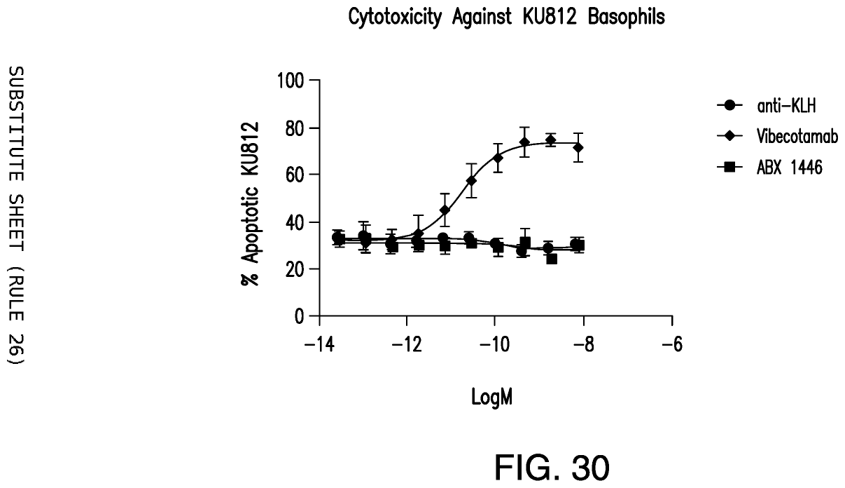

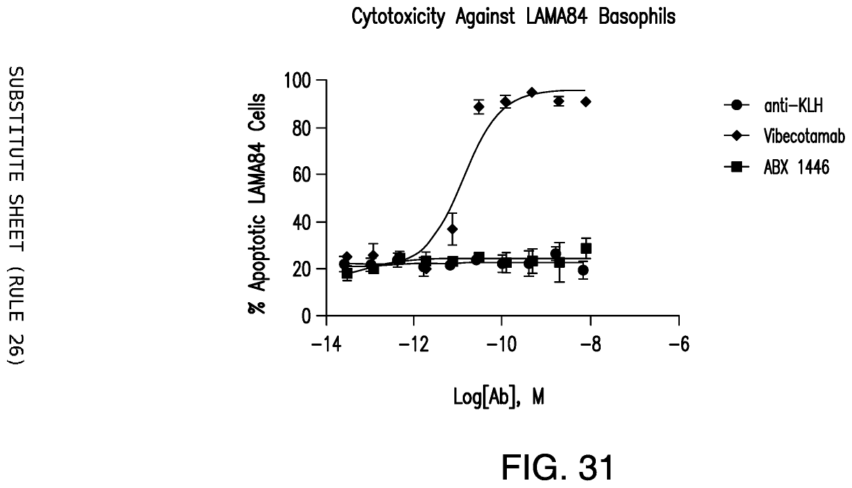

Further, ABX1446 failed to ablate non-monocytic immune cells. CD123 is expressed on many immune cell types, while ILT3 is expressed only on a subset. Use of bispecific antibody Vibecotamab could represent a safety risk in the clinical setting. Thus, cytotoxicity in KU812 basophils and LAMA84 basophils was measured. Both cell types are CD123-positive and ILT3-negative. Thus, ABX1446 had no effect on these basophils. See FIG. 30 and FIG. 31.

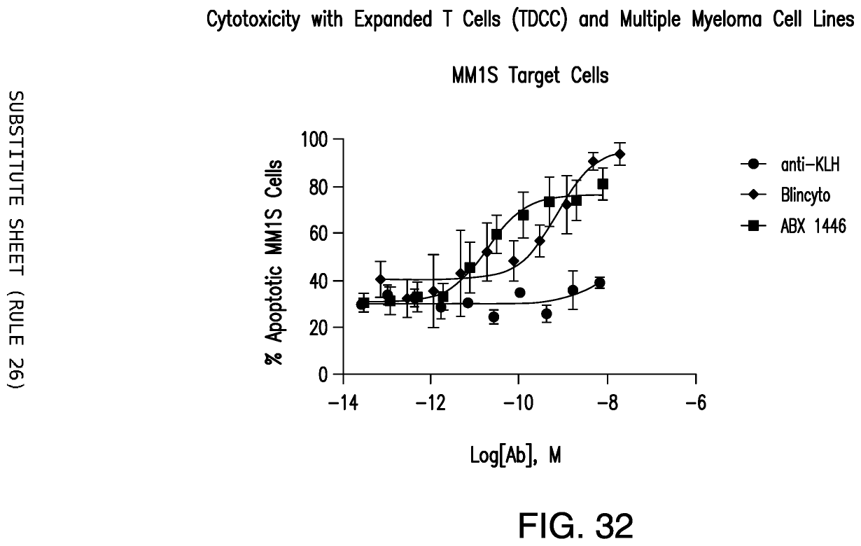

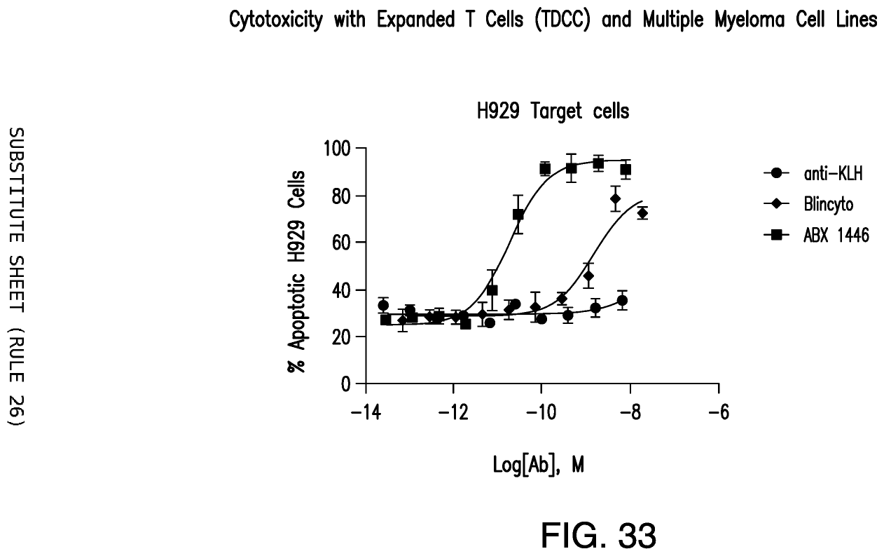

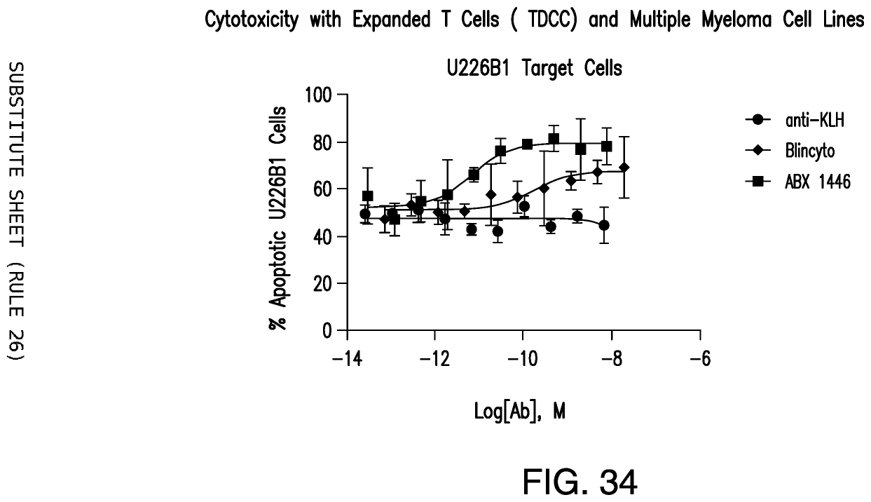

Multiple myeloma cells also express ILT3. The F0 ILT3×CD3 bispecific antibody format (ABX1446) showed strong cytolytic activity against MM1S (FIG. 32), H929 (FIG. 33) and U226B1 (FIG. 34) multiple myeloma cell lines. See Table 22.

TABLE 22

| | MM1S | H929 | U226B1 |
|---|---|---|---|
| | TDCC, EC₅₀ (pM) | | |
| Blincyto | 704.7 | 1503.1 | 163.7 |
| ABX1446 | 18.6 | 18.62 | 6.5 |

The above described studies demonstrated that ABX1446 showed potent cytotoxicity with low levels of cytokine release compared to T cell engager bispecific Vibecotamab. Additionally, ABX1446 did not ablate HSCs or mature immune cells. Thus, ABX1446 had a safety profile that distinguishes it from current T cell engage bispecific antibodies on the market.

### 7.3 Example 3: In Vivo Characterization of ILT3×CD3 Bispecific Molecules

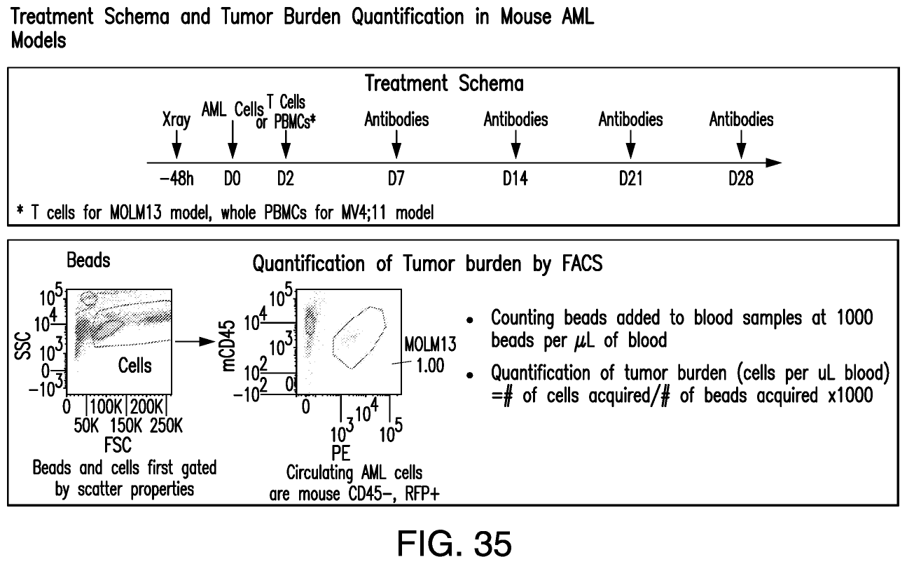

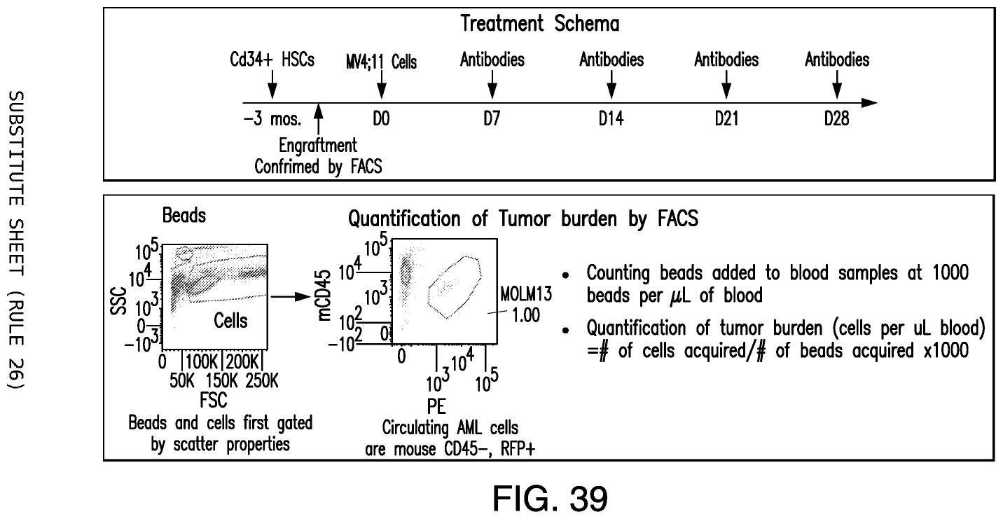

The purpose of this study was to evaluate the in vivo activity of F0 (i.e., ABX1446) and F13 (i.e., ABX1520) in three human AML mouse models, including a MOLM13 AML model (FIG. 35), an MV4; 11 AML model (FIG. 35), and a model with CD34⁺ humanized mice engrafted with MV4; 11 AML cells (FIG. 39).

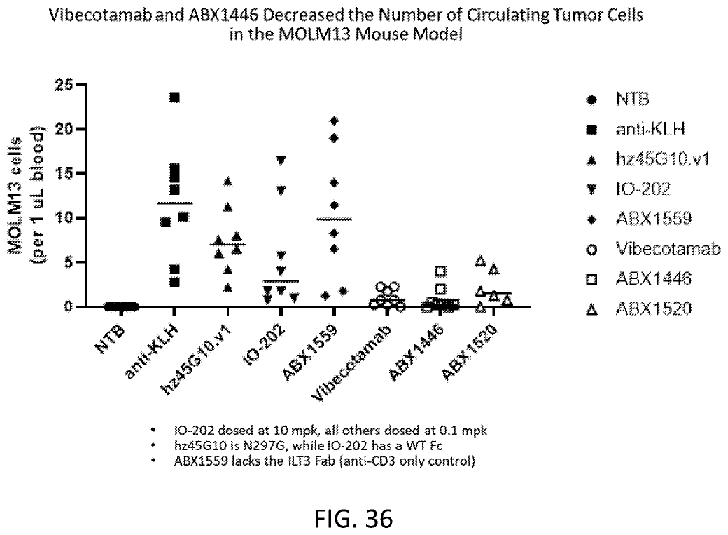

In the MOLM13 mouse model, ABX1446 and ABX1520 decreased the number of circulating tumor cells similarly to Vibecotamab. See FIG. 36 and Table 23.

TABLE 23

| Group | Avg. Tumor Cells Per 1 μL of Blood |
|---|---|
| NTB | 0 |
| anti-KLH | 11.7 |
| hz45G10 | 7.5 |
| IO-202 | 5.6 |
| ABX1559 | 10.4 |
| Vibecotamab | 1.0 |
| ABX1446 | 0.9 |
| ABX1520 | 2.2 |

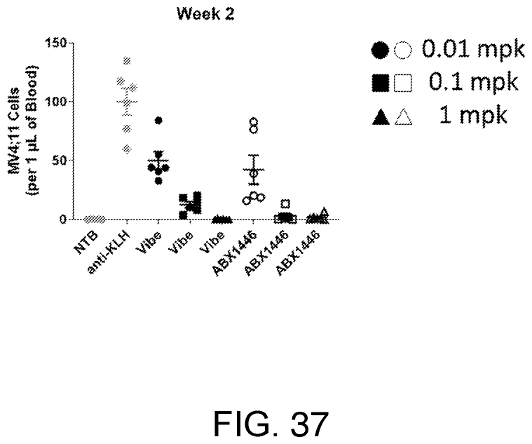

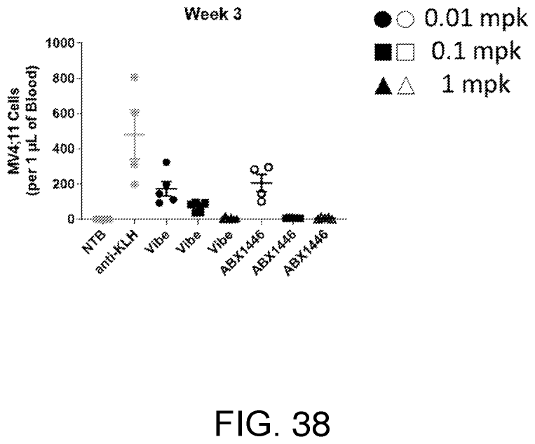

In the MV4; 11 mouse model, mice that received increasing concentrations (0.01 mpk, 0.1 mpk and 1 mpk) of ABX1446 had decreased numbers of circulating tumor cells at week 2 (FIG. 37) and week 3 (FIG. 38), similarly to Vibecotamab. See Table 24, which represents the number of MV4; 11 cells per μL of blood.

TABLE 24

| Group | Week 2* | Week 3* |
|---|---|---|
| NTB | 0.2 | .04 |
| anti-KLH | 100.1 | 480.4 |
| Vibecotamab 0.01 mpk | 50.2 | 173.4 |
| Vibecotamab 0.1 mpk | 12.5 | 71.0 |
| Vibecotamab 1 mpk | 0.4 | 6.1 |
| ABX1446 0.01 mpk | 42.4 | 206.6 |
| ABX1446 0.1 mpk | 3.2 | 6.9 |
| ABX1446 1 mpk | 1.9 | 6.6 |

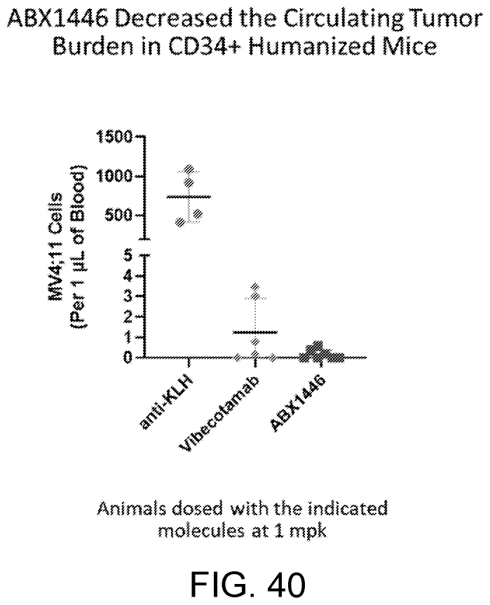

In the model with CD34⁺ humanized mice engrafted with MV4; 11 AML cells, ABX1446 decreased the number of circulating MV4; 11 cells per μL of blood. See FIG. 40 and Table 25, which represents the number of MV4; 11 cells per μL of blood. Mice received ABX1446, anti-KLH and Vibecotamab at 1 mpk.

TABLE 25

| Group | MV4; 11 Cells* |
|---|---|
| anti-KLH | 734.8 |
| Vibecotamab | 1.2 |
| ABX1446 | 0.2 |

The above described studies demonstrated that ABX1446 inhibited tumor growth in three different human AML mouse models.

### 7.4 Example 4: Characterization of ILT3×CD3 Bispecific Molecules in Primary Tumor Cell Culture

The activity of the ILT3×CD3 bispecific molecules was evaluated in the Native Tumor Microenvironment platform (Vivia Biotech). On this platform, whole bone marrow samples from human patients diagnosed with M5 AML were cultured with a dose titration of ABX1446. T cell activation and tumor cell depletion were evaluated. Briefly, whole bone marrow from three patients with M5 AML was evaluated in the Vivia Biotech Native Tumor Microenvironment platform. The demographics of the bone marrow donors is as follows:

TABLE 26
Patient Demographics

| Donor | FAB | | | |
|---|---|---|---|---|
| ID | Subtype | Age | Treatment Line | Prior Therapy |
| 13273 | M5 | 31 | Relapsed/Refractory | Idarubicin, |
| | | | | cytarabine |
| 15443 | M5 | 44 | Newly Diagnosed | Unknown |
| 15802 | M5b | 68 | Newly Diagnosed | Unknown |

For each sample, cryopreserved whole bone marrow was viably thawed and the ILT3 receptor density at baseline was quantified by flow cytometry using Quantibrite beads (BD Biosciences, 340495) and a PE-conjugated ILT3 antibody (clone ZM4.1, BD Biosciences, 333007). In addition, flow cytometry was used to count the numbers of tumor cells and T cells at baseline. The baseline effector:target (E:T) ratio was calculated for each sample.

TABLE 27
ILT3 Receptor Density and Baseline E:T Ratio

| | ILT3 Receptor Density | Baseline Effector:Target |
|---|---|---|
| Donor ID | (Antibody Binding Capacity) | (E:T) Ratio |
| 13273 | 23504 | 1:25 |
| 15443 | 7398 | 1:14 |
| 15802 | 5312 | 1:8 |

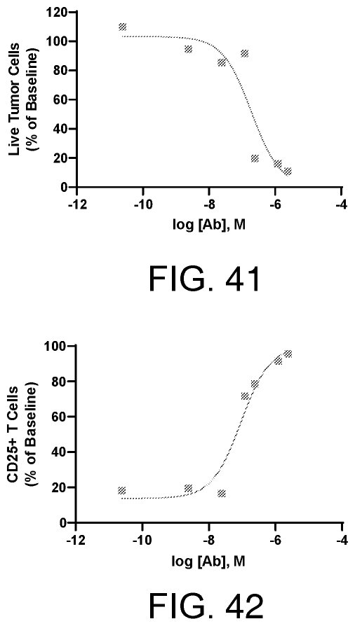

To evaluate the activity of ILT3×CD3 bispecific molecules, bone marrow samples were plated in serum-free media supplemented with fetal bovine serum (FBS) and a proprietary cocktail of growth factors. ABX1446 was added to the cultures in an 8-point dose titration (final concentration, 0.3-3× 10-6 mg/mL). Control conditions included bone marrow treated with PBS, and bone marrow treated with an isotype control antibody at 0.3 mg/mL. Cells were harvested at 72 and 120 hrs for evaluation of tumor cell depletion and T cell activation by flow cytometry. Results were normalized to the baseline values for each donor. ABX1446 induced dose-dependent tumor cell depletion and T cell activation in primary M5 AML bone marrow samples, as shown in FIG. 41 and FIG. 42 in one representative donor (out of 3 donors evaluated).

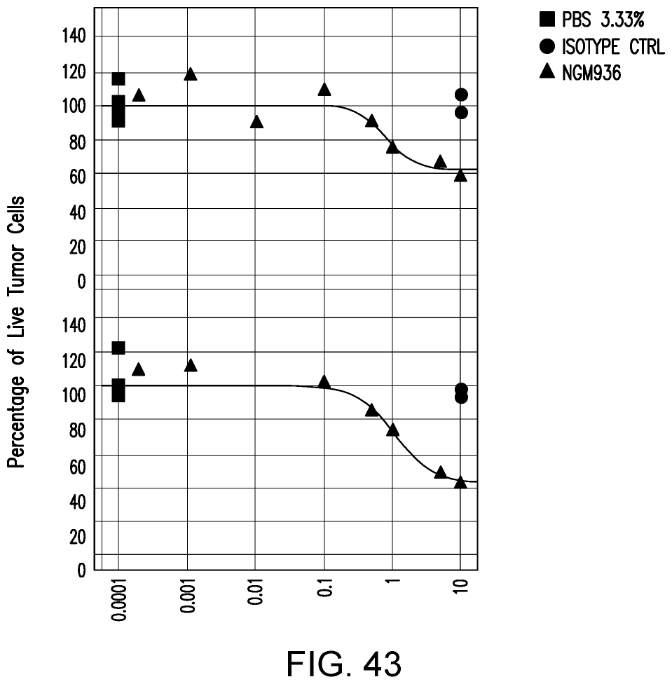

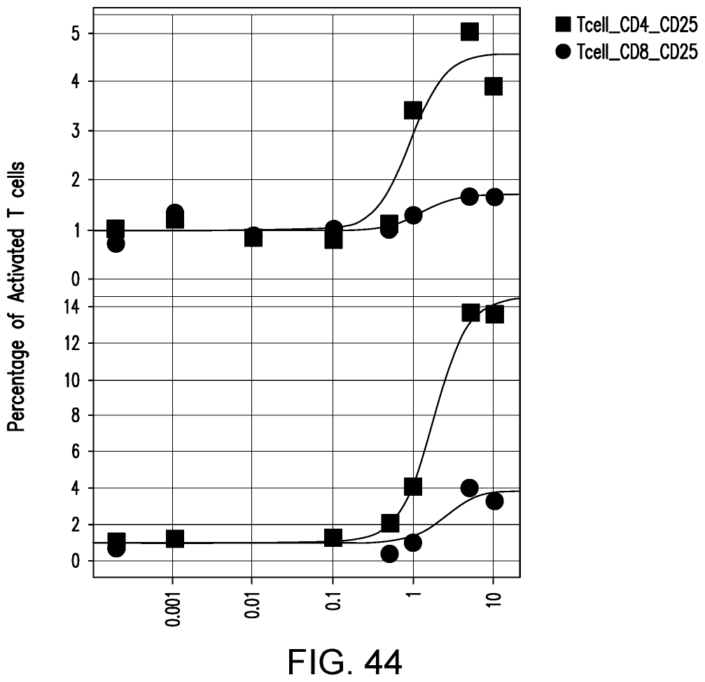

A similar study was initiated to evaluate the activity of ILT3×CD3 bispecific molecules in primary cultures of ILT3⁺ multiple myeloma samples. Fresh multiple myeloma bone marrow samples were first analyzed for ILT3 expression, and ILT3⁺ samples (defined as samples in which ≥60% of CD138⁺ myeloma blasts are ILT3⁺) with sufficient cell numbers and viability were evaluated in the Native Tumor Microenvironment platform. ABX1446 induced dose-dependent depletion of CD138⁺ multiple myeloma cells and concomitant T cell activation. Results from a representative donor are shown in FIG. 43 and FIG. 44.

SEQUENCES

| SEQ | | |
|---|---|---|
| ID | | |
| NO | Description | Sequence |
| 1 | Hz45G10 Heavy Chain | GFTFSDYGMH |
| | variable region CDR1 | |
| 2 | Hz45G10 Heavy Chain | YIFSGSSTIYYADTVKG |
| | variable region CDR2 | |
| 3 | Hz45G10 Heavy Chain | ADGRGAMDY |
| | variable region CDR3 | |
| 4 | Hz45G10 Light Chain | RASQDISKFLN |
| | variable region CDR1 | |
| 5 | Hz45G10 Light Chain | YTSRLHS |
| | variable region CDR2 | |
| 6 | Hz45G10 Light Chain | QQGNTLPWT |
| | variable region CDR3 | |
| 7 | Hz45G10 Heavy Chain | GFTFSDY |
| | variable region CDR1 | |
| 8 | Hz45G10 Heavy Chain | FSGSST |
| | variable region CDR2 | |
| 9 | Hz45G10 Heavy Chain | YIFSGSSTIY |
| | variable region CDR2 | |
| 10 | Hz45G10 Heavy Chain | DYGMH |
| | variable region CDR1 | |
| 11 | Hz45G10 Heavy Chain | SDYGMH |
| | variable region CDR1 | |
| 12 | Hz45G10 Heavy Chain | WVAYIFSGSSTIY |
| | variable region CDR2 | |
| 13 | Hz45G10 Heavy Chain | ARADGRGAMD |
| | variable region CDR3 | |
| 14 | Hz45G10 Light Chain | SKFLNWY |
| | variable region | |
| | CDR1 | |
| 15 | Hz45G10 Light Chain | LLIYYTSRLH |
| | variable region | |
| | CDR2 | |
| 16 | Hz45G10 Light Chain | QQGNTLPW |
| | variable region | |
| | CDR3 | |
| 17 | Hz45G10 Heavy chain | EVQLVESGGGLVQPGGSLRLSCAASGFTFSDYGMH |
| | variable region amino | WVRQAPGKGLEWVAYIFSGSSTIYYADTVKGRFTIS |
| | acid sequence (VH) | RDNAKNSLYLQMNSLRAEDTAVYYCARADGRGAM |
| | | DYWGQGTLVTVSS |
| 18 | Hz45G10 Light chain | DIQMTQSPSSLSASVGDRVTITCRASQDISKFLNWYQ |
| | variable region amino | QKPGKAPKLLIYYTSRLHSGVPSRFSGSGSGTDFTFTI |
| | acid sequence (VL) | SSLQPEDIATYFCQQGNTLPWTFGGGTKLEIK |
| 19 | Hz45G10 Heavy chain | EVQLVESGGGLVQPGGSLRLSCAASGFTFSDYGMH |
| | amino acid sequence | WVRQAPGKGLEWVAYIFSGSSTIYYADTVKGRFTIS |
| | | RDNAKNSLYLQMNSLRAEDTAVYYCARADGRGAM |
| | | DYWGQGTLVTVSSASTKGPSVFPLAPSSKSTSGGTA |
| | | ALGCLVKDYFPEPVTVSWNSGALTSGVHTFPAVLQS |
| | | SGLYSLSSVVTVPSSSLGTQTYICNVNHKPSNTKVDK |
| | | KVEPKSCDKTHTCPPCPAPEAAGGPSVFLFPPKPKDT |
| | | LMISRTPEVTCVVVDVSHEDPEVKFNWYVDGVEVH |
| | | NAKTKPREEQYNSTYRVVSVLTVLHQDWLNGKEYK |
| | | CKVSNKALPAPIEKTISKAKGQPREPQVYTLPPSREE |
| | | MTKNQVSLWCLVKGFYPSDIAVEWESNGQPENNYK |
| | | TTPPVLDSDGSFFLYSKLTVDKSRWQQGNVFSCSVM |
| | | HEALHNHYTQKSLSLSPGK |
| 20 | Hz45G10 Light chain | DIQMTQSPSSLSASVGDRVTITCRASQDISKFLNWYQ |
| | amino acid sequence | QKPGKAPKLLIYYTSRLHSGVPSRFSGSGSGTDFTFTI |
| | | SSLQPEDIATYFCQQGNTLPWTFGGGTKLEIKRTVA |
| | | APSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC |
| 21 | 3A3 Heavy chain | GFSLTSYGVH |
| | variable region CDR1 | |
| 22 | 3A3 Heavy chain | VIWPGGTINYNSALMS |
| | variable region CDR2 | |
| 23 | 3A3 Heavy chain | DKYDGGWFAY |
| | variable region CDR3 | |
| 24 | 3A3 Light chain | KASQNVRTAVA |
| | variable region CDR1 | |
| 25 | 3A3 Light chain | LASNRHT |
| | variable region CDR2 | |
| 26 | 3A3 Light chain | LQHLNYPLT |
| | variable region CDR3 | |
| 27 | 3A3 Heavy chain | GFSLTSY |
| | variable region CDR1 | |
| 28 | 3A3 Heavy chain | WPGGT |
| | variable region CDR2 | |
| 29 | 3A3 Heavy chain | VIWPGGTIN |
| | variable region CDR2 | |
| 30 | 3A3 Heavy chain | SYGVH |
| | variable region CDR1 | |
| 31 | 3 A3 Heavy chain | TSYGVH |
| | variable region CDR1 | |
| 32 | 3A3 Heavy chain | WLGVIWPGGTIN |
| | variable region CDR2 | |
| 33 | 3A3 Heavy chain | ASDKYDGGWFA |
| | variable region CDR3 | |
| 34 | 3A3 Light chain | RTAVAWY |
| | variable region CDR1 | |
| 35 | 3A3 Light chain | ALIYLASNRH |
| | variable region CDR2 | |
| 36 | 3A3 Light chain | LQHLNYPL |
| | variable region CDR3 | |
| 37 | 3A3 Heavy chain | QVQLKESGPGLVAPSQSLSITCTVSGFSLTSYGVHW |
| | variable region amino | VRQPPGKGLEWLGVIWPGGTINYNSALMSRLSISKD |
| | acid sequence | NSKSQVFLKLNSLQTDDTAMYYCASDKYDGGWFA |
| | | YWGQGTLVTVSA |
| 38 | 3A3 Light chain | DIVMTQSQKFMSTSVGDRVSITCKASQNVRTAVAW |
| | variable region amino | YQQKPGQSPEALIYLASNRHTGVPDRFTGSGSGTDFS |
| | acid sequence | LSISNVQSEDLADYFCLQHLNYPLTFGSGTKLEIK |
| 39 | 5A7 Heavy chain | GFTFSSYGMS |
| | variable region CDR1 | |
| 40 | 5A7 Heavy chain | TISGGGSYTNYPDSVKG |
| | variable region CDR2 | |
| 41 | 5A7 Heavy chain | REWRMTLYAMDY |
| | variable region CDR3 | |
| 42 | 5A7 Light chain | RASESVDSYGNSFMH |
| | variable region CDR1 | |
| 43 | 5A7 Light chain | LTSNLES |
| | variable region CDR2 | |
| 44 | 5A7 Light chain | QQNNEDPFT |
| | variable region CDR3 | |
| 45 | 5A7 Heavy chain | GFTFSSY |
| | variable region CDR1 | |
| 46 | 5A7 Heavy chain | SGGGSY |
| | variable region CDR2 | |
| 47 | 5A7 Heavy chain | TISGGGSYTN |
| | variable region CDR2 | |
| 48 | 5A7 Heavy chain | SYGMS |
| | variable region CDR1 | |
| 49 | 5A7 Heavy chain | SSYGMS |
| | variable region CDR1 | |
| 50 | 5A7 Heavy chain | WVATISGGGSYTN |
| | variable region CDR2 | |
| 51 | 5A7 Heavy chain | ARREWRMTLYAMD |
| | variable region CDR3 | |
| 52 | 5A7 Light chain | DSYGNSFMHWY |
| | variable region CDR1 | |
| 53 | 5A7 Light chain | LLIYLTSNLE |
| | variable region CDR2 | |
| 54 | 5A7 Light chain | QQNNEDPF |
| | variable region CDR3 | |
| 55 | 5A7 Heavy chain | EVKLVESGGGLVKPGGSLKLSCAASGFTFSSYGMSW |
| | variable region amino | VRQTPEKRLEWVATISGGGSYTNYPDSVKGRLTISR |
| | acid sequence | DNAKKNLYLEMSSLRSEDTALYYCARREWRMTLY |
| | | AMDYWGQGTSVTVSS |
| 56 | 5A7 Light chain | NIVLTQSPASLAVSLGQRATISCRASESVDSYGNSFM |
| | variable region amino | HWYQQKPGQAPKLLIYLTSNLESGVPARFSGSGSRT |
| | acid sequence | DFTLTIDPVEADDAATYYCQQNNEDPFTFGSGTKLEI |
| | | K |
| 57 | 12A12 Heavy chain | GYTFTDYNMD |
| | variable region CDR1 | |
| 58 | 12A12 Heavy chain | YIYPNNGGTGYNQKENS |
| | variable region CDR2 | |
| 59 | 12A12 Heavy chain | SPYYDYVGSYAMDY |
| | variable region CDR3 | |
| 60 | 12A12 Light chain | TASSSVSSSYLH |
| | variable region CDR1 | |
| 61 | 12A12 Light chain | STSNLAS |
| | variable region CDR2 | |
| 62 | 12A12 Light chain | HQYHRSPRT |
| | variable region CDR3 | |
| 63 | 12A12 Heavy chain | GYTFTDY |
| | variable region CDR1 | |
| 64 | 12A12 Heavy chain | YPNNGG |
| | variable region CDR2 | |
| 65 | 12A12 Heavy chain | YIYPNNGGTG |
| | variable region CDR2 | |
| 66 | 12A12 Heavy chain | DYNMD |
| | variable region CDR1 | |
| 67 | 12A12 Heavy chain | TDYNMD |
| | variable region CDR1 | |
| 68 | 12A12 Heavy chain | WIGYIYPNNGGTG |
| | variable region CDR2 | |
| 69 | 12A12 Heavy chain | ASSPYYDYVGSYAMD |
| | variable region CDR3 | |
| 70 | 12A12 Light chain | SSSYLHWY |
| | variable region CDR1 | |
| 71 | 12A12 Light chain | LWIYSTSNLA |
| | variable region CDR2 | |
| 72 | 12A12 Light chain | HQYHRSPR |
| | variable region CDR3 | |
| 73 | 12A12 Heavy chain | EVQLQQSGPELVKPGASVKISCKASGYTFTDYNMD |
| | variable region amino | WVKQSHGKSLEWIGYIYPNNGGTGYNQKFNSKATL |
| | acid sequence | TVDKSSSTAYMELHSLTSEDSAVYYCASSPYYDYVG |
| | | SYAMDYWGQGTSVTVSS |
| 74 | 12A12 Light chain | QIVLTQSPAIMSASLGERVTMTCTASSSVSSSYLHWY |
| | variable region amino | QQKPGSSPKLWIYSTSNLASGVPARFSGSGSGTSYSL |
| | acid sequence | TISSMEAEDAATYYCHQYHRSPRTFGGGTKLEIK |
| 75 | 16C5 Heavy chain | GYTFTDYNMD |
| | variable region CDR1 | |
| 76 | 16C5 Heavy chain | YIYPSNGGTGYNQKFKS |
| | variable region CDR2 | |
| 77 | 16C5 Heavy chain | VPYYDYLYYYAMDY |
| | variable region CDR3 | |
| 78 | 16C5 Light chain | RASSSVSFMH |
| | variable region CDR1 | |
| 79 | 16C5 Light chain | ATSNLAS |
| | variable region CDR2 | |
| 80 | 16C5 Light chain | QQWSTNPYMYT |
| | variable region CDR3 | |
| 81 | 16C5 Heavy chain | GYTFTDY |
| | variable region CDR1 | |
| 82 | 16C5 Heavy chain | YPSNGG |
| | variable region CDR2 | |
| 83 | 16C5 Heavy chain | YIYPSNGGTG |
| | variable region CDR2 | |
| 84 | 16C5 Heavy chain | DYNMD |
| | variable region CDR1 | |
| 85 | 16C5 Heavy chain | TDYNMD |
| | variable region CDR1 | |
| 86 | 16C5 Heavy chain | WIGYIYPSNGGTG |
| | variable region CDR2 | |
| 87 | 16C5 Heavy chain | ARVPYYDYLYYYAMD |
| | variable region CDR3 | |
| 88 | 16C5 Light chain | SFMHWY |
| | variable region CDR1 | |
| 89 | 16C5 Light chain | PWIYATSNLA |
| | variable region CDR2 | |
| 90 | 16C5 Light chain | QQWSTNPYMY |
| | variable region CDR3 | |
| 91 | 16C5 Heavy chain | EVQLQQSGPELVKPGASVKISCKASGYTFTDYNMD |
| | variable region amino | WVKQSHGKSLEWIGYIYPSNGGTGYNQKFKSKATL |
| | acid sequence | TVDKSSNTAYMELHSLTSEDSAVYYCARVPYYDYL |
| | | YYYAMDYWGQGTSVTVSS |
| 92 | 16C5 Light chain | QIVLSQSPAILSASPGEKVTMACRASSSVSFMHWYQ |
| | variable region amino | QKPGSSPQPWIYATSNLASGVPARFSGSGSGTSYSLT |
| | acid sequence | ISRVEAEDAATYYCQQWSTNPYMYTFGGGTKLEIK |
| 93 | 48A6 Heavy chain | GFTFSSYGMS |
| | variable region CDR1 | |
| 94 | 48A6 Heavy chain | TISSGGTYTFYPDSVKG |
| | variable region CDR2 | |
| 95 | 48A6 Heavy chain | RGWLLHYYAMDY |
| | variable region CDR3 | |
| 96 | 48A6 Light chain | RPSESVDSFGNSFMH |
| | variable region CDR1 | |
| 97 | 48A6 Light chain | LSSKLES |
| | variable region CDR2 | |
| 98 | 48A6 Light chain | QQHNEDPFT |
| | variable region CDR3 | |
| 99 | 48A6 Heavy chain | GFTFSSY |
| | variable region CDR1 | |
| 100 | 48A6 Heavy chain | SSGGTY |
| | variable region CDR2 | |
| 101 | 48A6 Heavy chain | TISSGGTYTF |
| | variable region CDR2 | |
| 102 | 48A6 Heavy chain | SYGMS |
| | variable region CDR1 | |
| 103 | 48A6 Heavy chain | SSYGMS |
| | variable region CDR1 | |
| 104 | 48A6 Heavy chain | WVATISSGGTYTF |
| | variable region CDR2 | |
| 105 | 48A6 Heavy chain | ARRGWLLHYYAMD |
| | variable region CDR3 | |
| 106 | 48A6 Light chain | DSFGNSFMHWF |
| | variable region CDR1 | |
| 107 | 48A6 Light chain | LLIYLSSKLE |
| | variable region CDR2 | |
| 108 | 48A6 Light chain | QQHNEDPF |
| | variable region CDR3 | |
| 109 | 48A6 Heavy chain | EVQLVESGGDLMKPGGSLKLSCAASGFTFSSYGMS |
| | variable region amino | WVRQTPDKRLEWVATISSGGTYTFYPDSVKGRFTIS |
| | acid sequence | RDNAKNTLYLQMSSLKSEDTAMYYCARRGWLLHY |
| | | YAMDYWGQGTSVTVSS |
| 110 | 48A6 Light chain | NIVLTQSPASLAVSLGQRATISCRPSESVDSFGNSFM |
| | variable region amino | HWFQQKPGQPPKLLIYLSSKLESGVPARFSGSGSRTD |
| | acid sequence | FTLTIDPVEADDAATYYCQQHNEDPFTFGSGTKLEIK |
| 111 | 53F10 Heavy chain | GFTFSDYGMH |
| | variable region CDR1 | |
| 112 | 53F10 Heavy chain | YISTGIITVYYADTVKG |
| | variable region CDR2 | |
| 113 | 53F10 Heavy chain | ADGRGAMDY |
| | variable region CDR3 | |
| 114 | 53F10 Light chain | RASQDISNFLN |
| | variable region CDR1 | |
| 115 | 53F10 Light chain | YTSRLHS |
| | variable region CDR2 | |
| 116 | 53F10 Light chain | QQGNTLPWT |
| | variable region CDR3 | |
| 117 | 53F10 Heavy chain | GFTFSDY |
| | variable region CDR1 | |
| 118 | 53F10 Heavy chain | STGIIT |
| | variable region CDR2 | |
| 119 | 53F10 Heavy chain | YISTGIITVY |
| | variable region CDR2 | |
| 120 | 53F10 Heavy chain | DYGMH |
| | variable region CDR1 | |
| 121 | 53F10 Heavy chain | SDYGMH |
| | variable region CDR1 | |
| 122 | 53F10 Heavy chain | WVAYISTGIITVY |
| | variable region CDR2 | |
| 123 | 53F10 Heavy chain | ARADGRGAMD |
| | variable region CDR3 | |
| 124 | 53F10 Light chain | SNFLNWY |
| | variable region CDR1 | |
| 125 | 53F10 Light chain | LLIYYTSRLH |
| | variable region CDR2 | |
| 126 | 53F10 Light chain | QQGNTLPW |
| | variable region CDR3 | |
| 127 | 53F10 Heavy chain | EVQVVESGGGLVKPGGSLKLSCAASGFTFSDYGMH |
| | variable region amino | WVRQAPEKGLEWVAYISTGIITVYYADTVKGRFTMS |
| | acid sequence | RDNAKNTLFLQMTSLRSEDTAIYYCARADGRGAMD |
| | | YWGQGTSVIVSS |
| 128 | 53F10 Light chain | DIQMTQTTSSLSASLGDRVTISCRASQDISNFLNWYQ |
| | variable region amino | QKPDGTVTLLIYYTSRLHSGVPSRFSGSGSGTDYSLTI |
| | acid sequence | SNLEQEDFATYFCQQGNTLPWTFGGGTKLEIK |
| 129 | Hz5A7.v5 Heavy chain | GFTFSSYGMS |
| | variable region CDR1 | |
| 130 | Hz5A7.v5 Heavy chain | TISGGGSYTNYPDSVKG |
| | variable region CDR2 | |
| 131 | Hz5A7.v5 Heavy chain | REWRYTLYAMDY |
| | variable region CDR3 | |
| 132 | Hz5A7.v5 Light chain | RASESVESYGSSFMH |
| | variable region CDR1 | |
| 133 | Hz5A7.v5 Light chain | LTSNLES |
| | variable region CDR2 | |
| 134 | Hz5A7.v5 Light chain | QQNNEDPFT |
| | variable region CDR3 | |
| 135 | Hz5A7.v5 Heavy chain | GFTFSSY |
| | variable region CDR1 | |
| 136 | Hz5A7.v5 Heavy chain | SGGGSY |
| | variable region CDR2 | |
| 137 | Hz5A7.v5 Heavy chain | TISGGGSYTN |
| | variable region CDR2 | |
| 138 | Hz5A7.v5 Heavy chain | SYGMS |
| | variable region CDR1 | |
| 139 | Hz5A7.v5 Heavy chain | SSYGMS |
| | variable region CDR1 | |
| 140 | Hz5A7.v5 Heavy chain | WVATISGGGSYTN |
| | variable region CDR2 | |
| 141 | Hz5A7.v5 Heavy chain | ARREWRYTLYAMD |
| | variable region CDR3 | |
| 142 | Hz5A7.v5 Light chain | ESYGSSFMHWY |
| | variable region CDR1 | |
| 143 | Hz5A7.v5 Light chain | LLIYLTSNLE |
| | variable region CDR2 | |
| 144 | Hz5A7.v5 Light chain | QQNNEDPF |
| | variable region CDR3 | |
| 145 | Hz5A7.v5 Heavy chain | EVQLVESGGGLVQPGGSLRLSCAASGFTFSSYGMSW |
| | variable region amino | VRQAPGKGLEWVATISGGGSYTNYPDSVKGRFTISR |
| | acid sequence | DNAKNSLYLQMNSLRAEDTAVYYCARREWRYTLYA |
| | | MDYWGQGTTVTVSS |
| 146 | Hz5A7.v5 Light chain | DIQLTQSPSFLSASVGDRVTITCRASESVESYGSSFMH |
| | variable region amino | WYQQKPGKAPKLLIYLTSNLESGVPSRFSGSGSGTEF |
| | acid sequence | TLTISSLQPEDFATYYCQQNNEDPFTFGQGTKLEIK |
| 147 | CD3 scFv-Fc amino | EVQLLESGGGLVQPGGSLKLSCAASGFTFNTYAMN |
| | acid sequence | WVRQAPGKCLEWVARIRSKYNNYATYYADSVKDR |
| | | FTISRDDSKNTAYLQMNNLKTEDTAVYYCVRHGNF |
| | | GNSYVSWFAYWGQGTLVTVSSGGGGSGGGGSGGG |
| | | GSELVVTQEPSLTVSPGGTVTLTCRSSTGAVTTSNYA |
| | | NWVQQKPGQAPRGLIGGTNKRAPGTPARFSGSLLG |
| | | GKAALTLSGVQPEDEAEYYCALWYSNLWVFGCGT |
| | | KLTVLGGGGSDKTHTCPPCPAPEAAGGPSVFLFPPKP |
| | | KDTLMISRTPEVTCVVVDVSHEDPEVKFNWYVDGV |
| | | EVHNAKTKPREEQYNSTYRVVSVLTVLHQDWLNGK |
| | | EYKCKVSNKALPAPIEKTISKAKGQPREPQVYTLPPS |
| | | REEMTKNQVSLSCAVKGFYPSDIAVEWESNGQPEN |
| | | NYKTTPPVLDSDGSFFLVSKLTVDKSRWQQGNVFSC |
| | | SVMHEALHNHYTQKSLSLSPGK |
| 148 | CD3 scFv-Fc nucleic | GAGGTGCAGCTGTTGGAATCTGGCGGAGGATTGG |
| | acid sequence | TTCAGCCTGGCGGCTCTCTGAAGCTGTCTTGTGCC |
| | | GCTTCTGGCTTCACCTTCAACACCTACGCCATGAA |
| | | CTGGGTCCGACAGGCCCCTGGCAAATGCCTGGAA |
| | | TGGGTCGCCCGGATCAGATCCAAGTACAACAATT |
| | | ACGCCACCTACTACGCCGACTCCGTGAAGGACCG |
| | | GTTCACCATCTCTCGGGACGACTCCAAGAACACCG |
| | | CCTACCTGCAGATGAACAACCTCAAGACCGAGGA |
| | | TACCGCCGTGTACTACTGTGTGCGGCACGGCAACT |
| | | TCGGCAACTCCTATGTGTCTTGGTTTGCCTACTGG |
| | | GGCCAGGGCACACTGGTCACAGTTTCTAGCGGCG |
| | | GAGGTGGAAGCGGAGGCGGAGGTAGTGGTGGTGG |
| | | CGGATCTGAACTGGTGGTCACCCAAGAGCCTAGC |
| | | CTGACAGTTTCTCCTGGCGGCACCGTGACACTGAC |
| | | CTGTAGATCTTCTACCGGCGCTGTGACCACCTCCA |
| | | ACTACGCCAATTGGGTGCAGCAGAAGCCAGGCCA |
| | | GGCTCCTAGAGGACTGATCGGCGGCACAAACAAG |
| | | AGAGCCCCTGGAACTCCTGCCAGGTTCTCTGGATC |
| | | TCTGCTCGGCGGAAAGGCTGCTCTGACACTGTCTG |
| | | GTGTCCAGCCTGAGGACGAGGCCGAGTATTACTG |
| | | TGCCCTGTGGTACTCCAACCTGTGGGTGTTCGGCT |
| | | GTGGCACCAAGCTGACAGTTCTCGGAGGCGGCGG |
| | | ATCCGACAAGACCCATACTTGTCCTCCATGTCCTG |
| | | CTCCAGAGGCTGCTGGTGGCCCTTCCGTGTTTCTG |
| | | TTCCCTCCAAAGCCTAAGGACACCCTGATGATCTC |
| | | TCGGACCCCTGAAGTGACCTGCGTGGTGGTCGATG |
| | | TGTCTCACGAGGACCCAGAAGTGAAGTTCAATTG |
| | | GTACGTGGACGGCGTGGAAGTGCATAACGCCAAG |
| | | ACCAAGCCTAGAGAGGAACAGTACAACTCCACCT |
| | | ACAGAGTGGTGTCCGTGCTGACCGTGCTGCACCA |
| | | GGATTGGCTGAACGGCAAAGAGTACAAGTGCAAG |
| | | GTGTCCAACAAGGCCCTGCCTGCTCCTATCGAAAA |
| | | GACCATCTCCAAGGCCAAGGGCCAGCCTAGGGAA |
| | | CCCCAGGTTTACACCTTGCCTCCAAGCCGGGAAGA |
| | | GATGACCAAGAACCAGGTGTCCCTGTCCTGTGCCG |
| | | TGAAGGGCTTCTACCCTTCCGATATCGCCGTGGAA |
| | | TGGGAGAGCAATGGCCAGCCAGAGAACAACTACA |
| | | AGACAACCCCTCCTGTGCTGGACTCCGACGGCTCA |
| | | TTCTTCCTGGTGTCTAAGCTGACTGTGGACAAGTC |
| | | CAGATGGCAGCAGGGCAACGTGTTCTCCTGCTCCG |
| | | TGATGCACGAGGCCCTGCACAATCACTACACACA |
| | | GAAGTCCCTGAGCCTGTCTCCTGGCAAG |
| 149 | CD3 VH amino acid | EVQLLESGGGLVQPGGSLKLSCAASGFTFNTYAMN |
| | sequence | WVRQAPGKCLEWVARIRSKYNNYATYYADSVKDR |
| | | FTISRDDSKNTAYLQMNNLKTEDTAVYYCVRHGNF |
| | | GNSYVSWFAYWGQGTLVTVSS |
| 150 | CD3 VL amino acid | ELVVTQEPSLTVSPGGTVTLTCRSSTGAVTTSNYAN |
| | sequence | WVQQKPGQAPRGLIGGTNKRAPGTPARFSGSLLGG |
| | | KAALTLSGVQPEDEAEYYCALWYSNLWVFGCGTKL |
| | | TVL |
| 151 | CD3 scFv amino acid | EVQLLESGGGLVQPGGSLKLSCAASGFTFNTYAMN |
| | sequence | WVRQAPGKCLEWVARIRSKYNNYATYYADSVKDR |
| | | FTISRDDSKNTAYLQMNNLKTEDTAVYYCVRHGNF |
| | | GNSYVSWFAYWGQGTLVTVSSGGGGSGGGGSGGG |
| | | GSELVVTQEPSLTVSPGGTVTLTCRSSTGAVTTSNYA |
| | | NWVQQKPGQAPRGLIGGTNKRAPGTPARFSGSLLG |
| | | GKAALTLSGVQPEDEAEYYCALWYSNLWVFGCGT |
| | | KLTVL |
| 152 | CD3 Heavy Chain | GFTFNTYAMN |
| | variable region CDR1 | |
| 153 | CD3 Heavy Chain | RIRSKYNNYATYYADSVKD |
| | variable region CDR2 | |
| 154 | CD3 Heavy Chain | HGNFGNSYVSWFAY |
| | variable region CDR3 | |
| 155 | CD3 Light Chain | RSSTGAVTTSNYAN |
| | variable region CDR1 | |
| 156 | CD3 Light Chain | GTNKRAP |
| | variable region CDR2 | |
| 157 | CD3 Light Chain | ALWYSNLWV |
| | variable region CDR3 | |
| 158 | CD3 VH amino acid | EVQLVESGGGLVQPGGSLKLSCAASGFEFNKYAMN |
| | sequence | WVRQAPGKGLEWVARIRSKYNNYETYYADSVKDR |
| | | FTISRDDSKNTAYLQMNNLKTEDTAVYYCVRHGNF |
| | | GNSLISYWAYWGQGTLVTVSS |
| 159 | CD3 VL amino acid | QTVVTQEPSLTVSPGGTVTLTCGSSSGAVTSGNYPN |
| | sequence | WVQQKPGQAPRGLIGGTKFGAPGTPARFSGSLLGGK |
| | | AALTLSGVQPEDEAEYYCVLWYSNRWVFGGGTKLT |
| | | VL |
| 160 | CD3 scFv amino acid | EVQLVESGGGLVQPGGSLKLSCAASGFEFNKYAMN |
| | sequence | WVRQAPGKGLEWVARIRSKYNNYETYYADSVKDR |
| | | FTISRDDSKNTAYLQMNNLKTEDTAVYYCVRHGNF |
| | | GNSLISYWAYWGQGTLVTVSSGGGGSGGGGSGGGG |
| | | SQTVVTQEPSLTVSPGGTVTLTCGSSSGAVTSGNYPN |
| | | WVQQKPGQAPRGLIGGTKFGAPGTPARFSGSLLGGK |
| | | AALTLSGVQPEDEAEYYCVLWYSNRWVFGGGTKLT |
| | | VL |
| 161 | CD3 Heavy Chain | GFEFNKYAMN |
| | variable region CDR1 | |
| 162 | CD3 Heavy Chain | RIRSKYNNYETYYADSVKD |
| | variable region CDR2 | |
| 163 | CD3 Heavy Chain | HGNFGNSLISYWAY |
| | variable region CDR3 | |
| 164 | CD3 Light Chain | GSSSGAVTSGNYPN |
| | variable region CDR1 | |
| 165 | CD3 Light Chain | GTKFGAP |
| | variable region CDR2 | |
| 166 | CD3 Light Chain | VLWYSNRWV |
| | variable region CDR3 | |
| 167 | Hz45G10 Heavy chain | gaggtgcagctggttgaatctggcggaggactggttcagcctggcggatctctgag |
| | nucleotide sequence | actgtcttgtgccgccagcggcttcaccttcagcgattatggcatgcactgggtccga |
| | | caggcccctggcaaaggacttgagtgggtcgcctacatcttcagcggcagcagca |
| | | ccatctactacgccgacacagtgaagggcagattcaccatcagccgggacaacgc |
| | | caagaacagcctgtacctgcagatgaactccctgagagccgaggacaccgccgtg |
| | | tactattgtgccagagccgatggcagaggcgctatggattattggggccagggcac |
| | | cctggtcaccgtttctagcgctagtaccaagggacccagcgtgttccctctggctcct |
| | | agcagcaagtctacaagcggaggaacagccgctctgggctgcctggtcaaggatt |
| | | actttcccgagcctgtgaccgtgtcctggaatagcggagcactgacaagcggcgtg |
| | | cacacctttccagctgtgctgcaaagcagcggcctgtactctctgagcagcgtggtc |
| | | acagtgccaagctctagcctgggcacccagacctacatctgcaatgtgaaccacaa |
| | | gcctagcaacaccaaggtggacaagaaggtggaacccaagagctgcgacaagac |
| | | ccacacctgtcctccatgtcctgctccagaagctgctggcggcccttccgtgtttctgt |
| | | tccctccaaagcctaaggacaccctgatgatcagcagaacccctgaagtgacctgc |
| | | gtggtggtggatgtgtctcacgaggaccccgaagtgaagttcaattggtacgtggac |
| | | ggcgtggaagtgcacaacgccaagaccaagcctagagaggaacagtacaacagc |
| | | acctacagagtggtgtccgtgctgaccgtgctgcaccaggattggctgaacggcaa |
| | | agagtacaagtgcaaggtgtccaacaaggccctgcctgctcctatcgagaaaacca |
| | | tcagcaaggccaagggccagcctagggaaccccaggtttacacactgcctccaag |
| | | ccgggaagagatgaccaagaaccaggtgtccctgtggtgcctcgtgaagggcttct |
| | | acccttccgatatcgccgtggaatgggagagcaatggccagcctgagaacaactac |
| | | aagacaacccctcctgtgctggacagcgacggctcattcttcctgtacagcaagctg |
| | | acagtggacaagtccagatggcagcagggcaacgtgttcagctgcagcgtgatgc |
| | | acgaggccctgcacaaccactacacccagaagtccctgagcctgtctcctggcaaa |
| 168 | Hz45G10 Light chain | gacatccagatgacccagtctccatcctctctgtccgcctctgtgggcgacagagtg |
| | nucleotide sequence | accatcacctgtagagccagccaggatatctccaagttcctgaactggtatcagcag |
| | | aagcccggcaaggcccctaagctgctgatctactacacctctcggctgcactctggc |
| | | gtgccctctagattttctggctccggctctggcaccgactttacctttacaatctccagc |
| | | ctgcagcctgaggatatcgctacctacttctgccagcaaggcaacaccctgccttgg |
| | | acatttggcggaggcaccaagctggaaatcaagcgtacggtggctgcaccatctgt |
| | | cttcatcttcccgccatctgatgagcagttgaaatctggaactgcctctgttgtgtgcct |
| | | gctgaataacttctatcccagagaggccaaagtacagtggaaggtggataacgccc |
| | | tccaatcgggtaactcccaggagagtgtcacagagcaggacagcaaggacagca |
| | | cctacagcctcagcagcaccctgacgctgagcaaagcagactacgagaaacacaa |
| | | agtctacgcctgcgaagtcacccatcagggcctgagctcgcccgtcacaaagagct |
| | | tcaacaggggagagtgt |
| 169 | Heavy chain of anti- | EVQLVESGGGLVQPGGSLRLSCAASGFTFSDYGMHW |
| | ILT3 binding region of | VRQAPGKGLEWVAYIFSGSSTIYYADTVKGRFTISRD |
| | F13 | NAKNSLYLQMNSLRAEDTAVYYCARADGRGAMDY |
| | | WGQGTLVTVSSASTKGPSVFPLAPSSKSTSGGTAALG |
| | | CLVKDYFPEPVTVSWNSGALTSGVHTFPAVLQSSGL |
| | | YSLSSVVTVPSSSLGTQTYICNVNHKPSNTKVDKKVE |
| | | PKSCGGGGSEVQLVESGGGLVQPGGSLRLSCAASGF |
| | | TFSDYGMHWVRQAPGKGLEWVAYIFSGSSTIYYADT |
| | | VKGRFTISRDNAKNSLYLQMNSLRAEDTAVYYCARA |
| | | DGRGAMDYWGQGTLVTVSSASTKGPSVFPLAPSSKS |
| | | TSGGTAALGCLVKDYFPEPVTVSWNSGALTSGVHTF |
| | | PAVLQSSGLYSLSSVVTVPSSSLGTQTYICNVNHKPS |
| | | NTKVDKKVEPKSCDKTHTCPPCPAPEAAGGPSVFLFP |
| | | PKPKDTLMISRTPEVTCVVVDVSHEDPEVKFNWYVD |
| | | GVEVHNAKTKPREEQYNSTYRVVSVLTVLHQDWLN |
| | | GKEYKCKVSNKALPAPIEKTISKAKGQPREPQVYTLP |
| | | PSREEMTKNQVSLWCLVKGFYPSDIAVEWESNGQPE |
| | | NNYKTTPPVLDSDGSFFLYSKLTVDKSRWQQGNVFS |
| | | CSVMHEALHNHYTQKSLSLSPGK |

What is claimed is:

1. A binding agent comprising a first binding region that binds to human ILT3 and a second binding region that binds to human CD3, wherein the CD3 binding region comprises an anti-CD3 scFv.

2. The binding agent of claim 1, wherein the first binding region comprises an anti-ILT3 Fab.

3. The binding agent of claim 1 or 2, wherein the binding affinity of the first binding region for human ILT3 is higher than the binding affinity of the second binding region for human CD3.

4. The binding agent of claim 3, the binding affinity of the first binding region for human ILT3 is between about 10 folds and about 100 folds higher than the binding affinity of the second binding region for human CD3.

5. The binding agent of any one of claims 1-4, further comprises a Fc region.

6. The binding agent of claim 5, comprising:
(i) a first polypeptide comprising the anti-CD3 scFv, a first CH2 domain, and a first CH3 domain;
(ii) a second polypeptide comprising a VH domain of the first binding region, a CH1 domain, a second CH2 domain, and a second CH3 domain; and
(iii) a third polypeptide comprising a VL domain of the first binding region and a CL domain,
wherein the VH domain of the first binding region, the CH1 domain, the VL domain of the first binding region, and the CL domain form the anti-ILT3 Fab, and the first CH2 domain, the second CH2 domain, the first CH3 domain, and the second CH3 domain form the Fc region.

7. The binding agent of claim 6, wherein the first polypeptide comprises one or more amino acid mutations that form an engineered cavity, and the second polypeptide comprising one or more amino acid mutations that form an engineered protuberance, and wherein the first polypeptide dimerizes with the second polypeptide via positioning of the protuberance into the cavity.

8. The binding agent of any of claims 1-7, wherein the second binding region comprises a VH domain comprising a HCDR1, a HCDR2, and a HCDR3 of the amino acid sequence set forth in SEQ ID NO:149; and a VL domain comprising a LCDR1, a LCDR2, and a LCDR3 of the amino acid sequence set forth in SEQ ID NO:150.

9. The binding agent of claim 8, wherein in the second binding region,
the VH domain of the second binding region comprises the HCDR1 comprising the amino acid sequence of SEQ ID NO:152, the HCDR2 comprising the amino acid sequence of SEQ ID NO: 153, and the HCDR3 comprising the amino acid sequence of SEQ ID NO:154; and the VL domain of the second binding region comprises the LCDR1 comprising the amino acid sequence of SEQ ID NO:155, the LCDR2 comprising the amino acid sequence of SEQ ID NO:156, and the LCDR3 comprising the amino acid sequence of SEQ ID NO: 157.

10. The binding agent of any of claims 1-9, wherein the first binding region comprises a VH domain comprising a HCDR1, a HCDR2, and a HCDR3 of the amino acid sequence set forth in SEQ ID NO:17, and a VL domain comprising a LCDR1, a LCDR2, and a LCDR3 of the amino acid sequence set forth in SEQ ID NO:18.

11. The binding agent of claim 10, wherein in the first binding region,
(a) the VH domain of the first binding region comprises the HCDR1 comprising the amino acid sequence of SEQ ID NO: 1, the HCDR2 comprising the amino acid sequence of SEQ ID NO:2, and the HCDR3 comprising the amino acid sequence of SEQ ID NO:3; and the VL domain of the first binding region comprises the LCDR1 comprising the amino acid sequence of SEQ ID NO:4, the LCDR2 comprising the amino acid sequence of SEQ ID NO:5, and the LCDR3 comprising the amino acid sequence of SEQ ID NO:6;
(b) the VH domain of the first binding region comprises the HCDR1 comprising the amino acid sequence of SEQ ID NO:7, the HCDR2 comprising the amino acid sequence of SEQ ID NO:8, and the HCDR3 comprising the amino acid sequence of SEQ ID NO:3; and the VL domain of the first binding region comprises the LCDR1 comprising the amino acid sequence of SEQ ID NO:4, the LCDR2 comprising the amino acid sequence of SEQ ID NO:5, and the LCDR3 comprising the amino acid sequence of SEQ ID NO:6;
(c) the VH domain of the first binding region comprises the HCDR1 comprising the amino acid sequence of SEQ ID NO:1, the HCDR2 comprising the amino acid sequence of SEQ ID NO:9, and the HCDR3 comprising the amino acid sequence of SEQ ID NO:3; and the VL domain of the first binding region comprises the LCDR1 comprising the amino acid sequence of SEQ ID NO:4, the LCDR2 comprising the amino acid sequence of SEQ ID NO:5, and the LCDR3 comprising the amino acid sequence of SEQ ID NO:6;
(d) the VH domain of the first binding region comprises the HCDR1 comprising the amino acid sequence of SEQ ID NO: 10, the HCDR2 comprising the amino acid sequence of SEQ ID NO: 2, and the HCDR3 comprising the amino acid sequence of SEQ ID NO:3; and the VL domain of the first binding region comprises the LCDR1 comprising the amino acid sequence of SEQ ID NO: 4, the LCDR2 comprising the amino acid sequence of SEQ ID NO:5, and the LCDR3 comprising the amino acid sequence of SEQ ID NO:6; or
(e) the VH domain of the first binding region comprises the HCDR1 comprising the amino acid sequence of SEQ ID NO:11, the HCDR2 comprising the amino acid sequence of SEQ ID NO: 12, and the HCDR3 comprising the amino acid sequence of SEQ ID NO: 13; and the VL domain of the first binding region comprises the LCDR1 comprising the amino acid sequence of SEQ ID NO:14, the LCDR2 comprising the amino acid sequence of SEQ ID NO: 15, and the LCDR3 comprising the amino acid sequence of SEQ ID NO: 16.

12. The binding agent of claim 10 or 11, wherein
(i) the first binding region comprises the VH domain having at least 90% sequence identity to the amino acid sequence of SEQ ID NO:17, and the VL domain having at least 90% sequence identity to the amino acid sequence of SEQ ID NO: 18; and the second binding region comprises the VH domain having at least 95% sequence identity to the amino acid sequence of SEQ ID NO: 149, and the VL domain having at least 90% sequence identity to the amino acid sequence of SEQ ID NO: 150; or
(ii) the first binding region comprises the VH domain comprising the amino acid sequence of SEQ ID NO: 17, and the VL domain comprising the amino acid sequence of SEQ ID NO: 18; and
the second binding region comprises the VH domain comprising the amino acid sequence of SEQ ID NO: 149, and the VL domain comprising the amino acid sequence of SEQ ID NO:150.

13. A binding agent comprising a first binding region that binds to human ILT3 and a second binding region that binds to human CD3, wherein the second binding region comprises a VH domain comprising a HCDR1, a HCDR2, and a HCDR3 of the amino acid sequence set forth in SEQ ID NO:149, and a VL domain comprising a LCDR1, a LCDR2, and a LCDR3 of the amino acid sequence set forth in SEQ ID NO:150.

14. The binding agent of claim 13, wherein the VH domain of the second binding region comprises the HCDR1 comprising the amino acid sequence of SEQ ID NO:152, the HCDR2 comprising the amino acid sequence of SEQ ID NO:153, and the HCDR3 comprising the amino acid sequence of SEQ ID NO:154; and the VL domain of the second binding region comprises the LCDR1 comprising the amino acid sequence of SEQ ID NO: 155; the LCDR2 comprising the amino acid sequence of SEQ ID NO: 156; and the LCDR3 comprising the amino acid sequence of SEQ ID NO: 157.

15. The binding agent of claim 13 or 14, wherein the first binding region comprises a VH domain comprising a HCDR1, a HCDR2, and a HCDR3 of the amino acid sequence set forth in SEQ ID NO:17, and a VL domain comprising a LCDR1, a LCDR2, and a LCDR3 of the amino acid sequence set forth in SEQ ID NO:18.

16. The binding agent of any one of claims 13-15, wherein in the first binding region,
(a) the VH domain of the first binding region comprises the HCDR1 comprising the amino acid sequence of SEQ ID NO: 1, the HCDR2 comprising the amino acid sequence of SEQ ID NO:2, and the HCDR3 comprising the amino acid sequence of SEQ ID NO:3; and the VL domain of the first binding region comprises the LCDR1 comprising the amino acid sequence of SEQ ID NO:4, the LCDR2 comprising the amino acid sequence of SEQ ID NO:5, and the LCDR3 comprising the amino acid sequence of SEQ ID NO:6;
(b) the VH domain of the first binding region comprises the HCDR1 comprising the amino acid sequence of SEQ ID NO:7, the HCDR2 comprising the amino acid sequence of SEQ ID NO:8, and the HCDR3 comprising the amino acid sequence of SEQ ID NO:3; and the VL domain of the first binding region comprises the LCDR1 comprising the amino acid sequence of SEQ ID NO:4, the LCDR2 comprising the amino acid sequence of SEQ ID NO:5, and the LCDR3 comprising the amino acid sequence of SEQ ID NO:6;
(c) the VH domain of the first binding region comprises the HCDR1 comprising the amino acid sequence of SEQ ID NO:1, the HCDR2 comprising the amino acid sequence of SEQ ID NO:9, and the HCDR3 comprising the amino acid sequence of SEQ ID NO:3; and the VL domain of the first binding region comprises the LCDR1 comprising the amino acid sequence of SEQ ID NO:4, the LCDR2 comprising the amino acid sequence of SEQ ID NO:5, and the LCDR3 comprising the amino acid sequence of SEQ ID NO:6;
(d) the VH domain of the first binding region comprises the HCDR1 comprising the amino acid sequence of SEQ ID NO: 10, the HCDR2 comprising the amino acid sequence of SEQ ID NO: 2, and the HCDR3 comprising the amino acid sequence of SEQ ID NO:3; and the VL domain of the first binding region comprises the LCDR1 comprising the amino acid sequence of SEQ ID NO: 4, the LCDR2 comprising the amino acid sequence of SEQ ID NO:5, and the LCDR3 comprising the amino acid sequence of SEQ ID NO:6; or
(e) the VH domain of the first binding region comprises the HCDR1 comprising the amino acid sequence of SEQ ID NO: 11, the HCDR2 comprising the amino acid sequence of SEQ ID NO: 12, and the HCDR3 comprising the amino acid sequence of SEQ ID NO: 13; and the VL domain of the first binding region comprises the LCDR1 comprising the amino acid sequence of SEQ ID NO:14, the LCDR2 comprising the amino acid sequence of SEQ ID NO: 15, and the LCDR3 comprising the amino acid sequence of SEQ ID NO: 16.

17. The binding agent of claim 15 or 16, wherein
(i) the first binding region comprises the VH domain having at least 90% sequence identity to the amino acid sequence of SEQ ID NO:17, and the VL domain having at least 90% sequence identity to the amino acid sequence of SEQ ID NO:18; and the second binding region comprises the VH domain having at least 95% sequence identity to the amino acid sequence of SEQ ID NO:149, and the VL domain having at least 90% sequence identity to the amino acid sequence of SEQ ID NO: 150; or
(ii) the first binding region comprises the VH domain comprising the amino acid sequence of SEQ ID NO: 17, and the VL domain comprising the amino acid sequence of SEQ ID NO: 18; and the second binding region comprises the VH domain comprising the amino acid sequence of SEQ ID NO: 149, and the VL domain comprising the amino acid sequence of SEQ ID NO:150.

18. The binding agent of any one of claims 13 to 17, wherein the first binding region comprises an anti-ILT3 Fab.

19. The binding agent of any one of claims 13 to 18, wherein the second binding region comprises an anti-CD3 scFv.

20. The binding agent of any one of claims 13 to 19, wherein the binding agent further comprises a Fc region.

21. The binding agent of claim 20, wherein the binding agent comprises:
(i) a first polypeptide comprising the anti-CD3 scFv, a first CH2 domain, and a first CH3 domain;
(ii) a second polypeptide comprising the VH domain of the first binding region, a CH1 domain, a second CH2 domain and a second CH3 domain; and
(iii) a third polypeptide comprising the VL domain of the first binding region and a CL domain,
wherein the VH domain of the first binding region, the CH1 domain, the VL domain of the first binding region, and the CL domain form the anti-ILT3 Fab, and the first CH2 domain, the second CH2 domain, the first CH3 domain, and the second CH3 domain form the Fc region.

22. The binding agent of claim 21, wherein the first polypeptide comprising one or more amino acid mutations that form an engineered cavity, and the second polypeptide comprising one or more amino acid mutations that form an engineered protuberance, and wherein the first polypeptide dimerizes with the second polypeptide via positioning of the protuberance into the cavity.

23. The binding agent of claim 21 or 22, wherein
(i) the first polypeptide comprises the amino acid sequence of SEQ ID NO:147, the second polypeptide comprises the amino acid sequence of SEQ ID NO:19, and the third polypeptide comprises the amino acid sequence of SEQ ID NO:20, or
(ii) the first polypeptide comprises an amino acid sequence having at least 90% sequence identity of the amino acid sequence of SEQ ID NO:147, the second polypeptide comprises an amino acid sequence having at least 90% sequence identity of the amino acid sequence of SEQ ID NO:19, and the third polypeptide comprises an amino acid sequence having at least 90% sequence identity of the amino acid sequence of SEQ ID NO:20.

24. The binding agent of claims 13 to 17, wherein the first binding region comprises two identical anti-ILT3 Fabs, and the second binding region comprises an anti-CD3 scFv.

25. The binding agent of claim 24, wherein the binding agent comprises:
(i) a first polypeptide comprising the ant-CD3 scFv, a first CH2 domain, and a first CH3 domain;
(ii) a second polypeptide comprising a first VH domain, a second VH domain, a first CH1 domain, a second CH1 domain, a second CH2 domain, and a second CH3 domain, wherein each of the first and second VH domains comprises the VH domain of the first binding region;
(iii) a third polypeptide comprising a first VL domain and a first CL domain, wherein the first VL domain comprises the VL domain of the first binding region; and
(iv) a fourth polypeptide comprising a second VL domain and a second CL domain, wherein the second VL domain comprises the VL domain of the first binding region,
wherein the first VH domain and the first CH1 domain of the second polypeptide and the first VL domain and the first CL domain of the third polypeptide form a first Fab region, the second VH domain and the second CH1 domain of the second polypeptide and the second VL domain and the second CL domain of the fourth polypeptide form a second Fab region, and the first CH2 domain, the second CH2 domain, the first CH3 domain, and the second CH3 domain form the Fc region.

26. The binding agent of claim 25, wherein the first polypeptide comprising one or more amino acid mutations that form an engineered cavity, and the second polypeptide comprising one or more amino acid mutations that form an engineered protuberance, and wherein the first polypeptide dimerizes with the second polypeptide via positioning of the protuberance into the cavity.

27. The binding agent of claim 25 or 26, wherein
(i) the first polypeptide comprises the amino acid sequence of SEQ ID NO:147, the second polypeptide comprises the amino acid sequence of SEQ ID NO:169, the third polypeptide comprises the amino acid sequence of SEQ ID NO:20, and the fourth polypeptide comprises the amino acid sequence of SEQ ID NO:20; or
(ii) the first polypeptide comprises an amino acid sequence having at least 90% sequence identity of the amino acid sequence of SEQ ID NO:147, the second polypeptide comprises an amino acid sequence having at least 90% sequence identity of the amino acid sequence of SEQ ID NO: 169, the third polypeptide comprises an amino acid sequence having at least 90% sequence identity of the amino acid sequence of SEQ ID NO:20, and the fourth polypeptide comprises an amino acid sequence having at least 90% sequence identity of the amino acid sequence of SEQ ID NO: 20.

28. The binding agent of any one of claims 1-12 and 19-27, wherein the anti-CD3 scFv comprises the amino acid sequence of SEQ ID NO:151.

29. The binding agent of any one of claims 1 to 28, wherein the binding agent is a humanized antibody.

30. A binding agent comprises:
(i) a first polypeptide comprising an scFv that binds to human CD3, a first CH2 domain, and a first CH3 domain;
(ii) a second polypeptide comprising a VH domain that binds to human ILT3, a CH1 domain, a second CH2 domain and a second CH3 domain; and
(iii) a third polypeptide comprising a VL domain that binds to human ILT3, and a CL domain,
wherein the scFv that binds to human CD3 comprises a VH domain comprising a HCDR1, a HCDR2, and a HCDR3 of the amino acid sequence set forth in SEQ ID NO:149, and a VL domain comprising a LCDR1, a LCDR2, and a LCDR3 of the amino acid sequence set forth in SEQ ID NO: 150; and
wherein the VH domain that binds to human ILT3 comprises a HCDR1, a HCDR2, and a HCDR3 of the amino acid sequence set forth in SEQ ID NO:17, and the VL domain that binds to human ILT3 comprises a LCDR1, a LCDR2, and a LCDR3 of the amino acid sequence set forth in SEQ ID NO:18.

31. The binding agent of claim 30, wherein:
(a) the HCDR1 of the scFv comprises the amino acid sequence of SEQ ID NO: 152, the HCDR2 of the scFv comprises the amino acid sequence of SEQ ID NO: 153, the HCDR3 of the scFv comprises the amino acid sequence of SEQ ID NO:154, the LCDR1 of the scFv comprises the amino acid sequence of SEQ ID NO:155, the LCDR2 of the scFv comprises the amino acid sequence of SEQ ID NO:156, and the LCDR3 of the scFv comprises the amino acid sequence of SEQ ID NO:157; and
(b) in the VH domain and VL domain that bind to human ILT3
(i) the HCDR1 comprises the amino acid sequence of SEQ ID NO:1; the HCDR2 comprises the amino acid sequence of SEQ ID NO:2; the HCDR3 comprises the amino acid sequence of SEQ ID NO:3; the LCDR1 comprises the amino acid sequence of SEQ ID NO:4; the LCDR2 comprises the amino acid sequence of SEQ ID NO:5; and the LCDR3 comprises the amino acid sequence of SEQ ID NO:6;
(ii) the HCDR1 comprises the amino acid sequence of SEQ ID NO:7; the HCDR2 comprises the amino acid sequence of SEQ ID NO:8; the HCDR3 comprises the amino acid sequence of SEQ ID NO:3; the LCDR1 comprises the amino acid sequence of SEQ ID NO:4; the LCDR2 comprises the amino acid sequence of SEQ ID NO:5; and the LCDR3 comprises the amino acid sequence of SEQ ID NO:6;
(iii) the HCDR1 comprises the amino acid sequence of SEQ ID NO:1; the HCDR2 comprises the amino acid sequence of SEQ ID NO:9; the HCDR3 comprises the amino acid sequence of SEQ ID NO:3; the LCDR1 comprises the amino acid sequence of SEQ ID NO:4; the LCDR2 comprises the amino acid sequence of SEQ ID NO:5; and the LCDR3 comprises the amino acid sequence of SEQ ID NO:6;
(iv) the HCDR1 comprises the amino acid sequence of SEQ ID NO: 10; the HCDR2 comprises the amino acid sequence of SEQ ID NO:2; the HCDR3 comprises the amino acid sequence of SEQ ID NO:3; the LCDR1 comprises the amino acid sequence of SEQ ID NO:4; the LCDR2 comprises the amino acid sequence of SEQ ID NO:5; and the LCDR3 comprises the amino acid sequence of SEQ ID NO:6; or
(v) the HCDR1 comprises the amino acid sequence of SEQ ID NO:11; the HCDR2 comprises the amino acid sequence of SEQ ID NO:12; the HCDR3 comprises the amino acid sequence of SEQ ID NO: 13; the LCDR1 comprises the amino acid sequence of SEQ ID NO: 14; the LCDR2 comprises the amino acid sequence of SEQ ID NO: 15; and the LCDR3 comprises the amino acid sequence of SEQ ID NO:16.

32. The binding agent of claim 30 or claim 31, wherein the VH domain of the scFv that binds to human CD3 comprises the amino acid sequence of SEQ ID NO: 149, and the VL domain of the scFv that binds to human CD3 comprises the amino acid sequence of SEQ ID NO:150; and the VH domain that binds to human ILT3 comprises the amino acid sequence of SEQ ID NO:17, and the VL domain that binds to human ILT3 comprises the amino acid sequence of SEQ ID NO:18.

33. The binding agent of any one of claims 30 to 32, wherein the scFv comprises the amino acid sequence of SEQ ID NO:151.

34. An isolated polynucleotide encoding the binding agent of any one of claims 1 to 33.

35. A vector comprising the polynucleotide of claim 34.

36. An isolated cell comprising the polynucleotide of claim 34 or the vector of claim 35.

37. An isolated cell producing the binding agent of any one of claims 1 to 33.

38. A pharmaceutical composition comprising the binding agent of any one of claims 1 to 33, the isolated polynucleotide of claim 34, the vector of claim 35, or the isolated cell of claim 36 or claim 37, and a pharmaceutically acceptable excipient.

39. A method of directing a T cell to a cancer or tumor cell expressing ILT3, comprising contacting the T cell with an effective amount of the binding agent of any one of claims 1 to 33 or the pharmaceutical composition of claim 38.

40. The method of claim 39, wherein the T cell induces the killing of the cancer or tumor cell expressing ILT3.

41. The method of claim 40, wherein the cancer or tumor cell is a hematological cancer or tumor cell.

42. The method of claim 41, wherein the hematological cancer or tumor cell is selected from the group consisting of an acute myeloid leukemia (AML) cell, a M4/M5 AML cell, a chronic myelomonocytic leukemia (CMML) cell, a B-cell acute lymphoblastic leukemia (B-ALL) cell, a chronic lymphocytic leukemia (CLL) cell, a diffuse large B-cell lymphoma (DLBCL) cell, a mantle cell lymphoma (MCL) cell, a multiple myeloma (MM) cell, a myelodysplastic syndrome (MDS) cell, a Hodgkin lymphoma cell, a lymphoplasmacytic lymphoma (LPL) cell, a follicular lymphoma cell, a Burkitt lymphoma cell, a blastic plasmacytoid dendritic cell neoplasm (BPDCN) cell, a marginal zone lymphoma cell, or a mucosa-associated lymphoid tissue (MALT) lymphoma cell.

43. The method of any one of claims 39 to 42, wherein the T cell fails to induce the killing of a normal hematopoietic stem cell (HSC).

44. A method of activating a T cell, comprising contacting the T cell with an effective amount of the binding agent of any one of claims 1 to 33 or the pharmaceutical composition of claim 38, wherein the second binding region binds to the T cell.

45. The method of claim 44, wherein the T cell is a naïve T cell.

46. The method of claim 44 or claim 45, wherein the T cell is polyclonally expanded from a population of PBMCs.

47. A method of killing or inhibiting the proliferation of a cancer or tumor cell expressing ILT3, comprising contacting the cancer or tumor cell with the binding agent of any one of claims 1 to 33 or the pharmaceutical composition of claim 38.

48. The method of claim 47, wherein the binding agent activates a T cell.

49. The method of claim 48, wherein the activated T cell induces the killing of the cancer or tumor cell.

50. The method of any one of claims 47 to 49, wherein the cancer or tumor cell comprises a hematological cancer or tumor cell.

51. The method of claim 50, wherein the hematological cancer or tumor cell is selected from the group consisting of an acute myeloid leukemia (AML) cell, a M4/M5 AML cell, a chronic myelomonocytic leukemia (CMML) cell, a B-cell acute lymphoblastic leukemia (B-ALL) cell, a chronic lymphocytic leukemia (CLL) cell, a diffuse large B-cell lymphoma (DLBCL) cell, a mantle cell lymphoma (MCL) cell, a multiple myeloma (MM) cell, a myelodysplastic syndrome (MDS) cell, a Hodgkin lymphoma cell, a lymphoplasmacytic lymphoma (LPL) cell, a follicular lymphoma cell, a Burkitt lymphoma cell, a blastic plasmacytoid dendritic cell neoplasm (BPDCN) cell, a marginal zone lymphoma cell, or a mucosa-associated lymphoid tissue (MALT) lymphoma cell.

52. A method of treating a cancer or a tumor expressing ILT3 in a subject, comprising administering an effective amount of the binding agent of any one of claims 1 to 33 or the pharmaceutical composition of claim 38 to the subject.

53. The method of claim 52, wherein the cancer or tumor comprises a hematological cancer or tumor.

54. The method of claim 53, wherein the hematological cancer or tumor is selected from the group consisting of acute myeloid leukemia (AML), a M4/M5 AML chronic myelomonocytic leukemia (CMML), B-cell acute lymphoblastic leukemia (B-ALL), chronic lymphocytic leukemia (CLL), diffuse large B-cell lymphoma (DLBCL), mantle cell lymphoma (MCL), multiple myeloma (MM), myelodysplastic syndrome (MDS), Hodgkin lymphoma, lymphoplasmacytic lymphoma (LPL), follicular lymphoma, Burkitt lymphoma, blastic plasmacytoid dendritic cell neoplasm (BPDCN), marginal zone lymphoma, or mucosa-associated lymphoid tissue (MALT) lymphoma.


SEQUENCE LISTING (169 sequences)

SEQ ID NO: 1 - protein, 10 aa, synthetic construct
  source 1..10: note = Hz45G10 Heavy Chain variable region CDR1
GFTFSDYGMH

SEQ ID NO: 2 - protein, 17 aa, synthetic construct
  source 1..17: note = Hz45G10 Heavy Chain variable region CDR2
YIFSGSSTIYYADTVKG

SEQ ID NO: 3 - protein, 9 aa, synthetic construct
  source 1..9: note = Hz45G10 Heavy Chain variable region CDR3
ADGRGAMDY

SEQ ID NO: 4 - protein, 11 aa, synthetic construct
  source 1..11: note = Hz45G10 Light Chain variable region CDR1
RASQDISKFLN

SEQ ID NO: 5 - protein, 7 aa, synthetic construct
  source 1..7: note = Hz45G10 Light Chain variable region CDR2
YTSRLHS

SEQ ID NO: 6 - protein, 9 aa, synthetic construct
  source 1..9: note = Hz45G10 Light Chain variable region CDR3
QQGNTLPWT

SEQ ID NO: 7 - protein, 7 aa, synthetic construct
  source 1..7: note = Hz45G10 Heavy Chain variable region CDR1
GFTFSDY

SEQ ID NO: 8 - protein, 6 aa, synthetic construct
  source 1..6: note = Hz45G10 Heavy Chain variable region CDR2
FSGSST

SEQ ID NO: 9 - protein, 10 aa, synthetic construct
  source 1..10: note = Hz45G10 Heavy Chain variable region CDR2
YIFSGSSTIY

SEQ ID NO: 10 - protein, 5 aa, synthetic construct
  source 1..5: note = Hz45G10 Heavy Chain variable region CDR1
DYGMH

SEQ ID NO: 11 - protein, 6 aa, synthetic construct
  source 1..6: note = Hz45G10 Heavy Chain variable region CDR1
SDYGMH

SEQ ID NO: 12 - protein, 13 aa, synthetic construct
  source 1..13: note = Hz45G10 Heavy Chain variable region CDR2
WVAYIFSGSSTIY

SEQ ID NO: 13 - protein, 10 aa, synthetic construct
  source 1..10: note = Hz45G10 Heavy Chain variable region CDR3
ARADGRGAMD

SEQ ID NO: 14 - protein, 7 aa, synthetic construct
  source 1..7: note = Hz45G10 Light Chain variable region CDR1
SKFLNWY

SEQ ID NO: 15 - protein, 10 aa, synthetic construct
  source 1..10: note = Hz45G10 Light Chain variable region CDR2
LLIYYTSRLH

SEQ ID NO: 16 - protein, 8 aa, synthetic construct
  source 1..8: note = Hz45G10 Light Chain variable region CDR3
QQGNTLPW

SEQ ID NO: 17 - protein, 118 aa, synthetic construct
  source 1..118: note = Hz45G10 Heavy chain variable region amino acid sequence (VH)
EVQLVESGGGLVQPGGSLRLSCAASGFTFSDYGMHWVRQAPGKGLEWVAYIFSGSSTIYYADTVKGRFTISRDNAKNSLYLQMNSLRAEDTAVYYCARADGRGAMDYWGQGTLVTVSS

SEQ ID NO: 18 - protein, 107 aa, synthetic construct
  source 1..107: note = Hz45G10 Light chain variable region amino acid sequence (VL)
DIQMTQSPSSLSASVGDRVTITCRASQDISKFLNWYQQKPGKAPKLLIYYTSRLHSGVPSRFSGSGSGTDFTFTISSLQPEDIATYFCQQGNTLPWTFGGGTKLEIK

SEQ ID NO: 19 - protein, 448 aa, synthetic construct
  source 1..448: note = Hz45G10 Heavy chain amino acid sequence
EVQLVESGGGLVQPGGSLRLSCAASGFTFSDYGMHWVRQAPGKGLEWVAYIFSGSSTIYYADTVKGRFTISRDNAKNSLYLQMNSLRAEDTAVYYCARADGRGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, synthetic construct
  source 1..214: note = Hz45G10 Light chain amino acid sequence
DIQMTQSPSSLSASVGDRVTITCRASQDISKFLNWYQQKPGKAPKLLIYYTSRLHSGVPSRFSGSGSGTDFTFTISSLQPEDIATYFCQQGNTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 10 aa, synthetic construct
  source 1..10: note = 3A3 Heavy chain variable region CDR1
GFSLTSYGVH

SEQ ID NO: 22 - protein, 16 aa, synthetic construct
  source 1..16: note = 3A3 Heavy chain variable region CDR2
VIWPGGTINYNSALMS

SEQ ID NO: 23 - protein, 10 aa, synthetic construct
  source 1..10: note = 3A3 Heavy chain variable region CDR3
DKYDGGWFAY

SEQ ID NO: 24 - protein, 11 aa, synthetic construct
  source 1..11: note = 3A3 Light chain variable region CDR1
KASQNVRTAVA

SEQ ID NO: 25 - protein, 7 aa, synthetic construct
  source 1..7: note = 3A3 Light chain variable region CDR2
LASNRHT

SEQ ID NO: 26 - protein, 9 aa, synthetic construct
  source 1..9: note = 3A3 Light chain variable region CDR3
LQHLNYPLT

SEQ ID NO: 27 - protein, 7 aa, synthetic construct
  source 1..7: note = 3A3 Heavy chain variable region CDR1
GFSLTSY

SEQ ID NO: 28 - protein, 5 aa, synthetic construct
  source 1..5: note = 3A3 Heavy chain variable region CDR2
WPGGT

SEQ ID NO: 29 - protein, 9 aa, synthetic construct
  source 1..9: note = 3A3 Heavy chain variable region CDR2
VIWPGGTIN

SEQ ID NO: 30 - protein, 5 aa, synthetic construct
  source 1..5: note = 3A3 Heavy chain variable region CDR1
SYGVH

SEQ ID NO: 31 - protein, 6 aa, synthetic construct
  source 1..6: note = 3A3 Heavy chain variable region CDR1
TSYGVH

SEQ ID NO: 32 - protein, 12 aa, synthetic construct
  source 1..12: note = 3A3 Heavy chain variable region CDR2
WLGVIWPGGTIN

SEQ ID NO: 33 - protein, 11 aa, synthetic construct
  source 1..11: note = 3A3 Heavy chain variable region CDR3
ASDKYDGGWFA

SEQ ID NO: 34 - protein, 7 aa, synthetic construct
  source 1..7: note = 3A3 Light chain variable region CDR1
RTAVAWY

SEQ ID NO: 35 - protein, 10 aa, synthetic construct
  source 1..10: note = 3A3 Light chain variable region CDR2
ALIYLASNRH

SEQ ID NO: 36 - protein, 8 aa, synthetic construct
  source 1..8: note = 3A3 Light chain variable region CDR3
LQHLNYPL

SEQ ID NO: 37 - protein, 118 aa, synthetic construct
  source 1..118: note = 3A3 Heavy chain variable region amino acid sequence
QVQLKESGPGLVAPSQSLSITCTVSGFSLTSYGVHWVRQPPGKGLEWLGVIWPGGTINYNSALMSRLSISKDNSKSQVFLKLNSLQTDDTAMYYCASDKYDGGWFAYWGQGTLVTVSA

SEQ ID NO: 38 - protein, 107 aa, synthetic construct
  source 1..107: note = 3A3 Light chain variable region amino acid sequence
DIVMTQSQKFMSTSVGDRVSITCKASQNVRTAVAWYQQKPGQSPEALIYLASNRHTGVPDRFTGSGSGTDFSLSISNVQSEDLADYFCLQHLNYPLTFGSGTKLEIK

SEQ ID NO: 39 - protein, 10 aa, synthetic construct
  source 1..10: note = 5A7 Heavy chain variable region CDR1
GFTFSSYGMS

SEQ ID NO: 40 - protein, 17 aa, synthetic construct
  source 1..17: note = 5A7 Heavy chain variable region CDR2
TISGGGSYTNYPDSVKG

SEQ ID NO: 41 - protein, 12 aa, synthetic construct
  source 1..12: note = 5A7 Heavy chain variable region CDR3
REWRMTLYAMDY

SEQ ID NO: 42 - protein, 15 aa, synthetic construct
  source 1..15: note = 5A7 Light chain variable region CDR1
RASESVDSYGNSFMH

SEQ ID NO: 43 - protein, 7 aa, synthetic construct
  source 1..7: note = 5A7 Light chain variable region CDR2
LTSNLES

SEQ ID NO: 44 - protein, 9 aa, synthetic construct
  source 1..9: note = 5A7 Light chain variable region CDR3
QQNNEDPFT

SEQ ID NO: 45 - protein, 7 aa, synthetic construct
  source 1..7: note = 5A7 Heavy chain variable region CDR1
GFTFSSY

SEQ ID NO: 46 - protein, 6 aa, synthetic construct
  source 1..6: note = 5A7 Heavy chain variable region CDR2
SGGGSY

SEQ ID NO: 47 - protein, 10 aa, synthetic construct
  source 1..10: note = 5A7 Heavy chain variable region CDR2
TISGGGSYTN

SEQ ID NO: 48 - protein, 5 aa, synthetic construct
  source 1..5: note = 5A7 Heavy chain variable region CDR1
SYGMS

SEQ ID NO: 49 - protein, 6 aa, synthetic construct
  source 1..6: note = 5A7 Heavy chain variable region CDR1
SSYGMS

SEQ ID NO: 50 - protein, 13 aa, synthetic construct
  source 1..13: note = 5A7 Heavy chain variable region CDR2
WVATISGGGSYTN

SEQ ID NO: 51 - protein, 13 aa, synthetic construct
  source 1..13: note = 5A7 Heavy chain variable region CDR3
ARREWRMTLYAMD

SEQ ID NO: 52 - protein, 11 aa, synthetic construct
  source 1..11: note = 5A7 Light chain variable region CDR1
DSYGNSFMHWY

SEQ ID NO: 53 - protein, 10 aa, synthetic construct
  source 1..10: note = 5A7 Light chain variable region CDR2
LLIYLTSNLE

SEQ ID NO: 54 - protein, 8 aa, synthetic construct
  source 1..8: note = 5A7 Light chain variable region CDR3
QQNNEDPF

SEQ ID NO: 55 - protein, 121 aa, synthetic construct
  source 1..121: note = 5A7 Heavy chain variable region amino acid sequence
EVKLVESGGGLVKPGGSLKLSCAASGFTFSSYGMSWVRQTPEKRLEWVATISGGGSYTNYPDSVKGRLTISRDNAKKNLYLEMSSLRSEDTALYYCARREWRMTLYAMDYWGQGTSVTVSS

SEQ ID NO: 56 - protein, 111 aa, synthetic construct
  source 1..111: note = 5A7 Light chain variable region amino acid sequence
NIVLTQSPASLAVSLGQRATISCRASESVDSYGNSFMHWYQQKPGQAPKLLIYLTSNLESGVPARFSGSGSRTDFTLTIDPVEADDAATYYCQQNNEDPFTFGSGTKLEIK

SEQ ID NO: 57 - protein, 10 aa, synthetic construct
  source 1..10: note = 12A12 Heavy chain variable region CDR1
GYTFTDYNMD

SEQ ID NO: 58 - protein, 17 aa, synthetic construct
  source 1..17: note = 12A12 Heavy chain variable region CDR2
YIYPNNGGTGYNQKFNS

SEQ ID NO: 59 - protein, 14 aa, synthetic construct
  source 1..14: note = 12A12 Heavy chain variable region CDR3
SPYYDYVGSYAMDY

SEQ ID NO: 60 - protein, 12 aa, synthetic construct
  source 1..12: note = 12A12 Light chain variable region CDR1
TASSSVSSSYLH

SEQ ID NO: 61 - protein, 7 aa, synthetic construct
  source 1..7: note = 12A12 Light chain variable region CDR2
STSNLAS

SEQ ID NO: 62 - protein, 9 aa, synthetic construct
  source 1..9: note = 12A12 Light chain variable region CDR3
HQYHRSPRT

SEQ ID NO: 63 - protein, 7 aa, synthetic construct
  source 1..7: note = 12A12 Heavy chain variable region CDR1
GYTFTDY

SEQ ID NO: 64 - protein, 6 aa, synthetic construct
  source 1..6: note = 12A12 Heavy chain variable region CDR2
YPNNGG

SEQ ID NO: 65 - protein, 10 aa, synthetic construct
  source 1..10: note = 12A12 Heavy chain variable region CDR2
YIYPNNGGTG

SEQ ID NO: 66 - protein, 5 aa, synthetic construct
  source 1..5: note = 12A12 Heavy chain variable region CDR1
DYNMD

SEQ ID NO: 67 - protein, 6 aa, synthetic construct
  source 1..6: note = 12A12 Heavy chain variable region CDR1
TDYNMD

SEQ ID NO: 68 - protein, 13 aa, synthetic construct
  source 1..13: note = 12A12 Heavy chain variable region CDR2
WIGYIYPNNGGTG

SEQ ID NO: 69 - protein, 15 aa, synthetic construct
  source 1..15: note = 12A12 Heavy chain variable region CDR3
ASSPYYDYVGSYAMD

SEQ ID NO: 70 - protein, 8 aa, synthetic construct
  source 1..8: note = 12A12 Light chain variable region CDR1
SSSYLHWY

SEQ ID NO: 71 - protein, 10 aa, synthetic construct
  source 1..10: note = 12A12 Light chain variable region CDR2
LWIYSTSNLA

SEQ ID NO: 72 - protein, 8 aa, synthetic construct
  source 1..8: note = 12A12 Light chain variable region CDR3
HQYHRSPR

SEQ ID NO: 73 - protein, 123 aa, synthetic construct
  source 1..123: note = 12A12 Heavy chain variable region amino acid sequence
EVQLQQSGPELVKPGASVKISCKASGYTFTDYNMDWVKQSHGKSLEWIGYIYPNNGGTGYNQKFNSKATLTVDKSSSTAYMELHSLTSEDSAVYYCASSPYYDYVGSYAMDYWGQGTSVTVSS

SEQ ID NO: 74 - protein, 108 aa, synthetic construct
  source 1..108: note = 12A12 Light chain variable region amino acid sequence
QIVLTQSPAIMSASLGERVTMTCTASSSVSSSYLHWYQQKPGSSPKLWIYSTSNLASGVPARFSGSGSGTSYSLTISSMEAEDAATYYCHQYHRSPRTFGGGTKLEIK

SEQ ID NO: 75 - protein, 10 aa, synthetic construct
  source 1..10: note = 16C5 Heavy chain variable region CDR1
GYTFTDYNMD

SEQ ID NO: 76 - protein, 17 aa, synthetic construct
  source 1..17: note = 16C5 Heavy chain variable region CDR2
YIYPSNGGTGYNQKFKS

SEQ ID NO: 77 - protein, 14 aa, synthetic construct
  source 1..14: note = 16C5 Heavy chain variable region CDR3
VPYYDYLYYYAMDY

SEQ ID NO: 78 - protein, 10 aa, synthetic construct
  source 1..10: note = 16C5 Light chain variable region CDR1
RASSSVSFMH

SEQ ID NO: 79 - protein, 7 aa, synthetic construct
  source 1..7: note = 16C5 Light chain variable region CDR2
ATSNLAS

SEQ ID NO: 80 - protein, 11 aa, synthetic construct
  source 1..11: note = 16C5 Light chain variable region CDR3
QQWSTNPYMYT

SEQ ID NO: 81 - protein, 7 aa, synthetic construct
  source 1..7: note = 16C5 Heavy chain variable region CDR1
GYTFTDY

SEQ ID NO: 82 - protein, 6 aa, synthetic construct
  source 1..6: note = 16C5 Heavy chain variable region CDR2
YPSNGG

SEQ ID NO: 83 - protein, 10 aa, synthetic construct
  source 1..10: note = 16C5 Heavy chain variable region CDR2
YIYPSNGGTG

SEQ ID NO: 84 - protein, 5 aa, synthetic construct
  source 1..5: note = 16C5 Heavy chain variable region CDR1
DYNMD

SEQ ID NO: 85 - protein, 6 aa, synthetic construct
  source 1..6: note = 16C5 Heavy chain variable region CDR1
TDYNMD

SEQ ID NO: 86 - protein, 13 aa, synthetic construct
  source 1..13: note = 16C5 Heavy chain variable region CDR2
WIGYIYPSNGGTG

SEQ ID NO: 87 - protein, 15 aa, synthetic construct
  source 1..15: note = 16C5 Heavy chain variable region CDR3
ARVPYYDYLYYYAMD

SEQ ID NO: 88 - protein, 6 aa, synthetic construct
  source 1..6: note = 16C5 Light chain variable region CDR1
SFMHWY

SEQ ID NO: 89 - protein, 10 aa, synthetic construct
  source 1..10: note = 16C5 Light chain variable region CDR2
PWIYATSNLA

SEQ ID NO: 90 - protein, 10 aa, synthetic construct
  source 1..10: note = 16C5 Light chain variable region CDR3
QQWSTNPYMY

SEQ ID NO: 91 - protein, 123 aa, synthetic construct
  source 1..123: note = 16C5 Heavy chain variable region amino acid sequence
EVQLQQSGPELVKPGASVKISCKASGYTFTDYNMDWVKQSHGKSLEWIGYIYPSNGGTGYNQKFKSKATLTVDKSSNTAYMELHSLTSEDSAVYYCARVPYYDYLYYYAMDYWGQGTSVTVSS

SEQ ID NO: 92 - protein, 108 aa, synthetic construct
  source 1..108: note = 16C5 Light chain variable region amino acid sequence
QIVLSQSPAILSASPGEKVTMACRASSSVSFMHWYQQKPGSSPQPWIYATSNLASGVPARFSGSGSGTSYSLTISRVEAEDAATYYCQQWSTNPYMYTFGGGTKLEIK

SEQ ID NO: 93 - protein, 10 aa, synthetic construct
  source 1..10: note = 48A6 Heavy chain variable region CDR1
GFTFSSYGMS

SEQ ID NO: 94 - protein, 17 aa, synthetic construct
  source 1..17: note = 48A6 Heavy chain variable region CDR2
TISSGGTYTFYPDSVKG

SEQ ID NO: 95 - protein, 12 aa, synthetic construct
  source 1..12: note = 48A6 Heavy chain variable region CDR3
RGWLLHYYAMDY

SEQ ID NO: 96 - protein, 15 aa, synthetic construct
  source 1..15: note = 48A6 Light chain variable region CDR1
RPSESVDSFGNSFMH

SEQ ID NO: 97 - protein, 7 aa, synthetic construct
  source 1..7: note = 48A6 Light chain variable region CDR2
LSSKLES

SEQ ID NO: 98 - protein, 9 aa, synthetic construct
  source 1..9: note = 48A6 Light chain variable region CDR3
QQHNEDPFT

SEQ ID NO: 99 - protein, 7 aa, synthetic construct
  source 1..7: note = 48A6 Heavy chain variable region CDR1
GFTFSSY

SEQ ID NO: 100 - protein, 6 aa, synthetic construct
  source 1..6: note = 48A6 Heavy chain variable region CDR2
SSGGTY

SEQ ID NO: 101 - protein, 10 aa, synthetic construct
  source 1..10: note = 48A6 Heavy chain variable region CDR2
TISSGGTYTF

SEQ ID NO: 102 - protein, 5 aa, synthetic construct
  source 1..5: note = 48A6 Heavy chain variable region CDR1
SYGMS

SEQ ID NO: 103 - protein, 6 aa, synthetic construct
  source 1..6: note = 48A6 Heavy chain variable region CDR1
SSYGMS

SEQ ID NO: 104 - protein, 13 aa, synthetic construct
  source 1..13: note = 48A6 Heavy chain variable region CDR2
WVATISSGGTYTF

SEQ ID NO: 105 - protein, 13 aa, synthetic construct
  source 1..13: note = 48A6 Heavy chain variable region CDR3
ARRGWLLHYYAMD

SEQ ID NO: 106 - protein, 11 aa, synthetic construct
  source 1..11: note = 48A6 Light chain variable region CDR1
DSFGNSFMHWF

SEQ ID NO: 107 - protein, 10 aa, synthetic construct
  source 1..10: note = 48A6 Light chain variable region CDR2
LLIYLSSKLE

SEQ ID NO: 108 - protein, 8 aa, synthetic construct
  source 1..8: note = 48A6 Light chain variable region CDR3
QQHNEDPF

SEQ ID NO: 109 - protein, 121 aa, synthetic construct
  source 1..121: note = 48A6 Heavy chain variable region amino acid sequence
EVQLVESGGDLMKPGGSLKLSCAASGFTFSSYGMSWVRQTPDKRLEWVATISSGGTYTFYPDSVKGRFTISRDNAKNTLYLQMSSLKSEDTAMYYCARRGWLLHYYAMDYWGQGTSVTVSS

SEQ ID NO: 110 - protein, 111 aa, synthetic construct
  source 1..111: note = 48A6 Light chain variable region amino acid sequence
NIVLTQSPASLAVSLGQRATISCRPSESVDSFGNSFMHWFQQKPGQPPKLLIYLSSKLESGVPARFSGSGSRTDFTLTIDPVEADDAATYYCQQHNEDPFTFGSGTKLEIK

SEQ ID NO: 111 - protein, 10 aa, synthetic construct
  source 1..10: note = 53F10 Heavy chain variable region CDR1
GFTFSDYGMH

SEQ ID NO: 112 - protein, 17 aa, synthetic construct
  source 1..17: note = 53F10 Heavy chain variable region CDR2
YISTGIITVYYADTVKG

SEQ ID NO: 113 - protein, 9 aa, synthetic construct
  source 1..9: note = 53F10 Heavy chain variable region CDR3
ADGRGAMDY

SEQ ID NO: 114 - protein, 11 aa, synthetic construct
  source 1..11: note = 53F10 Light chain variable region CDR1
RASQDISNFLN

SEQ ID NO: 115 - protein, 7 aa, synthetic construct
  source 1..7: note = 53F10 Light chain variable region CDR2
YTSRLHS

SEQ ID NO: 116 - protein, 9 aa, synthetic construct
  source 1..9: note = 53F10 Light chain variable region CDR3
QQGNTLPWT

SEQ ID NO: 117 - protein, 7 aa, synthetic construct
  source 1..7: note = 53F10 Heavy chain variable region CDR1
GFTFSDY

SEQ ID NO: 118 - protein, 6 aa, synthetic construct
  source 1..6: note = 53F10 Heavy chain variable region CDR2
STGIIT

SEQ ID NO: 119 - protein, 10 aa, synthetic construct
  source 1..10: note = 53F10 Heavy chain variable region CDR2
YISTGIITVY

SEQ ID NO: 120 - protein, 5 aa, synthetic construct
  source 1..5: note = 53F10 Heavy chain variable region CDR1
DYGMH

SEQ ID NO: 121 - protein, 6 aa, synthetic construct
  source 1..6: note = 53F10 Heavy chain variable region CDR1
SDYGMH

SEQ ID NO: 122 - protein, 13 aa, synthetic construct
  source 1..13: note = 53F10 Heavy chain variable region CDR2
WVAYISTGIITVY

SEQ ID NO: 123 - protein, 10 aa, synthetic construct
  source 1..10: note = 53F10 Heavy chain variable region CDR3
ARADGRGAMD

SEQ ID NO: 124 - protein, 7 aa, synthetic construct
  source 1..7: note = 53F10 Light chain variable region CDR1
SNFLNWY

SEQ ID NO: 125 - protein, 10 aa, synthetic construct
  source 1..10: note = 53F10 Light chain variable region CDR2
LLIYYTSRLH

SEQ ID NO: 126 - protein, 8 aa, synthetic construct
  source 1..8: note = 53F10 Light chain variable region CDR3
QQGNTLPW

SEQ ID NO: 127 - protein, 118 aa, synthetic construct
  source 1..118: note = 53F10 Heavy chain variable region amino acid sequence
EVQVVESGGGLVKPGGSLKLSCAASGFTFSDYGMHWVRQAPEKGLEWVAYISTGIITVYYADTVKGRFTMSRDNAKNTLFLQMTSLRSEDTAIYYCARADGRGAMDYWGQGTSVIVSS

SEQ ID NO: 128 - protein, 107 aa, synthetic construct
  source 1..107: note = 53F10 Light chain variable region amino acid sequence
DIQMTQTTSSLSASLGDRVTISCRASQDISNFLNWYQQKPDGTVTLLIYYTSRLHSGVPSRFSGSGSGTDYSLTISNLEQEDFATYFCQQGNTLPWTFGGGTKLEIK

SEQ ID NO: 129 - protein, 10 aa, synthetic construct
  source 1..10: note = Hz5A7.v5 Heavy chain variable region CDR1
GFTFSSYGMS

SEQ ID NO: 130 - protein, 17 aa, synthetic construct
  source 1..17: note = Hz5A7.v5 Heavy chain variable region CDR2
TISGGGSYTNYPDSVKG

SEQ ID NO: 131 - protein, 12 aa, synthetic construct
  source 1..12: note = Hz5A7.v5 Heavy chain variable region CDR3
REWRYTLYAMDY

SEQ ID NO: 132 - protein, 15 aa, synthetic construct
  source 1..15: note = Hz5A7.v5 Light chain variable region CDR1
RASESVESYGSSFMH

SEQ ID NO: 133 - protein, 7 aa, synthetic construct
  source 1..7: note = Hz5A7.v5 Light chain variable region CDR2
LTSNLES

SEQ ID NO: 134 - protein, 9 aa, synthetic construct
  source 1..9: note = Hz5A7.v5 Light chain variable region CDR3
QQNNEDPFT

SEQ ID NO: 135 - protein, 7 aa, synthetic construct
  source 1..7: note = Hz5A7.v5 Heavy chain variable region CDR1
GFTFSSY

SEQ ID NO: 136 - protein, 6 aa, synthetic construct
  source 1..6: note = Hz5A7.v5 Heavy chain variable region CDR2
SGGGSY

SEQ ID NO: 137 - protein, 10 aa, synthetic construct
  source 1..10: note = Hz5A7.v5 Heavy chain variable region CDR2
TISGGGSYTN

SEQ ID NO: 138 - protein, 5 aa, synthetic construct
  source 1..5: note = Hz5A7.v5 Heavy chain variable region CDR1
SYGMS

SEQ ID NO: 139 - protein, 6 aa, synthetic construct
  source 1..6: note = Hz5A7.v5 Heavy chain variable region CDR1
SSYGMS

SEQ ID NO: 140 - protein, 13 aa, synthetic construct
  source 1..13: note = Hz5A7.v5 Heavy chain variable region CDR2
WVATISGGGSYTN

SEQ ID NO: 141 - protein, 13 aa, synthetic construct
  source 1..13: note = Hz5A7.v5 Heavy chain variable region CDR3
ARREWRYTLYAMD

SEQ ID NO: 142 - protein, 11 aa, synthetic construct
  source 1..11: note = Hz5A7.v5 Light chain variable region CDR1
ESYGSSFMHWY

SEQ ID NO: 143 - protein, 10 aa, synthetic construct
  source 1..10: note = Hz5A7.v5 Light chain variable region CDR2
LLIYLTSNLE

SEQ ID NO: 144 - protein, 8 aa, synthetic construct
  source 1..8: note = Hz5A7.v5 Light chain variable region CDR3
QQNNEDPF

SEQ ID NO: 145 - protein, 121 aa, synthetic construct
  source 1..121: note = Hz5A7.v5 Heavy chain variable region amino acid sequence
EVQLVESGGGLVQPGGSLRLSCAASGFTFSSYGMSWVRQAPGKGLEWVATISGGGSYTNYPDSVKGRFTISRDNAKNSLYLQMNSLRAEDTAVYYCARREWRYTLYAMDYWGQGTTVTVSS

SEQ ID NO: 146 - protein, 111 aa, synthetic construct
  source 1..111: note = Hz5A7.v5 Light chain variable region amino acid sequence
DIQLTQSPSFLSASVGDRVTITCRASESVESYGSSFMHWYQQKPGKAPKLLIYLTSNLESGVPSRFSGSGSGTEFTLTISSLQPEDFATYYCQQNNEDPFTFGQGTKLEIK

SEQ ID NO: 147 - protein, 481 aa, synthetic construct
  source 1..481: note = CD3 scFv-Fc amino acid sequence
EVQLLESGGGLVQPGGSLKLSCAASGFTFNTYAMNWVRQAPGKCLEWVARIRSKYNNYATYYADSVKDRFTISRDDSKNTAYLQMNNLKTEDTAVYYCVRHGNFGNSYVSWFAYWGQGTLVTVSSGGGGSGGGGSGGGGSELVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGCGTKLTVLGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 148 - DNA, 1443 bp, synthetic construct
  source 1..1443: mol_type = other DNA; note = CD3 scFv-Fc nucleic acid sequence
gaggtgcagctgttggaatctggcggaggattggttcagcctggcggctctctgaagctgtcttgtgccgcttctggcttcaccttcaacacctacgccatgaactgggtccgacaggcccctggcaaatgcctggaatgggtcgcccggatcagatccaagtacaacaattacgccacctactacgccgactccgtgaaggaccggttcaccatctctcgggacgactccaagaacaccgcctacctgcagatgaacaacctcaagaccgaggataccgccgtgtactactgtgtgcggcacggcaacttcggcaactcctatgtgtcttggtttgcctactggggccagggcacactggtcacagtttctagcggcggaggtggaagcggaggcggaggtagtggtggtggcggatctgaactggtggtcacccaagagcctagcctgacagtttctcctggcggcaccgtgacactgacctgtagatcttctaccggcgctgtgaccacctccaactacgccaattgggtgcagcagaagccaggccaggctcctagaggactgatcggcggcacaaacaagagagcccctggaactcctgccaggttctctggatctctgctcggcggaaaggctgctctgacactgtctggtgtccagcctgaggacgaggccgagtattactgtgccctgtggtactccaacctgtgggtgttcggctgtggcaccaagctgacagttctcggaggcggcggatccgacaagacccatacttgtcctccatgtcctgctccagaggctgctggtggcccttccgtgtttctgttccctccaaagcctaaggacaccctgatgatctctcggacccctgaagtgacctgcgtggtggtcgatgtgtctcacgaggacccagaagtgaagttcaattggtacgtggacggcgtggaagtgcataacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccaagccgggaagagatgaccaagaaccaggtgtccctgtcctgtgccgtgaagggcttctacccttccgatatcgccgtggaatgggagagcaatggccagccagagaacaactacaagacaacccctcctgtgctggactccgacggctcattcttcctggtgtctaagctgactgtggacaagtccagatggcagcagggcaacgtgttctcctgctccgtgatgcacgaggccctgcacaatcactacacacagaagtccctgagcctgtctcctggcaag

SEQ ID NO: 149 - protein, 125 aa, synthetic construct
  source 1..125: note = CD3 VH amino acid sequence
EVQLLESGGGLVQPGGSLKLSCAASGFTFNTYAMNWVRQAPGKCLEWVARIRSKYNNYATYYADSVKDRFTISRDDSKNTAYLQMNNLKTEDTAVYYCVRHGNFGNSYVSWFAYWGQGTLVTVSS

SEQ ID NO: 150 - protein, 109 aa, synthetic construct
  source 1..109: note = CD3 VL amino acid sequence
ELVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGCGTKLTVL

SEQ ID NO: 151 - protein, 249 aa, synthetic construct
  source 1..249: note = CD3 scFv amino acid sequence
EVQLLESGGGLVQPGGSLKLSCAASGFTFNTYAMNWVRQAPGKCLEWVARIRSKYNNYATYYADSVKDRFTISRDDSKNTAYLQMNNLKTEDTAVYYCVRHGNFGNSYVSWFAYWGQGTLVTVSSGGGGSGGGGSGGGGSELVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGCGTKLTVL

SEQ ID NO: 152 - protein, 10 aa, synthetic construct
  source 1..10: note = CD3 Heavy Chain variable region CDR1
GFTFNTYAMN

SEQ ID NO: 153 - protein, 19 aa, synthetic construct
  source 1..19: note = CD3 Heavy Chain variable region CDR2
RIRSKYNNYATYYADSVKD

SEQ ID NO: 154 - protein, 14 aa, synthetic construct
  source 1..14: note = CD3 Heavy Chain variable region CDR3
HGNFGNSYVSWFAY

SEQ ID NO: 155 - protein, 14 aa, synthetic construct
  source 1..14: note = CD3 Light Chain variable region CDR1
RSSTGAVTTSNYAN

SEQ ID NO: 156 - protein, 7 aa, synthetic construct
  source 1..7: note = CD3 Light Chain variable region CDR2
GTNKRAP

SEQ ID NO: 157 - protein, 9 aa, synthetic construct
  source 1..9: note = CD3 Light Chain variable region CDR3
ALWYSNLWV

SEQ ID NO: 158 - protein, 125 aa, synthetic construct
  source 1..125: note = CD3 VH amino acid sequence
EVQLVESGGGLVQPGGSLKLSCAASGFEFNKYAMNWVRQAPGKGLEWVARIRSKYNNYETYYADSVKDRFTISRDDSKNTAYLQMNNLKTEDTAVYYCVRHGNFGNSLISYWAYWGQGTLVTVSS

SEQ ID NO: 159 - protein, 109 aa, synthetic construct
  source 1..109: note = CD3 VL amino acid sequence
QTVVTQEPSLTVSPGGTVTLTCGSSSGAVTSGNYPNWVQQKPGQAPRGLIGGTKFGAPGTPARFSGSLLGGKAALTLSGVQPEDEAEYYCVLWYSNRWVFGGGTKLTVL

SEQ ID NO: 160 - protein, 249 aa, synthetic construct
  source 1..249: note = CD3 scFv amino acid sequence
EVQLVESGGGLVQPGGSLKLSCAASGFEFNKYAMNWVRQAPGKGLEWVARIRSKYNNYETYYADSVKDRFTISRDDSKNTAYLQMNNLKTEDTAVYYCVRHGNFGNSLISYWAYWGQGTLVTVSSGGGGSGGGGSGGGGSQTVVTQEPSLTVSPGGTVTLTCGSSSGAVTSGNYPNWVQQKPGQAPRGLIGGTKFGAPGTPARFSGSLLGGKAALTLSGVQPEDEAEYYCVLWYSNRWVFGGGTKLTVL

SEQ ID NO: 161 - protein, 10 aa, synthetic construct
  source 1..10: note = CD3 Heavy Chain variable region CDR1
GFEFNKYAMN

SEQ ID NO: 162 - protein, 19 aa, synthetic construct
  source 1..19: note = CD3 Heavy Chain variable region CDR2
RIRSKYNNYETYYADSVKD

SEQ ID NO: 163 - protein, 14 aa, synthetic construct
  source 1..14: note = CD3 Heavy Chain variable region CDR3
HGNFGNSLISYWAY

SEQ ID NO: 164 - protein, 14 aa, synthetic construct
  source 1..14: note = CD3 Light Chain variable region CDR1
GSSSGAVTSGNYPN

SEQ ID NO: 165 - protein, 7 aa, synthetic construct
  source 1..7: note = CD3 Light Chain variable region CDR2
GTKFGAP

SEQ ID NO: 166 - protein, 9 aa, synthetic construct
  source 1..9: note = CD3 Light Chain variable region CDR3
VLWYSNRWV

SEQ ID NO: 167 - DNA, 1344 bp, synthetic construct
  source 1..1344: mol_type = other DNA; note = Hz45G10 Heavy chain nucleotide sequence
gaggtgcagctggttgaatctggcggaggactggttcagcctggcggatctctgagactgtcttgtgccgccagcggcttcaccttcagcgattatggcatgcactgggtccgacaggcccctggcaaaggacttgagtgggtcgcctacatcttcagcggcagcagcaccatctactacgccgacacagtgaagggcagattcaccatcagccgggacaacgccaagaacagcctgtacctgcagatgaactccctgagagccgaggacaccgccgtgtactattgtgccagagccgatggcagaggcgctatggattattggggccagggcaccctggtcaccgtttctagcgctagtaccaagggacccagcgtgttccctctggctcctagcagcaagtctacaagcggaggaacagccgctctgggctgcctggtcaaggattactttcccgagcctgtgaccgtgtcctggaatagcggagcactgacaagcggcgtgcacacctttccagctgtgctgcaaagcagcggcctgtactctctgagcagcgtggtcacagtgccaagctctagcctgggcacccagacctacatctgcaatgtgaaccacaagcctagcaacaccaaggtggacaagaaggtggaacccaagagctgcgacaagacccacacctgtcctccatgtcctgctccagaagctgctggcggcccttccgtgtttctgttccctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaaccaggtgtccctgtggtgcctcgtgaagggcttctacccttccgatatcgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggcaaa

SEQ ID NO: 168 - DNA, 642 bp, synthetic construct
  source 1..642: mol_type = other DNA; note = Hz45G10 Light chain nucleotide sequence
gacatccagatgacccagtctccatcctctctgtccgcctctgtgggcgacagagtgaccatcacctgtagagccagccaggatatctccaagttcctgaactggtatcagcagaagcccggcaaggcccctaagctgctgatctactacacctctcggctgcactctggcgtgccctctagattttctggctccggctctggcaccgactttacctttacaatctccagcctgcagcctgaggatatcgctacctacttctgccagcaaggcaacaccctgccttggacatttggcggaggcaccaagctggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 169 - protein, 674 aa, synthetic construct
  source 1..674: note = Heavy chain of anti-ILT3 binding region of F13
EVQLVESGGGLVQPGGSLRLSCAASGFTFSDYGMHWVRQAPGKGLEWVAYIFSGSSTIYYADTVKGRFTISRDNAKNSLYLQMNSLRAEDTAVYYCARADGRGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEVQLVESGGGLVQPGGSLRLSCAASGFTFSDYGMHWVRQAPGKGLEWVAYIFSGSSTIYYADTVKGRFTISRDNAKNSLYLQMNSLRAEDTAVYYCARADGRGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK